(19) United States
(12) Patent Application Publication
(10) Pub. No.: US 2009/0082295 A1
(43) Pub. Date: Mar. 26, 2009

(54) COMBINATIONS AND METHODS OF USING AN IMMUNOMODULATORY OLIGODEOXYNUCLEOTIDE

(75) Inventors: Jarl Ulf Birger Jungnelius, Mystic, CT (US); David Robert John Readett, Mystic, CT (US); Louis J. Denis, Pawcatcuk, CT (US)

Correspondence Address: SCULLY, SCOTT, MURPHY & PRESSER, P.C., 400 GARDEN CITY PLAZA, SUITE 300, GARDEN CITY, NY, 11530, US

(73) Assignee: PFIZER INC., New York, NY (US)

(21) Appl. No.: 12/093,265

(22) PCT Filed: Nov. 13, 2006
(86) PCT No.: PCT/US06/44003

§ 371 Date: Nov. 19, 2008

Related U.S. Application Data
(60) Provisional application No. 60/735,351, filed on Nov. 11, 2005.
(60) Provisional application No. 60/736,527, filed on Nov. 14, 2005.

(51) Int. Cl. A61K 31/7088; A61K 31/337; A61P 35/00

(52) U.S. Cl. 514/44; 514/449

(57) ABSTRACT

The present invention relates to combination therapies for the treatment of cancer. The combination of agents include oligonucleotides and one or more chemotherapeutic agents.

### FIELD OF THE INVENTION

This invention relates to combinations and methods of treating abnormal cell growth, such as cancer, in mammals, particularly in humans. In particular, the invention provides combination therapies and treatment regimens for treatment of cancers, using an immunostimulatory oligodeoxynucleotide.

### BACKGROUND

A traditional approach to treating cancer is to target the tumor itself with therapy. An alternative approach to cancer therapy is to target the immune system (“immunotherapy”) rather than and/or in addition to targeting the tumor itself. A potential benefit of immunotherapy is to provide improved efficacy by enhancing the patient's own immune response to tumors while minimizing deleterious effects to normal cells.

Bacterial DNA has immune stimulatory effects to activate B cells and natural killer cells (Tokunaga, T., et al., 1988. Jpn. J. Cancer Res. 79:682-686; Tokunaga, T., et al., 1984, JNCI 72:955-962; Messina, J. P., et al., 1991, J. Immunol. 147:1759-1764, and reviewed in Krieg, 1998, In: Applied Oligonucleotide Technology, C. A. Stein and A. M. Krieg, (Eds.), John Wiley and Sons, Inc., New York, N.Y., pp. 431-448). The immune stimulatory effects of bacterial DNA are a result of the presence of unmethylated CpG dinucleotides in particular base contexts (CpG motifs), which are common in bacterial DNA, but methylated and underrepresented in vertebrate DNA (Krieg et al, 1995 Nature 374:546-549; Krieg, 1999 Biochim. Biophys. Acta 93321:1-10). The immune stimulatory effects of bacterial DNA can be mimicked with synthetic oligodeoxynucleotides (ODN) containing these CpG motifs (referred to interchangeably hereinbelow as “CpG ODNs” or “immunostimulatory ODNs”). Such CpG ODN have highly stimulatory effects on human and murine leukocytes, inducing B cell proliferation, cytokine and immunoglobulin secretion, natural killer (NK) cell lytic activity, IFN-γ secretion, and activation of dendritic cells (DCs) and other antigen presenting cells to express costimulatory molecules and secrete cytokines, especially the Th1-like cytokines that are important in promoting the development of Th1-like T cell responses. The immune stimulatory effects of native phosphodiester backbone CpG ODN are highly CpG specific in that the effects are dramatically reduced if the CpG motif is methylated, changed to a GpC, or otherwise eliminated or altered (Krieg et al, 1995 Nature 374:546-549; Hartmann et al, 1999 Proc. Natl. Acad. Sci. USA 96:9305-10).

It was previously thought that the immune stimulatory effects required the CpG motif in the context of a purine-purine-CpG-pyrimidine-pyrimidine sequence (Krieg et al, 1995 Nature 374:546-549; Pisetsky, 1996 J. Immunol. 156:421-423; Hacker et al., 1998 EMBO J. 17:6230-6240; Lipford et al, 1998 Trends in Microbiol. 6:496-500). However, it is now clear that mouse lymphocytes respond quite well to phosphodiester CpG motifs not in this context (Yi et al., 1998 J. Immunol. 160:5898-5906) and the same is true of human B cells and dendritic cells (Hartmann et al, 1999 Proc, Natl. Acad. Sci. USA 96:9305-10; Liang, 1996 J. Clin. Invest. 98:1119-1129).

One class of GpG ODN is potent for activating B cells but is relatively weak in inducing IFN-alpha and NK cell activation; this class has been termed the B class. The B class CpG oligonucleotides typically are fully stabilized and include an unmethylated CpG dinucleotide within certain preferred base contexts. See, e.g., U.S. Pat. Nos. 6,194,388; 6,207,646; 6,214,806; 6,218,371; 6,239,116; and 6,339,068.

Although the individual use of CpG ODNs to induce an anti-tumor response hold great promise in the treatment of cancer, there remains a need to develop novel therapies to treat tumors, more particularly, solid tumors, with such immunotherapeutic approaches.

Worldwide more than 1.2 million cases of lung cancer are diagnosed each year. Mortality is very high and these diseases cause approximately 1.1 million deaths annually. In the United States, lung cancer is the leading cause of cancer mortality in both men and women. It is estimated that more than 172,570 new cases of lung cancer will be diagnosed in 2005 and more than 163,000 patients will die of lung cancer, accounting for nearly 30 percent of all cancer related deaths.

Approximately 80% of lung cancer is histologically defined as non-small cell and the remaining 20% as small cell. The majority of patients with non-small cell lung cancer (NSCLC) present with inoperable locally advanced (Stage IIIB) or metastatic (Stage IV) disease for which no curative therapy is available. For these patients, platinum-based chemotherapy has been shown to provide a modest increase in median survival when compared to best supportive care alone in meta-analyses of individual studies. Subsequently, a number of platinum-based doublet (i.e., two chemotherapeutic agents used in combination) combination regimens have become the clinical and regulatory standard of care based on trials in which they demonstrated improved survival when compared to platinum alone or to other, non-platinum, single agent treatments. Worldwide, commonly prescribed doublet chemotherapy regimens would consist of either cisplatin or carboplatin combined with paclitaxel, docetaxel, gemcitabine or vinorelbine. Geographic patterns of preference exist; paclitaxel plus carboplatin is the most commonly prescribed regimen in the United States whilst gemcitabine or vinorelbine combined with either cisplatin or carboplatin is prescribed most commonly in Europe. Several large randomized Phase 3 trials have compared different platinum-based regimens but no regimen has demonstrated clear superiority in terms of response rate, progression free survival or overall survival. Median survival for the different treatment arms in these trials ranged from 7.4 to 9.9 months. There are differences in the toxicity profiles for the various doublet combinations, but no single regimen is considered markedly less toxic than the others. Consequently, all platinum-based doublet regimens could be considered appropriate standard of care for first line treatment of advanced NSCLC and the choice of regimen is based largely on individual physician and patient preference.

A number of randomized clinical trials have investigated the addition of a third cytotoxic agent to these standard platinum-based combinations. Although some triplet (i.e., three chemotherapeutic agents used in combination) combinations have been able to improve objective response rate, none has been able to improve overall survival. More recently novel targeted agents such as the epidermal growth factor receptor (EGFR) tyrosine kinase inhibitors gefitinib (Gefitinib®) and erlotinib (Tarceva®) and the vascular endothelial growth factor (VEGF) targeting monoclonal antibody bevacizumab (Avastin®) have also been tested in patients with NSCLC. Gefitinib and erlotinib both demonstrated clinical activity in refractory NSCLC but neither was able to improve clinical outcome when added to standard 1^{st} line platinum-based chemotherapy. A randomized Phase 3 trial investigating the addition of bevacizumab to 1^{st} line paclitaxel plus carboplatin chemotherapy, has recently been completed. Due to observation of bevacizumab-related pulmonary hemorrhage in the preceding Phase 2 trial, patients with squamous cell histology, central tumors, gross hemoptysis or patients taking coumadin, aspirin or antiplatelet therapy were excluded from this Phase 3 trial. Recent interim results from the trial reported a 23% improvement in the median overall survival for the paclitaxel/carboplatin plus bevacizumab treatment arm (12.5 months vs. 10.2 months−p=0.007) Of note, an exploratory subgroup analysis did not demonstrate a survival benefit for female patients despite a treatment effect for response rate and progression free survival. Despite the eligibility restrictions, CTC Grade 31415 bleeding occurred in 4.5% of patients in the paclitaxel/carboplatin plus bevacizumab arm, including 5 deaths related to hemoptysis, compared to only 0.7% of patients in the paclitaxel/carboplatin alone arm. It remains to be determined how these data will impact the use of bevacizumab in 1^{st} line NSCLC.

There is still a clear need for new, novel treatment options for patients with newly diagnosed advanced NSCLC. Recent data have suggested that vaccine-based immunotherapy may have clinical utility in the treatment of advanced NSCLC. Other immunotherapeutic approaches remain attractive options, as they could potentially provide safe and effective treatment modalities for advanced NSCLC. Moreover, to a large extent, the unmet medical need for treatment of NSCLC is illustrative of other types of cancer, including but not limited to SCLC, breast cancer, melanoma, cutaneous T-cell lymphoma, and other forms of non-Hodgkin's lymphoma. Therefore, there is also a clear need for novel treatment options for patients with various forms of cancer.

### SUMMARY

Development of new therapeutic regimens, particularly those capable of augmenting or potentiating the anti-tumor activity of the immune system of the patient, while reducing the cytotoxic side effects of current chemotherapeutics, is necessary. The present invention provides such regimens.

Therefore, the invention provides a method of treating or preventing cancer in a patient in need of such treatment, wherein the method comprises (a) a therapeutic regimen comprising administering to the patient simultaneously, semi-simultaneously, separately or sequentially a therapeutically effective amount of a CpG ODN in combination with a therapeutically effective amount of a chemotherapeutic agent selected from the group consisting of 5-(5-fluoro-2-oxo-1,2-dihydroindol-(3Z)-ylidenemethyl)-2,4-dimethyl-1H-pyrrole-3-carboxylic acid (2-diethylaminoethyl)-amide, epirubicin, doxorubicin, paclitaxel, docetaxel, gemcitabine, vinorelbine, irinotecan, pemetrexed, mitomycin, vincristine, capecitabine, cyclophosphamide, methotrexate, leucovorin, trastuzumab, lapatinib, vinblastine, vindesine, cisplatin, carboplatin, oxaliplatin, gefitinib, erlotinib, TLK-286, cetuximab, bevacizumab, etoposide, bleomycin, 5-FU, melphalan, ZD 6474, ZD 2171, UFT, S1, ifosfamide, thiotepa, temozolomide, talabostat, interferon, tamoxifen, raloxifene, exemestane, anastrozole, zoladex, letrozole, megace, abraxane, bisphosphonate, temozolomide, fragmin, faslodex, irinotecan, oxaliplatin, DTIC, interferon, interleukin, sorafenib, IL-2, and combinations thereof;

and optionally (b) administering to the patient a maintenance regimen comprising a maintenance dose of a CpG ODN.

Preferably, the method of the present invention provides a therapeutically effective amount of a CpG ODN comprising a therapeutic dose of about 0.01 to 5.0 mg/kg, preferably, about 0.01 to 2.5 mg/kg, most preferably about 0.05 to 1.0 mg/kg, and still more preferably about 0.2 mg/kg.

The invention provides a method of treatment or prevention wherein the therapeutic dose is (a) administered before administration of the chemotherapeutic agent, (b) after administration of the chemotherapeutic agent, (c) administered to the patient about 1 week to 3 weeks before the administration of the chemotherapeutic agent, (d) administered to the patient about 1 week before the administration of the chemotherapeutic agent, (e) administered to the patient about 1 week to 3 weeks after administration of the chemotherapeutic agent, (f) administered to the patient about 1 week after administration of the chemotherapeutic agent, and/or (g) wherein the method further comprises a treatment regimen comprising a therapy selected from the group consisting of surgery, radiation therapy, or a combination thereof.

The present invention also contemplates the administration of a maintenance dose of a CpG ODN of about 0.01 to 5.0 mg/kg, preferably, about 0.01 to 2.5 mg/kg, more preferably about 0.05 to 1.0 mg/kg, and most preferably about 0.2 mg/kg.

The invention also provides a method of treating or preventing non-small cell lung cancer (NSCLC) in a patient in need of such treatment, wherein the method comprises (a) a therapeutic regimen comprising administering to the patient simultaneously, semi-simultaneously, separately or sequentially a therapeutically effective amount of a CpG ODN in combination with a therapeutically effective amount of a chemotherapeutic agent selected from the group consisting of 5-(5-fluoro-2-oxo-1,2-dihydroindol-(3Z)-ylidenemethyl)-2,4-dimethyl-1H-pyrrole-3-carboxylic acid (2-diethylaminoethyl)-amide, paclitaxel, docetaxel, gemcitabine, vinorelbine, irinotecan, pemetrexed, mitomycin, vincristine, vinblastine, vindesine, cisplatin, carboplatin, oxaliplatin, gefitinib, erlotinib, TLK-286, cetuximab, bevacizumab, etoposide, bleomycin, 5-FU, melphalan, ZD 6474, ZD 2171, UFT, S1, ifosfamide, thiotepa, temozolomide, talabostat, interferon, and combinations thereof; and optionally (b) administering to the patient a maintenance regimen comprising a maintenance dose of a CpG ODN.

In a preferred embodiment, the invention provides a method of treating NSCLC as described hereinabove, comprising simultaneous, semi-simultaneous, separate or sequential administration of a therapeutically effective amount of a CpG ODN and a therapeutically effective amount of (a) a first chemotherapeutic agent selected from the group consisting of 5-(5-fluoro-2-oxo-1,2-dihydroindol-(3Z)-ylidenemethyl)-2,4-dimethyl-1H-pyrrole-3-carboxylic acid (2-diethylaminoethyl)-amide, paclitaxel, docetaxel, gemcitabine, vinorelbine, irinotecan, pemetrexed, mitomycin, vincristine, vinblastine, vindesine, cisplatin, carboplatin, oxaliplatin, gefitinib, erlotinib, TLK-286, cetuximab, bevacizumab, etoposide, bleomycin, 5-FU, melphalan, ZD 6474, ZD 2171, UFT, S1, ifosfamide, thiotepa, temozolomide, talabostat, interferon; and (b) a second chemotherapeutic agent selected from the group consisting of 5-(5-fluoro-2-oxo-1,2-dihydroindol-(3Z)-ylidenemethyl)-2,4-dimethyl-1H-pyrrole-3-carboxylic acid (2-diethylaminoethyl)-amide, paclitaxel, docetaxel, gemcitabine, vinorelbine, irinotecan, pemetrexed, mitomycin, vincristine, vinblastine, vindesine, cisplatin, carboplatin, oxaliplatin, gefitinib, erlotinib, TLK-286, cetuximab, bevacizumab, etoposide, bleomycin, 5-FU, melphalan, ZD 6474, ZD 2171, UFT, S1, ifosfamide, thiotepa, temozolomide, talabostat, interferon; wherein said first and second chemotherapeutic agents are different; and optionally (c) administering to the patient a maintenance regimen comprising a maintenance dose of a CpG ODN; with the proviso that if the first chemotherapeutic agent is selected from cisplatin or carboplatin then the second chemotherapeutic agent is not paclitaxel or docetaxel.

In a preferred embodiment, the CpG ODN is selected from the group consisting of PF3512676, 1018 ISS, Genasense, and IMOxine®, and preferably, the CpG ODN is PF3512676.

In one embodiment of the invention, the method contemplates the administration of a chemotherapeutic agent is selected from the group consisting of 5-(5-fluoro-2-oxo-1,2-dihydroindol-(3Z)-ylidenemethyl-2,4-dimethyl-1H-pyrrole-3-carboxylic acid (2-diethylaminoethyl)-amide, pemetrexed, gemcitabine, bevacizumab, carboplatin, erlotinib, and combinations thereof, and said therapeutic regimen further comprises administering cisplatin.

Further, the invention provides a method of treating or preventing NSCLC wherein the therapeutically effective amount of a CpG ODN is a therapeutic dose of about 0.01 to 5.0 mg/kg, preferably about 0.01 to 2.5 mg/kg, more preferably about 0.05 to 1.0 mg/kg, and most preferably about 0.2 mg/kg.

Moreover, the invention contemplates a method of treating or preventing NSCLC wherein the therapeutic dose is (a) administered before administration of the chemotherapeutic agent, (b) after administration of the chemotherapeutic agent, (c) administered to the patient about 1 week to 3 weeks before the administration of the chemotherapeutic agent, (d) administered to the patient about 1 week before the administration of the chemotherapeutic agent, (e) administered to the patient about 1 week to 3 weeks after administration of the chemotherapeutic agent, (f) administered to the patient about 1 week after administration of the chemotherapeutic agent, and/or (g) wherein the method further comprises a treatment regimen comprising a therapy selected from the group consisting of surgery, radiation therapy, or a combination thereof.

The present invention also contemplates the administration of a maintenance dose of a CpG ODN of about 0.01 to 5.0 mg/kg, preferably, about 0.01 to 2.5 mg/kg, more preferably about 0.05 to 1.0 mg/kg, and most preferably about 0.2 mg/kg.

The invention also provides a method of treating or preventing breast cancer in a patient in need of such treatment, wherein the method comprises (a) a therapeutic regimen comprising administering to the patient simultaneously, semi-simultaneously, separately or sequentially a therapeutically effective amount of a GpG ODN in combination with a therapeutically effective amount of a chemotherapeutic agent selected from the group consisting of 5-(5-fluoro-2-oxo-1,2-dihydroindol-(3Z)-ylidenemethyl)-2,4-dimethyl-1H-pyrrole-3-carboxylic acid (2-diethylaminoethyl)-amide, epirubicin, doxorubicin, paclitaxel, docetaxel, capecitabine, vincristine, gemcitabine, 5-FU, cyclophosphamide, methotrexate, leucovorin, trastuzumab, bevacizumab, lapatinib, erlotinib, gefitinib, pemetrexed, tamoxifen, raloxifene, exemestane, anastrozole, zoladex, letrozole, megace, abraxane, bisphosphonate, temozolomide, fragmin, faslodex, irinotecan, cisplatin, carboplatin, oxaliplatin, and combinations thereof; and optionally (b) administering to the patient a maintenance regimen comprising a maintenance dose of a CpG ODN. In a preferred embodiment, the CpG ODN is selected from the group consisting of PF3512676, 1018 ISS, Genasense, and IMOxine®, and preferably, the CpG ODN is PF3512676.

Further, the invention provides a method of treating or preventing breast cancer wherein the therapeutically effective amount of a CpG ODN is a therapeutic dose of about 0.01 to 5.0 mg/kg, preferably about 0.01 to 2.5 mg/kg, more preferably about 0.05 to 1.0 mg/kg, and most preferably about 0.2 mg/kg.

Moreover, the invention contemplates a method of treating or preventing breast cancer wherein the therapeutic dose is (a) administered before administration of the chemotherapeutic agent, (b) after administration of the chemotherapeutic agent, (c) administered to the patient about 1 week to 3 weeks before the administration of the chemotherapeutic agent, (d) administered to the patient about 1 week before the administration of the chemotherapeutic agent, (e) administered to the patient about 1 week to 3 weeks after administration of the chemotherapeutic agent, (f) administered to the patient about 1 week after administration of the chemotherapeutic agent, and/or (g) wherein the method further comprises a treatment regimen comprising a therapy selected from the group consisting of surgery, radiation therapy, or a combination thereof.

The present invention also contemplates the administration of a maintenance dose of a CpG ODN of about 0.01 to 5.0 mg/kg, preferably, about 0.01 to 2.5 mg/kg, more preferably about 0.05 to 1.0 mg/kg, and most preferably about 0.2 mg/kg.

The invention further provides a method of treating or preventing melanoma in a patient in need of such treatment, wherein the method comprises (a) a therapeutic regimen comprising administering to the patient simultaneously, semi-simultaneously, separately or sequentially a therapeutically effective amount of a CpG ODN in combination with a therapeutically effective amount of a chemotherapeutic agent selected from the group consisting of DTIC, binblastine, bincristine, vindesine, temozolomide, interferon, interleukin, and combinations thereof; and optionally (b) administering to the patient a maintenance regimen comprising a maintenance dose of a CpG ODN. In a preferred embodiment, the CpG ODN is selected from the group consisting of PF3512676, 1018 ISS, Genasense, and IMOxine®, and preferably, the CpG ODN is PF3512676.

Further, the invention provides a method of treating melanoma wherein the therapeutically effective amount of a CpG ODN is a therapeutic dose of about 0.01 to 5.0 mg/kg, preferably about 0.01 to 2.5 mg/kg, more preferably about 0.05 to 1.0 mg/kg, and most preferably about 0.2 mg/kg.

Moreover, the invention contemplates a method of treating melanoma wherein the therapeutic dose is (a) administered before administration of the chemotherapeutic agent, (b) after administration of the chemotherapeutic agent, (c) administered to the patient about 1 week to 3 weeks before the administration of the chemotherapeutic agent, (d) administered to the patient about 1 week before the administration of the chemotherapeutic agent, (e) administered to the patient about 1 week to 3 weeks after administration of the chemotherapeutic agent, (f) administered to the patient about 1 week after administration of the chemotherapeutic agent, and/or (g) wherein the method further comprises a treatment regimen comprising a therapy selected from the group consisting of surgery, radiation therapy, or a combination thereof.

The present invention also contemplates the administration of a maintenance dose of a CpG ODN of about 0.01 to 5.0 mg/kg, preferably, about 0.01 to 2.5 mg/kg, more preferably about 0.05 to 1.0 mg/kg, and most preferably about 0.2 mg/kg.

The present invention further provides a method of treating or preventing renal cell carcinoma in a patient in need of such treatment, wherein the method comprises (a) a therapeutic regimen comprising administering to the patient simultaneously, semi-simultaneously, separately or sequentially a therapeutically effective amount of a CpG OGN in combination with a therapeutically effective amount of a chemotherapeutic agent selected from the group consisting of 5-(5-fluoro-2-oxo-1,2-dihydroindol-(3Z)-ylidenemethyl)-2,4-dimethyl-1H-pyrrole-3-carboxylic acid (2-diethylaminoethyl)-amide, sorafenib, IL-2, imatinib, bevacizumab, gemcitabine, cisplatin, carboplatin, paclitaxel, docetaxel, and combinations thereof; and optionally (b) administering to the patient a maintenance regimen comprising a maintenance dose of a CpG ODN. In a preferred embodiment, the CpG OGN is selected from the group consisting of PF3512676, 1018 ISS, Genasense, and IMOxine®, and preferably, the CpG ODN is PF3512576.

Further, the invention provides a method of treating or preventing RCC wherein the therapeutically effective amount of a CpG ODN is a therapeutic dose of about 0.01 to 5.0 mg/kg, preferably about 0.01 to 2.5 mg/kg, more preferably about 0.05 to 1.0 mg/kg, and most preferably about 0.2 mg/kg.

Moreover, the invention contemplates a method of treating or preventing RCC wherein the therapeutic dose is (a) administered before administration of the chemotherapeutic agent, (b) after administration of the chemotherapeutic agent, (c) administered to the patient about 1 week to 3 weeks before the administration of the chemotherapeutic agent, (d) administered to the patient about 1 week before the administration of the chemotherapeutic agent, (e) administered to the patient about 1 week to 3 weeks after administration of the chemotherapeutic agent, (f) administered to the patient about 1 week after administration of the chemotherapeutic agent, and/or (g) wherein the method further comprises a treatment regimen comprising a therapy selected from the group consisting of surgery, radiation therapy, or a combination thereof.

The present invention also contemplates the administration of a maintenance dose of a CpG ODN of about 0.01 to 5.0 mg/kg, preferably, about 0.01 to 2.6 mg/kg, more preferably about 0.05 to 1.0 mg/kg, and most preferably about 0.2 mg/kg.

These and other embodiments of the invention is described in greater detail herein.

Each of the limitations of the invention can encompass various embodiments of the invention. It is therefore anticipated that each of the limitations of the invention involving any one element or combinations of elements can be included in each aspect of the invention. This invention is not limited in its application to the details of construction and the arrangement of components set forth in the following description or illustrated in the drawings. The invention is capable of other embodiments and of being practiced or of being carried out in various ways.

The phraseology and terminology used herein is for the purpose of description and should not be regarded as limiting. The use of “including”, “comprising”, or “having”, “containing”, “involving”, and variations thereof herein, is meant to encompass the items listed thereafter and equivalents thereof as well as additional items.

### BRIEF DESCRIPTION OF THE FIGURE

FIG. 1 depicts the structure of PF3512676, a preferred CpG ODN used in the present invention.

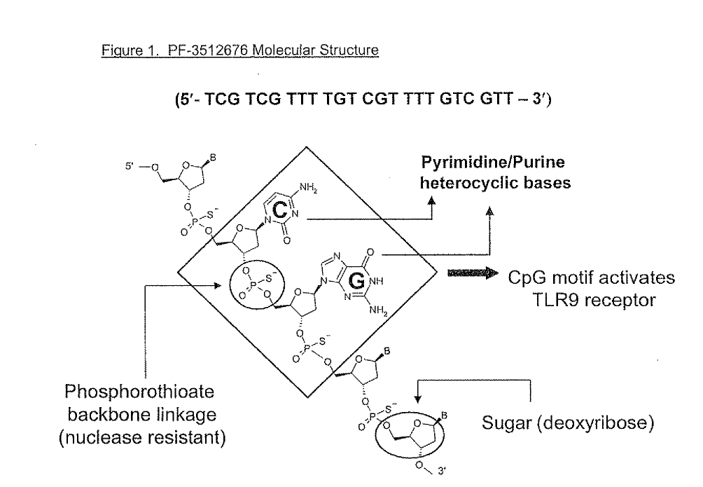

### DETAILED DESCRIPTION

### I. Definitions

Unless otherwise defined herein, scientific and technical terms used in connection with the present invention shall have the meanings that are commonly understood by those of ordinary skill in the art. Further, unless otherwise required by context, singular terms shall include pluralities and plural terms shall include the singular. Generally, nomenclatures used in connection with, and techniques of, cell and tissue culture, molecular biology, immunology, microbiology, genetics and protein and nucleic acid chemistry and hybridization described herein are those well known and commonly used in the art.

The methods and techniques of the present invention are generally performed according to methods well known in the art and as described in various general and more specific references that are cited and discussed throughout the present specification unless otherwise indicated. Such references include, e.g., Sambrook and Russell, Molecular Cloning, A Laboratory Approach, Cold Spring Harbor Press, Cold Spring Harbor, N.Y. (2001), Ausubel et al., Current Protocols in Molecular Biology, John Wiley & Sons, NY (2002), and Harlow and Lane Antibodies: A Laboratory Manual, Cold Spring Harbor Laboratory Press, Cold Spring Harbor, N.Y. (1990), which are incorporated herein by reference. Enzymatic reactions and purification techniques are performed according to manufacturer's specifications, as commonly accomplished in the art or as described herein. The nomenclatures used in connection with, and the laboratory procedures and techniques of, analytical chemistry, synthetic organic chemistry, and medicinal and pharmaceutical chemistry described herein are those well known and commonly used in the art. Standard techniques are used for chemical syntheses, chemical analyses, pharmaceutical preparation, formulation, and delivery, and treatment of patients.

As used herein, each of the following terms has the meaning associated with it in this section. The articles “a” and “an” are used herein to refer to one or to more than one (i.e., to at least one) of the grammatical object of the article. By way of example, “an element” means one element or more than one element.

A “pharmaceutical composition” refers to a mixture of one or more of the compounds described herein, or physiologically/pharmaceutically acceptable salts, solvates, hydrates or prodrugs thereof, with other components, such as physiologically/pharmaceutically acceptable carriers and excipients. The purpose of a pharmaceutical composition is to facilitate administration of a compound to an organism.

As used herein, a “physiologically/pharmaceutically acceptable carrier” refers to a carrier or diluent that does not cause significant irritation to an organism and does not abrogate the biological activity and properties of the administered compound.

A “pharmaceutically acceptable excipient” refers to an inert substance added to a pharmaceutical composition to further facilitate administration of a compound. Examples, without limitation, of excipients include calcium carbonate, calcium phosphate, various sugars and types of starch, cellulose derivatives, gelatin, vegetable oils and polyethylene glycols.

As used herein, the term “pharmaceutically acceptable salt” refers to those salts which retain the biological effectiveness and properties of the parent compound. Such salts include acid addition salts, which can be obtained by reaction of the free base of the parent compound with inorganic acids such as hydrochloric acid, hydrobromic acid, nitric acid, phosphoric acid, sulfuric acid, and perchloric acid and the like, or with organic acids such as acetic acid, oxalic acid, (D) or (L) malic acid, maleic acid, methanesulfonic acid, ethanesulfonic acid, p-toluenesulfonic acid, salicylic acid, tartaric acid, citric acid, succinic acid or malonic acid and the like.

By the term “effective amount”, or “therapeutically effective amount,” as used herein, is meant an amount that when administered to a mammal, preferably a human, mediates a detectable therapeutic response compared to the response detected in the absence of the compound. A therapeutic response, such as, but not limited to, increased overall survival, inhibition of and/or decreased tumor growth (including tumor size stasis), tumor size, metastasis, and the like, can be readily assessed by a plethora of art-recognized methods, including, e.g., such methods as disclosed herein.

The skilled artisan would understand that the effective amount of the CpG ODN, compound or composition administered herein varies and can be readily determined based on a number of factors such as the disease or condition being treated, the stage of the disease, the age and health and physical condition of the mammal being treated, the severity of the disease, the particular CpG ODN compound being administered, and the like.

A “therapeutic effective amount”, or “effective amount,” is intended to qualify the amount of an agent required to detectably reduce to some extent one or more of the symptoms of a neoplasia disorder, including, but is not limited to: (1) reduction in the number of cancer cells; (2) reduction in tumor size; (3) inhibition (i.e., slowing to some extent, preferably stopping) of cancer cell infiltration into peripheral organs; (4) inhibition (i.e., slowing to some extent, preferably stopping) of tumor metastasis; (5) inhibition, to some extent, of tumor growth; (6) relieving or reducing to some extent one or more of the symptoms associated with the disorder; (7) relieving or reducing the side effects associated with the administration of anticancer agents; and/or (8) increasing, to some extent, the overall survival of a patient relative to that observed for the standard of care for a given tumor type or neoplastic disorder.

A “maintenance effective amount” is intended to qualify the amount of an agent required to detectably maintain the therapeutic benefit achieved during a therapeutic regimen, including, but not limited to (1) inhibiting an increase in the number of cancer cells; (2) inhibiting an increase in tumor size; (3) inhibiting cancer cell infiltration into peripheral organs; (4) inhibiting tumor metastases; (5) relieving or reducing to some extent one or more of the symptoms associated with the disorder; and/or (6) inhibiting a recurrence or onset of one or more of the symptoms associated with the disorder.

Combined with the teachings provided herein, by choosing among the various active compounds and weighing factors such as potency, relative bioavailability, patient body weight, severity of adverse side-effects and preferred mode of administration, an effective prophylactic or therapeutic treatment regimen or maintenance phase can be planned which does not cause substantial toxicity and yet is entirely effective to treat the particular subject.

The therapeutic or maintenance effective amount for any particular application can vary depending on such factors as the disease or condition being treated, the severity of the disease or condition, and the health and size of the subject. One of ordinary skill in the art can empirically determine the therapeutic effective amount of an immunostimulatory ODN, e.g., CpG ODN, 1018 ISS or IMOxine®, chemotherapeutic agents, and/or other therapeutic agent without undue experimentation.

As used herein, to “treat” means reducing the frequency with which symptoms of a disease (i.e., tumor growth and/or metastasis, or other effect mediated by the numbers and/or activity of immune cells, and the like) are experienced by a patient. The term includes the administration of the CpG ODN, compounds or agents of the present invention to prevent or delay the onset of the symptoms, complications, or biochemical indicia of a disease (e.g., elevation of PSA level in prostate cancer), alleviating the symptoms or arresting or inhibiting further development of the disease, condition, or disorder. Treatment may be prophylactic (to prevent or delay the onset of the disease, or to prevent the manifestation of clinical or subclinical symptoms thereof) or therapeutic suppression or alleviation of symptoms after the manifestation of the disease.

As used herein, to “prevent” means inhibiting the onset or development of symptoms of a disease (i.e., tumor growth and/or metastasis, or other effect mediated by the numbers and/or activity of immune cells, and the like) experienced by a patient. The term includes the administration of the CpG ODN, compounds or agents of the present invention to inhibit or delay the onset of the symptoms, complications, or biochemical indicia of a disease (e.g., elevation of PSA level in prostate cancer).

The therapeutically effective amount or maintenance effective amount of immunostimulatory ODN and/or chemotherapeutic agent alone or together can be initially determined from animal models. A therapeutically or maintenance effective dose can also be determined from human data for the specific immunostimulatory ODN and/or chemotherapeutic agents or for other compounds which are known to exhibit similar pharmacological activities. Higher doses may be required for parenteral administration. The applied dose can be adjusted based on the relative bioavailability and potency of the administered compound. Adjusting the dose to achieve maximal efficacy based on the methods described above and other methods as are well-known in the art is well within the capabilities of the ordinarily skilled artisan.

“Instructional material,” as that term is used herein, includes a publication, a recording, a diagram, or any other medium of expression which can be used to communicate the usefulness of the compound, combination, and/or composition of the invention in the kit for affecting, alleviating or treating the various diseases or disorders recited herein. Optionally, or alternately, the instructional material can describe one or more methods of alleviating the diseases or disorders in a cell, a tissue, or a mammal, including as disclosed elsewhere herein.

The instructional material of the kit may, for example, be affixed to a container that contains the CpG ODN, compound and/or composition of the invention or be shipped together with a container that contains the compound and/or composition. Alternatively, the instructional material may be shipped separately from the container with the intention that the recipient uses the instructional material and the compound cooperatively.

The CpG ODN and/or chemotherapeutic agent of the invention may be provided in a medicinal dispenser. A medical dispenser is a package defining a plurality of medicinal storage compartments, each compartment for housing an individual unit of medicament. An entire medicinal course of treatment is housed in a plurality of medicinal storage compartments.

A package defining a plurality of medicinal storage compartments may be any type of disposable pharmaceutical package or card that holds medicaments in individual compartments. For example, the package is a blister package constructed from a card, which may be made from stiff paper material, a blister sheet and backing sheet. Such cards are well known to those of ordinary skill in the art.

As an example, a medicinal dispenser may house an entire medicinal course of treatment. The dispenser may include the day indicia to indicate which day the individual units of medicament are to be taken. These may be marked along a first side of the medicinal package. The dose indicia may also be marked, for example along a second side of the medicinal package perpendicular to the first side of the medicinal package, thereby indicating the time which the individual unit of medicament should be taken. The unit doses may be contained in the dispenser which is a blister pack.

Except when noted, the terms “patient” or “subject” are used interchangeably and refer to mammals such as human patients and non-human primates, as well as veterinary subjects such as rabbits, rats, and mice, and other animals. Preferably, patient refers to a human.

“Combination therapy” embraces the administration of an immunostimulatory ODN, e.g., CpG ODN, PF3512676, 1018 ISS, IMOxine®, and a chemotherapeutic agent as part of a specific treatment regimen optionally including a maintenance phase, intended to provide a beneficial effect from the co-action of these therapeutic agents. The beneficial effect of the combination includes, but is not limited to, pharmacokinetic or pharmacodynamic co-action resulting from the combination of therapeutic agents. Administration of these therapeutic agents in combination typically is carried out over a defined time period (usually minutes, hours, days or weeks depending upon the combination selected). “Combination therapy” generally is not intended to encompass the administration of two or more of these therapeutic agents as part of separate monotherapy regimens that incidentally and arbitrarily result in the combinations of the present invention.

“Combination therapy” embraces administration of these therapeutic agents in a sequential manner, that is, wherein each therapeutic agent is administered at a different time, as well as administration of these therapeutic agents, or at least two of the therapeutic agents, in a substantially simultaneous manner. Substantially simultaneous administration can be accomplished, for example, by administering to the subject a single capsule having a fixed ratio of each therapeutic agent or in multiple, single capsules for each of the therapeutic agents. Sequential or substantially simultaneous administration of each therapeutic agent can be effected by any appropriate route including, but not limited to, oral routes, intravenous routes, intramuscular, subcutaneous routes, and direct absorption through mucous membrane tissues. The therapeutic agents can be administered by the same route or by different routes. For example, a first therapeutic agent (e.g., CpG ODN) can be administered by subcutaneous injection, and a second agent (e.g., a chemotherapeutic agent) can be administered intravenously. Further, a first therapeutic agent of the combination selected may be administered by intravenous injection while the other therapeutic agents of the combination may be administered orally. Alternatively, for example, both the therapeutic agents may be administered orally or both therapeutic agents may be administered by intravenous or subcutaneous injection.

In the present specification the term “sequential” means, unless otherwise specified, characterized by a regular sequence or order, e.g., if a dosage regimen includes the administration of a CpG ODN and a chemotherapeutic agent, a sequential dosage regimen could include administration of the CpG ODN before, simultaneously, substantially simultaneously, or after administration of the chemotherapeutic agent, but both agents will be administered in a regular sequence or order. The term “separate” means, unless otherwise specified, to keep apart one from the other. The term “simultaneously” means, unless otherwise specified, happening or done at the same time, i.e., the compounds of the invention are administered at the same time. The term “substantially simultaneously” means that the compounds are administered within minutes of each other (e.g., within 10 minutes of each other) and intends to embrace joint administration as well as consecutive administration, but if the administration is consecutive it is separated in time for only a short period (e.g., the time it would take a medical practitioner to administer two compounds separately). As used herein, concurrent administration and substantially simultaneous administration are used interchangeably. Sequential administration refers to temporally separated administration of the ODN and the chemotherapeutic agent.

“Combination therapy” also can embrace the administration of the therapeutic agents as described above in further combination with other biologically active ingredients (such as, but not limited to, a second and different antineoplastic agent, a dendritic vaccine or other tumor vaccine) and non-drug therapies (such as, but not limited to, surgery or radiation treatment or both). Where the combination therapy further comprises radiation treatment, the radiation treatment may be conducted at any suitable time so long as a beneficial effect from the co-action of the combination of the therapeutic agents and radiation treatment is achieved. For example, in appropriate cases, the beneficial effect is still achieved when the radiation treatment is temporally removed from the administration of the therapeutic agents, perhaps by days or even weeks.

As used herein, the term “adjuvant therapy” refers to treatment given after the primary treatment, including, without limitation, radiation, chemotherapy, hormone therapy, etc. The goal of adjuvant therapy is to increase the patients' chances of remission or cure, to increase the patients' overall survival benefit, and to help decrease the risk of recurrence. Therefore, it will be understood that if the CpG ODN is administered as an adjuvant, it will be administered to the patient after the primary treatment, e.g., the patient is given a regimen of radiation and/or chemotherapy, followed by a course of CpG ODN. In this regard, the dose of CpG ODN may be considered a therapeutic dose or a maintenance dose, depending on the goals of the adjuvant therapy. The term “neoadjuvant therapy” refers to treatment given before the primary treatment, including, without limitation, radiation, chemotherapy, etc. In the neoadjuvant setting, the dose of CpG ODN is a therapeutic dose.

The term “first-line therapy” refers to the first type of therapy given for a condition or disease, or the first therapy of choice for the treatment of a particular type of cancer. It necessarily follows that the term “second-line therapy” therefore refers to the treatment given when the initial or first-line therapy is unsuccessful, and “third-line therapy” refers to a treatment or treatment regimen that is given when both the initial treatment and the subsequent treatment are unsuccessful.

“Metastasis” or “metastatic cancer” refers to the spread of cancer from one part of the body to another. A tumor formed by cells that have spread is called a “metastatic tumor” or “metastasis”. The metastatic tumor contains cells that are like those in the original (primary) tumor.

“Remission” refers to a decrease in or disappearance of signs and symptoms of cancer. In partial remission, some, but not all, signs and symptoms of cancer are undetectable. In complete remission, all signs and symptoms of cancer have disappeared, although cancer cells may remain in the body.

“Overall survival” refers to the percentage of patients that have survived for a defined period of time. The term is usually reported as time since diagnosis or treatment, and is synonymous with “survival rate”.

### I. Methods of Treatment

The present invention contemplates methods of treating various tumor types. In a preferred embodiment, the invention particularly relates to methods of treating lung cancer, breast cancer, melanoma, cutaneous T-cell lymphoma, and non-Hodgkin's lymphoma. The following is a brief summary of the current treatment options for patients diagnosed with one of these cancers.

(a) Lung Cancer

As discussed above, it will be understood to one of ordinary skill in the art that lung cancer is divided into two major types, non-small cell lung cancer (NSCLC) and small cell lung cancer (SCLC). Each type of lung cancer grows and spreads in different ways and may be treated differently. There are 3 subtypes of NSCLC: squamous cell carcinoma, adenocarcinoma (including bronchioloalveolar carcinoma), and large cell undifferentiated carcinoma. The subtype of NSCLC, however, does not influence treatment options. SCLC is often referred to alternatively as oat cell cancer, small cell undifferentiated carcinoma, and poorly differentiated neuroendocrine carcinoma.

NSCLC is often treated by combining one or more chemotherapeutic agents and chemotherapy is often administered in cycles, with each period of treatment followed by a recovery period Chemotherapy cycles generally last about 21 to 28 days, and initial treatment typically involves 4-6 cycles. The recovery period often varies according to the type of therapy given in a cycle as well as the condition of the patient. The drug combinations most frequently used for first line chemotherapy for NSCLC are cisplatin or carboplatin combined with one of the following agents: paclitaxel, docetaxel, gemcitabine, vinorelbine, ilrinotecan, etoposide, or vinblastine. Chemotherapy or targeted therapy used for second line treatment for NSCLC include docetaxel alone or gefitinib.

The drug combinations most frequently used for initial chemotherapy for SCLC are cisplatin and etoposide or carboplatin and etoposide (for limited stage), and cisplatin and etoposide, carboplatin and etoposide, or cisplatin or irinotecan (for extensive stage). Chemotherapy drugs used if there has been a relapse of the SCLC include: (a) ifosfamide, paclitaxel, docetaxel, or gemcitabine, if the relaps occurred within 2 to 3 months; (b) topotecan, irinotecan, cyclophosphamide/doxorubicin/vincristine, gem citabine, paclitaxel, docetaxel, oral etoposide, methotrexate, or vinorelbine, if the relapse occurred from 2 to 3 months to 6 months; (c) for relapses after 6 months, the original chemotherapy can be repeated.

(b) Breast Cancer

The most commonly used combinations for the treatment of breast cancer are: (a) Cyclophosphamide (Cytoxan), methotrexate (Amethopterin, Mexate, Folex), and fluorouracil (Fluorouracil, 5-FU, Adrucil); (b) Cyclophosphamide, doxorubicin (Adriamycin), and fluorouracil; (c) Doxorubicin (Adriamycin) and cyclophosphamide; (d) Doxorubicin (Adriamycin) and cyclophosphamide with paclitaxel; (e) Doxorubicin (Adriamycin), followed by CMF; (f) Cyclophosphamide, epirubicin, and fluorouracil. Other chemotherapy drugs used for treating women with breast cancer include docetaxel, vinorelbine, gemcitabine, capecitabine, and trastuzumab.

Additionally, several approaches to blocking the effect of estrogen or lowering estrogen levels are used to treat breast cancer. Estrogen is produced mainly by a women's ovaries until menopause, and then by fat tissue, and estrogen promotes the growth of about ⅔ of breast cancers (those containing estrogen or progesterone receptors) and it is therefore a key target in the treatment of breast cancer.

The antiestrogen drug used most often is tamoxifen. It is taken daily in pill form. Taking tamoxifen after surgery, usually for 5 years, can reduce the chances of the cancer coming back if the cancer contained estrogen or progesterone receptors. Tamoxifen is also used to treat metastatic breast cancer. It is used to prevent the development of breast cancer in a woman at high risk, as well. Also contemplated for use in the treatment of breast cancer are raloxifene (Evista), toremifene (Fareston), or fulvestrant (Faslodex).

Further, aromatase inhibitors stop estrogen production in postmenopausal women and they have been approved for use in treating breast cancer. These drugs are letrozole (Femara), anastrozole (Arimidex), and exemestane (Aromasin). They work by blocking an enzyme responsible for producing small amounts of estrogen in postmenopausal women.

(c) Melanoma

Chemotherapy drugs often used to treat melanoma include: (a) Dacarbazine (also called DTIC), alone or in combination with other chemotherapy drugs such as carmustine (also known as BCNU) and cisplatin. The combination of these 3 chemotherapy drugs, together with tamoxifen (a hormonal therapy drug most often used to treat breast cancer) is called the “Dartmouth regimen”; (b) Cisplatin, vinblastine, and DTIC is another chemotherapy combination for treating melanoma; (c) Temozolomide (Temodar) is a drug that works similar to DTIC, but it can be given in the form of a pill.

Immunotherapy enhances and encourages a patient's immune system to recognize and destroy cancer cells more effectively. There are several types of immunotherapy used in treating patients with advanced melanoma. Cytokines are proteins that activate the immune system in a general way. Two cytokines, interferon-alpha and interleukin-2, can help boost immunity in patients with melanoma. Both drugs can help shrink metastatic (stage III and IV) melanomas in about 10% to 20% of patients. Patients with deeper melanomas often have cancer cells that break away from the primary melanoma and travel to other parts of the body. Interferons are immune substances produced by the body in response to infection. Interferon-alpha2b can be used as an adjuvant (added) therapy to prevent the breakaway cells from growing.

(d) Non-Hodgkin's Lymphoma

Cutaneous T-cell Non-Hodgkin's lymphoma is a type of non-Hodgkin's lymphoma (NHL), a cancer of the lymphatic system. CTCL is a rare condition making up about 5% of all cases of NHL. It is a cancer of the T-lymphocytes and most often occurs in people aged between 40 and 60. Unlike other forms of non-Hodgkin's lymphoma, CTCL mainly affects the skin. It is caused by the uncontrolled growth of a type of T-cells in the skin and the causes are unknown. The most common types of CTCL are mycoses fungoides or Sezary syndrome. Sezary syndrome is a specific type of CTCL in which large areas of skin or lymph glands are affected and abnormal T-lymphocytes are also found in the blood. Mycoses fungoides is the general name given to the other types of CTCL when the blood is not affected.

CTCL can be treated in a number of ways, either alone or in combination. Most of the treatments can be used for any stage of disease. The choice of treatment is often made depending on how much of the skin is affected. PUVA treatment is suitable if large areas of the skin are affected. If involves taking psoralens, which sensitizes the skin to the beneficial effects of ultraviolet light. Once the drug has had time to collect in the patient's skin, the patient enters an enclosed air-conditioned cabinet containing UV light. The treatment may be given several times a week.

Usually treatment with skin creams, such as steroids, and moisturizing ointments together with occasional courses of PUVA keeps the lymphoma under control if it is at an early stage. Radiotherapy may be used for early stage disease if only one or two small areas of skin are affected. If necessary, radiotherapy may be given to areas of skin affected by plaques and tumors. Two or three doses of low-dose radiotherapy treatment may be given to the affected area. Radiotherapy may be used to treat the whole skin surface if the lymphoma is more widespread, but has not penetrated below the skin surface. This is called total skin electron beam treatment.

Chemotherapy may be applied in the form of an ointment directly on the whole skin surface or if the condition progresses, intravenous chemotherapy is a treatment option. Interferon may also administered subcutaneously.

Non-Hodgkin's lymphoma is often treated with chlorambucil or fludarabine for low-grade lymphomas, and a combination of cyclophosphamide, doxorubicin, vincristine, and prednisolone, alone or in combination with rituximab, are used for high-grade lymphomas.

### II. Immunostimulatory ODNs

Immunostimulatory ODNs or CpG ODNs contain specific sequences found to elicit an immune response. These specific sequences are referred to as “immunostimulatory motifs”, and the oligonucleotides that contain immunostimulatory motifs are referred to as “immunostimulatory oligonucleotide molecules” and equivalently, “immunostimulatory oligonucleotides”. Immunostimulatory oligonucleotides include at least one immunostimulatory motif and preferably that motif is an internal motif. The term “internal immunostimulatory motif” refers to the position of the motif sequence within an oligonucleotide sequence which is at least one nucleotide longer (at both the 5′ and 3′ ends) than the motif sequence.

CpG oligonucleotides include at least one unmethylated CpG dinucleotide. An oligonucleotide containing at least one unmethylated CpG dinucleotide is a oligonucleotide molecule which contains a cytosine-guanine dinucleotide sequence (i.e., “GpG DNA” or DNA containing a 5′ cytosine linked by a phosphate bond to a 3′ guanine) and activates the immune system. The entire CpG oligonucleotide can be unmethylated or portions may be unmethylated but at least the C of the 5′ CG 3′ must be unmethylated.

The B class of CpG oligonucleotides is represented by at least the formula:

5′X₁X₂CGX₃X₄3′

wherein X₁, X₂, X₃, and X₄ are nucleotides. X₂ may be adenine, guanine, or thymine. X₃ may be cytosine, adenine, or thymine.

The B class of CpG oligonucleotide includes oligonucleotides represented by at least the formula:

5′N₁X₁X₂CGX₃X₄N₂3′

wherein X₁, X₂, X₃, and X₄ are nucleotides and N is any nucleotide and N₁ and N₂ are oligonucleotide sequences composed of from about 0-25 N's each. X₁X₂ may be a dinucleotide selected from the group consisting of: GpT, GpG, GpA, ApA, ApT, ApG, CpT, CpA, CpG, TpA, TpT, and TpG; and X₃X₄ may be a dinucleotide selected from the group consisting of: TpT, ApT, TpG, ApG, CpG, TpC, ApC, CpC, TpA, ApA, and CpA.

The B class of CpG oligonucleotides is disclosed in PCT Published Patent Applications PCT/US95/01570 and PCT/US97/19791, and U.S. Pat. No. 6,194,388 B1 and U.S. Pat. No. 6,239,116 B1, issued Feb. 27, 2001 and May 29, 2001 respectively.

The immunostimulatory oligonucleotide molecules may have a homogeneous backbone (e.g., entirely phosphodiester or entirely phosphorothioate) or a chimeric backbone. For purposes of the instant invention, a chimeric backbone refers to a partially stabilized backbone, wherein at least one internucleotide linkage is phosphodiester or phosphodiester-like, and wherein at least one other internucleotide linkage is a stabilized internucleotide linkage, wherein the at least one phosphodiester or phosphodiester-like linkage and the at least one stabilized linkage are different. Since boranophosphonate linkages have been reported to be stabilized relative to phosphodiester linkages, for purposes of the chimeric nature of the backbone, boranophosphonate linkages can be classified either as phosphodiester-like or as stabilized, depending on the context. For example, a chimeric backbone according to the instant invention could, in one embodiment, include at least one phosphodiester (phosphodiester or phosphodiester-like) linkage and at least one boranophosphonate (stabilized) linkage. In another embodiment, a chimeric backbone according to the instant invention could include boranophosphonate (phosphodiester or phosphodiester-like) and phosphorothioate (stabilized) linkages. A “stabilized internucleotide linkage” shall mean an internucleotide linkage that is relatively resistant to in vivo degradation (e.g., via an exo- or endo-nuclease), compared to a phosphodiester internucleotide linkage. Preferred stabilized internucleotide linkages include, without limitation, phosphorothioate, phosphorodithioate, methylphosphonate and methylphosphorothioate. Other stabilized internucleotide linkages include, without limitation, peptide, alkyl, dephospho type linkages, and others as described above.

Modified backbones such as phosphorothioates may be synthesized using automated techniques employing either phosphoramidate or H-phosphonate chemistries. Aryl- and alkyl-phosphonates can be made, e.g., as described in U.S. Pat. No. 4,469,863; and alkylphosphotriesters (in which the charged oxygen moiety is alkylated), e.g., as described in U.S. Pat. No. 5,023,243 and European Patent No. 092,574, can be prepared by automated solid phase synthesis using commercially available reagents. Methods for making other DNA backbone modifications and substitutions have been described. Uhlmann E et al. (1990) Chem Rev 90:544; Goodchild J (1990) Bioconjugate Chem 1:165. Methods for preparing chimeric oligonucleotides are also known. For instance patents issued to Uhlmann et al. have described such techniques.

Mixed backbone modified ODN may be synthesized using a commercially available DNA synthesizer and standard phosphoramidite chemistry. (F. E. Eckstein, “Oligonucleotides and Analogues—A Practical Approach” IRL Press, Oxford, UK, 1991, and M. D. Matteucci and M. H. Caruthers, Tetrahedron Lett. 21, 719 (1980)) After coupling, PS linkages are introduced by sulfurization using the Beaucage reagent (R. P. Iyer, W. Egan, J. B. Regan and S. L. Beaucage, J. Am. Chem. Soc. 112, 1253 (1990)) (0.075 M in acetonitrile) or phenyl acetyl disulfide (PADS) followed by capping with acetic anhydride, 2,6-lutidine in tetrahydrofurane (1:1:8; v:v:v) and N-methylimidazole (16% in tetrahydrofurane). This capping step is performed after the sulfurization reaction to minimize formation of undesired phosphodiester (PO) linkages at positions where a phosphorothioate linkage should be located. In the case of the introduction of a phosphodiester linkage, e.g. at a CpG dinucleotide, the intermediate phosphorous-III is oxidized by treatment with a solution of iodine in water/pyridine. After cleavage from the solid support and final deprotection by treatment with concentrated ammonia (15 hrs at 50° C.), the ODN are analyzed by HPLC on a Gen-Pak Fax column (Millipore-Waters) using a NaCl-gradient (e.g. buffer A: 10 mM NaH₂PO₄ in acetonitrile/water=1:4/v:v pH 6.8; buffer B: 10 mM NaH₂PO₄, 1.5 M NaCl in acetonitrile/water=1:4/v:v; 5 to 60% B in 30 minutes at 1 ml/min) or by capillary gel electrophoresis. The ODN can be purified by HPLC or by FPLC on a Source High Performance column (Amersham Pharmacia). HPLC-homogeneous fractions are combined and desalted via a C18 column or by ultrafiltration. The ODN was analyzed by MALDI-TOF mass spectrometry to confirm the calculated mass.

The oligonucleotides of the invention can also include other modifications. These include nonionic DNA analogs, such as alkyl- and aryl-phosphates (in which the charged phosphonate oxygen is replaced by an alkyl or aryl group), phosphodiester and alkylphosphotriesters, in which the charged oxygen moiety is alkylated. Oligonucleotides which contain diol, such as tetraethyleneglycol or hexaethyleneglycol, at either or both termini have also been shown to be substantially resistant to nuclease degradation.

In some embodiments the oligonucleotides may be soft or semi-soft oligonucleotides. A soft oligonucleotide is an immunostimulatory oligonucleotide having a partially stabilized backbone, in which phosphodiester or phosphodiester-like internucleotide linkages occur only within and immediately adjacent to at least one internal pyrimidine-purine dinucleotide (YZ). Preferably YZ is YG, a pyrimidine-guanosine (YG) dinucleotide. The at least one internal YZ dinucleotide itself has a phosphodiester or phosphodiester-like internucleotide linkage. A phosphodiester or phosphodiester-like internucleotide linkage occurring immediately adjacent to the at least one internal YZ dinucleotide can be 5′, 3′, or both 5′ and 3′ to the at least one internal YZ dinucleotide.

In particular, phosphodiester or phosphodiester-like internucleotide linkages involve “internal dinucleotides”. An internal dinucleotide in general shall mean any pair of adjacent nucleotides connected by an internucleotide linkage, in which neither nucleotide in the pair of nucleotides is a terminal nucleotide, i.e., neither nucleotide in the pair of nucleotides is a nucleotide defining the 5′ or 3′ end of the oligonucleotide. Thus a linear oligonucleotide that is n nucleotides long has a total of n−1 dinucleotides and only n−3 internal dinucleotides. Each internucleotide linkage in an internal dinucleotide is an internal internucleotide linkage. Thus a linear oligonucleotide that is n nucleotides long has a total of n−1 internucleotide linkages and only n−3 internal internucleotide linkages. The strategically placed phosphodiester or phosphodiester-like internucleotide linkages, therefore, refer to phosphodiester or phosphodiester-like internucleotide linkages positioned between any pair of nucleotides in the oligonucleotide sequence. In some embodiments the phosphodiester or phosphodiester-like internucleotide linkages are not positioned between either pair of nucleotides closest to the 5′ or 3′ end.

Preferably a phosphodiester or phosphodiester-like internucleotide linkage occurring immediately adjacent to the at least one internal YZ dinucleotide is itself an internal internucleotide linkage. Thus for a sequence N₁YZN21 wherein N₁ and N₂ are each, independent of the other, any single nucleotide, the YZ dinucleotide has a phosphodiester or phosphodiester-like internucleotide linkage, and in addition (a) N₁ and Y are linked by a phosphodiester or phosphodiester-like internucleotide linkage when N₁ is an internal nucleotide, (b) Z and N₂ are linked by a phosphodiester or phosphodiester-like internucleotide linkage when N₂ is an internal nucleotide, or (c) N₁ and Y are linked by a phosphodiester or phosphodiester-like internucleotide linkage when N₁ is an internal nucleotide and Z and N₂ are linked by a phosphodiester or phosphodiester-like internucleotide linkage when N₂ is an internal nucleotide.

Soft oligonucleotides according to the instant invention are believed to be relatively susceptible to nuclease cleavage compared to completely stabilized oligonucleotides. Without intending to be bound to a particular theory or mechanism, it is believed that soft oligonucleotides of the invention are susceptible to cleavable resulting in fragments with reduced or no immunostimulatory activity relative to full-length soft oligonucleotides. Incorporation of at least one nuclease-sensitive internucleotide linkage, particularly near the middle of the oligonucleotide, is believed to provide an “off switch” which alters the pharmacokinetics of the oligonucleotide so as to reduce the duration of maximal immunostimulatory activity of the oligonucleotide. This can be of particular value in tissues and in clinical applications in which it is desirable to avoid injury related to chronic local inflammation or immunostimulation, e.g., the kidney.

A semi-soft oligonucleotide is an immunostimulatory oligonucleotide having a partially stabilized backbone, in which phosphodiester or phosphodiester-like internucleotide linkages occur only within at least one internal pyrimidine-purine (YZ) dinucleotide. Semi-soft oligonucleotides generally possess increased immunostimulatory potency relative to corresponding fully stabilized immunostimulatory oligonucleotides. Due to the greater potency of semi-soft oligonucleotides, semi-soft oligonucleotides may be used, in some instances, at lower effective concentrations and have lower effective doses than conventional fully stabilized immunostimulatory oligonucleotides in order to achieve a desired biological effect.

It is believed that the foregoing properties of semi-soft oligonucleotides generally increase with increasing “dose” of phosphodiester or phosphodiester-like internucleotide linkages involving internal YZ dinucleotides. Thus it is believed, for example, that generally for a given oligonucleotide sequence with four internal YZ dinucleotides, an oligonucleotide with four internal phosphodiester or phosphodiester-like YZ internucleotide linkages is more immunostimulatory than an oligonucleotide with three internal phosphodiester or phosphodiester-like YZ internucleotide linkages, which in turn is more immunostimulatory than an oligonucleotide with two internal phosphodiester or phosphodiester-like YZ internucleotide linkages, which in turn is more immunostimulatory than an oligonucleotide with one internal phosphodiester or phosphodiester-like YZ internucleotide linkage. Importantly, inclusion of even one internal phosphodiester or phosphodiester-like YZ internucleotide linkage is believed to be advantageous over no internal phosphodiester or phosphodiester-like YZ internucleotide linkage. In addition to the number of phosphodiester or phosphodiester-like internucleotide linkages, the position along the length of the oligonucleotide can also affect potency.

The soft and semi-soft oligonucleotides will generally include, in addition to the phosphodiester or phosphodiester-like internucleotide linkages at preferred internal positions, 5′ and 31 ends that are resistant to degradation. Such degradation-resistant ends can involve any suitable modification that results in an increased resistance against exonuclease digestion over corresponding unmodified ends. For instance, the 5′ and 3′ ends can be stabilized by the inclusion there of at least one phosphate modification of the backbone. In a preferred embodiment, the at least one phosphate modification of the backbone at each end is independently a phosphorothioate, phosphorodithioate, methylphosphonate, or methylphosphorothioate internucleotide linkage. In another embodiment, the degradation-resistant end includes one or more nucleotide units connected by peptide or amide linkages at the 3′ end.

A phosphodiester internucleotide linkage is the type of linkage characteristic of oligonucleotides found in nature. The phosphodiester internucleotide linkage includes a phosphorus atom flanked by two bridging oxygen atoms and bound also by two additional oxygen atoms, one charged and the other uncharged. Phosphodiester internucleotide linkage is particularly preferred when it is important to reduce the tissue half-life of the oligonucleotide.

A phosphodiester-like internucleotide linkage is a phosphorus-containing bridging group that is chemically and/or diastereomerically similar to phosphodiester. Measures of similarity to phosphodiester include susceptibility to nuclease digestion and ability to activate RNase H. Thus, for example phosphodiester, but not phosphorothioate, oligonucleotides are susceptible to nuclease digestion, while both phosphodiester and phosphorothioate oligonucleotides activate RNAse H. In a preferred embodiment the phosphodiester-like internucleotide linkage is boranophosphate (or equivalently, boranophosphonate) linkage. U.S. Pat. No. 5,177,198; U.S. Pat. No. 5,859,231; U.S. Pat. No. 6,160,109; U.S. Pat. No. 6,207,819; Sergueev et al., (1998) J Am Chem Soc 120:9417-27. In another preferred embodiment the phosphodiester-like internucleotide linkage is diastereomerically pure Rp phosphorothioate. It is believed that diastereomerically pure Rp phosphorothioate is more susceptible to nuclease digestion and is better at activating RNAse H than mixed or diastereomerically pure Sp phosphorothioate. Stereoisomers of CpG oligonucleotides are the subject of published PCT application PCT/US99/17100 (WO 00/06588). It is to be noted that for purposes of the instant invention, the term “phosphodiester-like internucleotide linkage” specifically excludes phosphorodithioate and methylphosphonate internucleotide linkages.

As described above the soft and semi-soft oligonucleotides of the invention may have phosphodiester like linkages between C and G. One example of a phosphodiester-like linkage is a phosphorothioate linkage in an Rp conformation. Oligonucleotide p-chirality can have apparently opposite effects on the immune activity of a CpG oligonucleotide, depending upon the time point at which activity is measured. At an early time point of 40 minutes, the Rₚ but not the S_{P} stereoisomer of phosphorothioate CpG oligonucleotide induces JNK phosphorylation in mouse spleen cells. In contrast, when assayed at a late time point of 44 hr, the S_{P} but not the Rₚ stereoisomer is active in stimulating spleen cell proliferation. This difference in the kinetics and bioactivity of the Rₚ and S_{P} stereoisomers does not result from any difference in cell uptake, but rather most likely is due to two opposing biologic roles of the p-chirality. First, the enhanced activity of the Rp stereoisomer compared to the Sp for stimulating immune cells at early time points indicates that the Rp may be more effective at interacting with the CpG receptor, TLR9, or inducing the downstream signaling pathways. On the other hand, the faster degradation of the Rp PS-oligonucleotides compared to the Sp results in a much shorter duration of signaling, so that the Sp PS-oligonucleotides appear to be more biologically active when tested at later time points.

A surprisingly strong effect is achieved by the p-chirality at the CpG dinucleotide itself. In comparison to a stereo-random CpG oligonucleotide the congener in which the single CpG dinucleotide was linked in Rp was slightly more active, while the congener containing an Sp linkage was nearly inactive for inducing spleen cell proliferation.

Thus the oligonucleotides may be heterogeneous in backbone composition thereby containing any possible combination of polymer units linked together.

The term “oligonucleotide” also encompasses oligonucleotides with substitutions or modifications, such as in the sugars. For example, they include oligonucleotides having backbone sugars that are covalently attached to low molecular weight organic groups other than a hydroxyl group at the 2′ position and other than a phosphate group or hydroxy group at the 5′ position. Thus modified oligonucleotides may include a 2′-O-alkylated ribose group. In addition, modified oligonucleotides may include sugars such as arabinose or 2′-fluoroarabinose instead of ribose.

The immunostimulatory oligonucleotides of the instant invention can encompass various chemical modifications and substitutions, in comparison to natural RNA and DNA, involving a phosphodiester internucleotide bridge, or a β-D-ribose unit. Examples of chemical modifications are known to the skilled person and are described, for example, in Uhlmann E et al. (1990) Chem Rev 90:543; “Protocols for Oligonucleotides and Analogs” Synthesis and Properties & Synthesis and Analytical Techniques, S. Agrawal, Ed, Humana Press, Totowa, USA 1993; Crooke S T et al., (1996) Annu Rev Pharmacol Toxicol 36:107-129; and Hunziker J et al. (1995) Mod Synth Methods 7:331-417. An oligonucleotide according to the invention may have one or more modifications, wherein each modification is located at a particular phosphodiester internucleotide bridge and/or at a particular β-D-ribose unit in comparison to an oligonucleotide of the same sequence which is composed of natural DNA or RNA.

For example, the invention relates to an oligonucleotide which may comprise one or more modifications and wherein each modification is independently selected from: (a) the replacement of a phosphodiester internucleotide bridge located at the 3′ and/or the 5′ end of a nucleotide by a modified internucleotide bridge; (b) the replacement of phosphodiester bridge located at the 3′ and/or the 5′ end of a nucleotide by a dephospho bridge; (c) the replacement of a sugar phosphate unit from the sugar phosphate backbone by another unit; and (d) the replacement of a β-D-ribose unit by a modified sugar unit. More detailed examples for the chemical modification of an oligonucleotide are as follows:

A phosphodiester internucleotide bridge located at the 3′ and/or the 5′ end of a nucleotide can be replaced by a modified internucleotide bridge, wherein the modified internucleotide bridge is for example selected from phosphorothioate, phosphorodithioate, NR¹R²-phosphoramidate, boranophosphate, α-hydroxybenzyl phosphonate, phosphate-(C₁-C₂₁)—O-alkyl ester, phosphate-[(C₆-C₁₂)aryl-(C₁-C₂₁)-□-alkyl]ester, (C₁-C₈)alkylphosphonate and/or (C₆-C₁₂)arylphosphonate bridges, (C₇-C₁₂)-□-hydroxymethyl-aryl (e.g., disclosed in WO 95/01363), wherein (C₆-C₁₂)aryl, (C₆-C₂₀)aryl and (C₆-C₁₄)aryl are optionally substituted by halogen, alkyl, alkoxy, nitro, cyano, and where R¹ and R² are, independently of each other, hydrogen, (C₁-C₁₈)-alkyl, (C₁-C₂₀)-aryl, (C₆-C₁₄)-aryl-(C₁-C₈)-alkyl, preferably hydrogen, (C₁-C₈)-alkyl, preferably (C₁-C₄)-alkyl and/or methoxyethyl, or R¹ and R² form, together with the nitrogen atom carrying them, a 5-6-membered heterocyclic ring which can additionally contain a further heteroatom from the group O, S and N.

The replacement of a phosphodiester bridge located at the 3′ and/or the 5′ end of a nucleotide by a dephospho bridge (dephospho bridges are described, for example, in Uhlmann E and Peyman A in “Methods in Molecular Biology”, Vol. 20, “Protocols for Oligonucleotides and Analogs”, S. Agrawal, Ed., Humana Press, Totowa 1993, Chapter 16, pp. 355 ff), wherein a dephospho bridge is for example selected from the dephospho bridges formacetal, 3′-thioformacetal, methylhydroxylamine, oxime, methylenedimethyl-hydrazo, dimethylenesulfone and/or silyl groups.

A sugar phosphate unit (i.e., a β-D-ribose and phosphodiester internucleotide bridge together forming a sugar phosphate unit) from the sugar phosphate backbone (i.e., a sugar phosphate backbone is composed of sugar phosphate units) can be replaced by another unit, wherein the other unit is for example suitable to build up a “morpholino-derivative” oligomer (as described, for example, in Stirchak E P et al. (1989) Oligonucleotides Res 17:6129-41), that is, e.g., the replacement by a morpholino-derivative unit; or to build up a polyamide oligonucleotide (“PNA”; as described for example, in Nielsen P E et al. (1994) Bioconjug Chem 5:3-7), that is, e.g., the replacement by a PNA backbone unit, e.g., by 2-aminoethylglycine.

A β-ribose unit or a β-D-2′-deoxyribose unit can be replaced by a modified sugar unit, wherein the modified sugar unit is for example selected from β-D-ribose, α-D-2′-deoxyribose, L-2′-deoxyribose, 2′-F-2′-deoxyribose, 2′-F-arabinose, 2′-O—(C₁-C₆)alkyl-ribose, preferably 2′-O—(C₁-C₆)alkyl-ribose is 2′-O-methylribose, 2′-O—(C₂-C₆)alkenyl-ribose, 2′-[O—(C₁-C₆)alkyl-O—(C₁-C₆)alkyl]-ribose, 2′-NH₂-2′-deoxyribose, β-D-xylo-furanose, α-arabinofuranose, 2,4-dideoxy-β-D-erythro-hexo-pyranose, and carbocyclic (described, for example, in Froehler J (1992) Am Chem Soc 114:8320) and/or open-chain sugar analogs (described, for example, in Vandendriessche et al. (1993) Tetrahedron 49:7223) and/or bicyclosugar analogs (described, for example, in Tarkov M et al. (1993) Helv Chim Acta 76:481).

In some embodiments the sugar is 2′-O-methylribose, particularly for one or both nucleotides linked by a phosphodiester or phosphodiester-like internucleotide linkage.

In particular sequences described herein a set of modified bases is defined. For instance the letter Y is used to refer to a nucleotide containing a cytosine or a modified cytosine. A modified cytosine as used herein is a naturally occurring or non-naturally occurring pyrimidine base analog of cytosine which can replace this base without impairing the immunostimulatory activity of the oligonucleotide. Modified cytosines include but are not limited to 5-substituted cytosines (e.g. 5-methyl-cytosine, 5-fluoro-cytosine, 5-chloro-cytosine, 5-bromo-cytosine, 5-Iodo-cytosine, 5-hydroxy-cytosine, 5-hydroxymethyl-cytosine, 5-difluoromethyl-cytosine, and unsubstituted or substituted 5-alkynyl-cytosine), 6-substituted cytosines, N4-substituted cytosines (e.g. N4-ethyl-cytosine), 5-aza-cytosine, 2-mercapto-cytosine, isocytosine, pseudo-isocytosine, cytosine analogs with condensed ring systems (e.g. N,N′-propylene cytosine or phenoxazine), and uracil and its derivatives (e.g. 5-fluoro-uracil, 5-bromo-uracil, 5-bromovinyl-uracil, 4-thio-uracil, 5-hydroxy-uracil, 5-propynyl-uracil). Some of the preferred cytosines include 5-methyl-cytosine, 5-fluoro-cytosine, 5-hydroxy-cytosine, 5-hydroxymethyl-cytosine, and N4-ethyl-cytosine. In another embodiment of the invention, the cytosine base is substituted by a universal base (e.g. 3-nitropyrrole, P-base), an aromatic ring system (e.g. fluorobenzene or difluorobenzene) or a hydrogen atom (dSpacer).

The letter Z is used to refer to guanine or a modified guanine base. A modified guanine as used herein is a naturally occurring or non-naturally occurring purine base analog of guanine which can replace this base without impairing the immunostimulatory activity of the oligonucleotide. Modified guanines include but are not limited to 7-deazaguanine, 7-deaza-7-substituted guanine (such as 7-deaza-7-(C₂-C₆)alkynylguanine), 7-deaza-8-substituted guanine, hypoxanthine, N2-substituted guanines (e.g. N2-methylguanine), 5-amino-3-methyl-3H,6H-thiazolo[4,5-d]pyrimidine-2,7-dione, 2,6-diaminopurine, 2-aminopurine, purine, indole, adenine, substituted adenines (e.g. N6-methyl-adenine, 8-oxoadenine) 8-substituted guanine (e.g. 8-hydroxyguanine and 8-bromoguanine), and 6-thioguanine. In another embodiment of the invention, the guanine base is substituted by a universal base (e.g. 4-methyl-indole, 5-nitro-indole, and K-base), an aromatic ring system (e.g. benzimidazole or dichloro-benzimidazole, 1-methyl-1H-[1,2,4]triazole-3-carboxylic acid amide) or a hydrogen atom (dSpacer).

The oligonucleotides may have one or more accessible 5′ ends. It is possible to create modified oligonucleotides having two such 5′ ends. This may be achieved, for instance by attaching two oligonucleotides through a 3′-3′ linkage to generate an oligonucleotide having one or two accessible 5′ ends. The 3′-3′-linkage may be a phosphodiester, phosphorothioate or any other modified internucleotide bridge. Methods for accomplishing such linkages are known in the art. For instance, such linkages have been described in Seliger, H.; et al., Oligonucleotide analogs with terminal 3′-3′- and 5′-5′-internucleotidic linkages as antisense inhibitors of viral gene expression, Nucleotides & Nucleotides (1991), 10(1-3), 469-77 and Jiang, et al., Pseudo-cyclic oligonucleotides: in vitro and in vivo properties, Bioorganic & Medicinal Chemistry (1999), 7(12), 2727-2735.

Additionally, 3′3′-linked oligonucleotides where the linkage between the 3′-terminal nucleotides is not a phosphodiester, phosphorothioate or other modified bridge, can be prepared using an additional spacer, such as tri- or tetra-ethyleneglycol phosphate moiety (Durand, M. et al, Triple-helix formation by an oligonucleotide containing one (dA)12 and two (dT)12 sequences bridged by two hexaethylene glycol chains, Biochemistry (1992), 31(38), 9197-204, U.S. Pat. No. 5,658,738, and U.S. Pat. No. 5,668,265). Alternatively, the non-nucleotidic linker may be derived from ethanediol, propanediol, or from an abasic deoxyribose (dSpacer) unit (Fontanel, Marie Laurence et al., Sterical recognition by T4 polynucleotide kinase of non-nucleosidic moieties 5′-attached to oligonucleotides; Oligonucleotides Research (1994), 22(11), 2022-7) using standard phosphoramidite chemistry. The non-nucleotidic linkers can be incorporated once or multiple times, or combined with each other allowing for any desirable distance between the 3′-ends of the two ODNs to be linked.

The oligonucleotides are partially resistant to degradation (e.g., are stabilized). A “stabilized oligonucleotide molecule” shall mean an oligonucleotide that is relatively resistant to in vivo degradation (e.g. via an exo- or endo-nuclease). Oligonucleotide stabilization can be accomplished via backbone modifications. Oligonucleotides having phosphorothioate linkages provide maximal activity and protect the oligonucleotide from degradation by intracellular exo- and endo-nucleases. Other modified oligonucleotides include phosphodiester modified oligonucleotides, combinations of phosphodiester and phosphorothioate oligonucleotide, methyl phosphonate, methyl phosphorothioate, phosphorodithioate, p-ethoxy, and combinations thereof. Oligonucleotides which contain diol, such as tetraethyleneglycol or hexaethyleneglycol, at either or both termini have also been shown to be substantially resistant to nuclease degradation.

The immunostimulatory oligonucleotides may also contain one or more unusual linkages between the nucleotide or nucleotide-analogous moieties. The usual internucleoside linkage is a 3′5′-linkage. All other linkages are considered to be unusual internucleoside linkages, such as 2′5′-, 5′6′-, 3′3′-, 2′2′-, 2′3′-linkages. The nomenclature 2′ to 5′ is chosen according to the carbon atom of ribose. However, if unnatural sugar moieties are employed, such as ring-expanded sugar analogs (e.g. hexanose, cyclohexene or pyranose) or bi- or tricyclic sugar analogs, then this nomenclature changes according to the nomenclature of the monomer. In 3′-deoxy-β-D-ribopyranose analogs (also called p-DNA), the mononucleotides are e.g. connected via a 4′2-linkage.

If the oligonucleotide contains one 3′3′-linkage, then this oligonucleotide may have two unlinked 5′-ends. Similarly, if the oligonucleotide contains one 5′5′-linkage, then this oligonucleotide may have two unlinked 3′-ends. The accessibility of unlinked ends of nucleotides may be better accessible by their receptors. Both types of unusual linkages (3′3′- and 5′5′) were described by Ramalho Ortigao et al. (Antisense Research and Development (1992) 2, 129-46), whereby oligonucleotides having a 3′3′-linkage were reported to show enhanced stability towards cleavage by nucleases.

Different types of linkages can also be combined in one molecule which may lead to branching of the oligomer. If one part of the oligonucleotide is connected at the 3′-end via a 3′3′-linkage to a second oligonucleotide part and at the 2′-end via a 2′3′-linkage to a third part of the molecule, this results e.g. in a branched oligonucleotide with three 5′-ends (3′3′-, 2′3′-branched). The variations in linkages include, without limitation, the following:

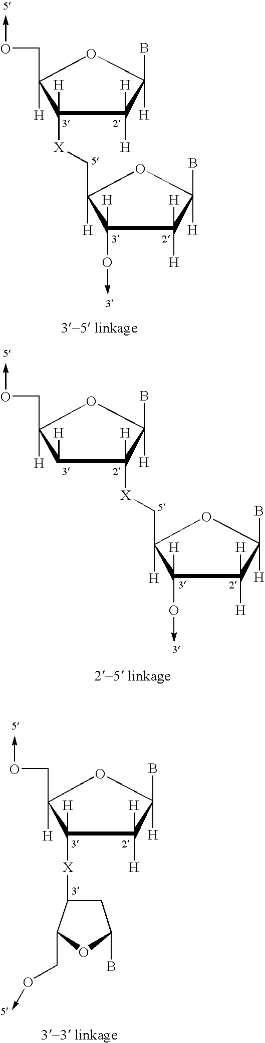

wherein X includes, without limitation:

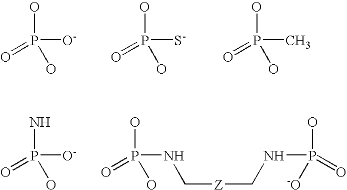

Alternatively, the following variations are also contemplated:

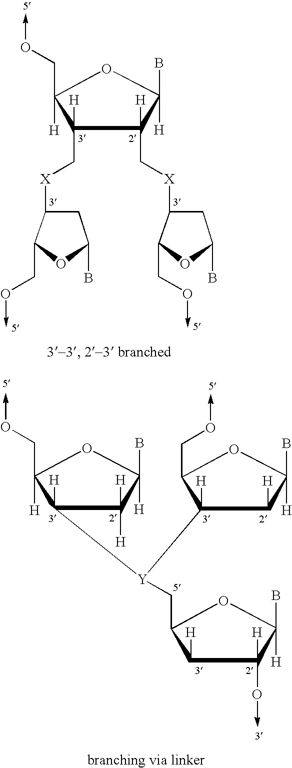

X includes but is not limited to:

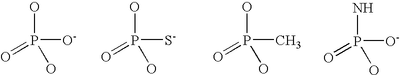

Y includes but is not limited to:

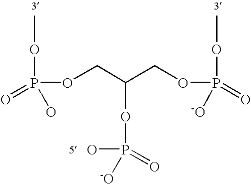

PF-3512676 is a Toll-Like Receptor-9 (TLR-9) agonist that, by activation of plasmacytoid dendritic cells and B-lymphocytes, provides a targeted and specific modality of immunotherapy that has potential utility as an anticancer therapy. PF-3512676 is an oligodeoxynucleotide (ODN) optimized for specific binding to TLR-9 to produce potent activation of innate and adaptive immune responses. PF-3512676 is not an antisense ODN for gene therapy and is non-genotoxic.

PF-3512676 is a synthetic single stranded oligodeoxynucleotide (ODN) molecule, twenty-four nucleotides in length with natural DNA bases and sugars on a nuclease-resistant phosphorothioate backbone that provides in vivo resistance to degradation. PF-3512676 belongs to a group of ODNs characterized by repeated sequences of cytosines and guanines (CpG motifs). These CpG motifs are known to induce immune activation and PF-3512676 has a nucleotide sequence optimized for potent immunomodulation.

The immune system has evolved to detect and recognize pathogen-associated molecular patterns and then induce appropriate Th1 or Th2 immune responses with initial innate and subsequent adaptive (antigen-specific) immune activation. Members of the toll-like receptor (TLR) family have emerged as key regulators of both innate and adaptive immune responses. The molecular patterns recognized by TLR-9 as pathogen-associated are unmethylated CpG dinucleotides in specific sequence contexts (CpG motifs). These CpG motifs are common in bacterial or viral DNA, but underrepresented and methylated in vertebrate DNA. TLR-9 is expressed on plasmacytoid dendritic cells (pDC) and B-lymphocytes and DNA with CpG motifs induces potent TLR-9 mediated innate and adaptive Th1 and to a lesser extent Th2 immune responses.

Selective TLR-9 mediated immune activation can be mimicked by synthetic ODNs containing CpG motifs and PF-3512676 has a nucleotide sequence optimized for ligand agonism of human TLR-9. PF-3512676 can therefore provide a targeted and specific modality of immunotherapy, inducing immune responses through activation of plasmacytoid dendritic cells (pDC) and B-lymphocytes with subsequent stimulation of lymphocyte, macrophage, monocyte, natural killer (NK) and T cell populations.

The exact contribution of innate and adaptive immune responses to PF-3512676-based cancer immunotherapy remains under investigation. However, preclinical studies suggest that a number of different immune based anti-tumor mechanisms may be elicited, including (a) activation of innate immune cells such as NK cells and monocytes that may target tumor cells; (b) induction of interferons (INFs) and interferon inducible gene products such as IP-10 that may have direct antitumor effects; (c) improved presentation of tumor-specific antigens to T-cells, stimulating the development of tumor-directed cytotoxic T lymphocytes; and/or (d) breaking immune tolerance to tumor-specific antigens and inducing maturation of naïve tumor-specific T cells. Based on the mechanism of action and supportive preclinical pharmacology data, PF-3512676 provides a targeted and specific modality of immunotherapy that can be administered alone or as part of combination anti-cancer therapy.

HYB2055 for Injection for cancer treatment is referred to as IMOxine®₁ which is produced by Idera Inc., formerly Hybridon Inc., Cambridge, Mass. IMOxine® is a second-generation IMO™ agonist for TLR9 containing a CpR™ dinucleotide motif (wherein R denotes a 7-aza-guanosine) and novel DNA structure. In human cell based assays, IMOxine® showed activation of B cells and dendritic cells and induced Th1-type cytokine profiles. In preclinical animal models, IMOxine® has shown the following immunostimulatory profiles which may be appropriate for cancer treatment: (a) stimulated Th1 immune responses; (b) produced therapeutic cytokines IL-12, IFN-α/γ; (c) activated NK cells; (d) induced CTL responses; (e) promoted tumor-specific memory responses; and/or (f) potentiated antitumor activity of selected chemotherapeutic agents, monoclonal antibodies, vaccines and antigens.

As to IMOxine®, reference is made to Agrawal et al., WO 98/49288, published Nov. 5, 1998; WO 01/12804, published Feb. 22, 2001; WO 01/55370, published Aug. 2, 2001; PCT/US01/13682, filed Apr. 30, 2001; and PCT/US01/30137, filed Sep. 26, 2001; US2005/0009773, filed May 14, 2004. Reference is also made to the following U.S. applications and issued U.S. patents, the disclosures of which are hereby incorporated herein by reference: US20050130918, US20050054600, US20050009773, US20040198685, US20040156825, U.S. Pat. No. 6,815,429, US20040106570, US20040097719, U520040033980, U.S. Pat. No. 6,667,293, U.S. Pat. No. 6,649,596, U.S. Pat. No. 6,645,943, U.S. Pat. No. 6,624,293, U.S. Pat. No. 6,608,035, U.S. Pat. No. 6,605,708, US20030191078, US20030186911, US20030125287, US20030109479, US20030032612, U.S. Pat. No. 6,531,589, U.S. Pat. No. 6,509,459, U.S. Pat. No. 6,509,149,U.S. Pat. No. 6,489,464, U.S. Pat. No. 6,489,304, U.S. Pat. No. 6,485,973, U.S. Pat. No. 6,476,000, U.S. Pat. No. 6,458,940, U.S. Pat. No. 6,440,660, U.S. Pat. No. 6,426,334, U.S. Pat. No. 6,399,586, U.S. Pat. No. 6,383,752, U.S. Pat. No. 6,372,427, U.S. Pat. No. 6,346,614, U.S. Pat. No. 6,335,436, and U.S. Pat. No. 6,306,829.

Thus, in a preferred embodiment the CpG ODN is IMOxine® and it is administered with one or more chemotherapeutic agent(s) at a dosage of about 0.001 mg/kg to about 10 mg/kg. In a preferred embodiment, IMOxine® is administered at a dosage of about 0.01 mg/kg to about 1 mg/kg, and IMOxine® is preferably administered subcutaneously.

1018 ISS (provided by Dynavax Technologies Corporation, Berkeley, Calif.) is a single stranded, 22 base immunostimulatory phosphorothioate oligonucleotide prepared by standard solid-phase chemistry techniques (sequence 5′ TGACTGTGAACGTTCGAGA TGA 3′ (SEQ ID NO:2)) with a molecular mass of approximately 7150 daltons. With respect to 1018 ISS, reference is made to U.S. Pat. No. 5,589,940 and U.S. Pat. No. 6,225,292, each of which are incorporated herein by reference. Reference is also made to the following U.S. patent applications and issued U.S. patents, the disclosures of which are hereby incorporated herein by reference: U.S. Pat. No. 6,426,336, U.S. Pat. No. 6,174,872, U.S. Pat. No. 5,849,719, U.S. Pat. No. 5,679,647, U.S. Pat. No. 6,498,148, U.S. Pat. No. 6,514,948, U.S. Pat. No. 6,534,062, U.S. Pat. No. 6,552,006, U.S. Pat. No. 6,562,798, U.S. Pat. No. 6,589,940, U.S. Pat. No. 6,610,661, U.S. Pat. No. 6,613,751 US20030027782, US20020086839, US20030092663, US20030078223, US20030119773, US20030143213, US20030147870, US20020142977, US20030212028, US20040092468, US20040006034, US20020086839, US20040006010, US20030232780, US20020042387, US20030130217, US20030176389, US20030203861, US20030109469, US20030186921, US20030064064, US20020028784, US20020098199, US20030216340, US20010046967, US20040009942, US20020107212, US20020055477, US20030022852, US20030059773, US20030125284, US20030129251, US20030176373, US20020142978, US20030049266, US20030078223, US20030092663, US20030133988, US20030175731, US20030225016, US20030199466, and US20040132677.

In some embodiments, the CpG ODN is 1018 ISS and it is administered with one or more chemotherapeutic agent(s) at a dosage of about 0.001 mg/kg to about 10 mg/kg. In a preferred embodiment, 1018 ISS is administered at a dosage of about 0.01 mg/kg to about 1 mg/kg, and in a most preferred embodiment, 1018 ISS is administered at a dosage of about 0.01 mg/kg to about 0.5 mg/kg. Finally, 1018 ISS is preferably administered subcutaneously.

Another approach in the development of CpG ODNs that modulate the TLR9 receptor is the development of immune modulators based on DNA called dSLIMs, or double stem-loop immune modulators. Such molecules have a dumbbell shape consisting of a double stranded, 20 base pair piece in the middle and two single-stranded loops of 30 nucleotides each at both ends. Such molecules contain CpG motifs and they trigger a variety of immune modulators such as interferon alpha, gamma, IL-6, and IL-12, but the molecule is covalently closed, so it is protected against single-stranded DNA binding and exonuclease digestion, without the need for stabilization by phosphorothioate groups or the like. U.S. Pat. No. 6,849,725 describes dSLIM technology, and the disclosure of this patent is incorporated herein by reference.

Finally, Genasense® is a drug that is produced by Genta Incorporated, Berkeley Heights, N.J., which inhibits the production of a protein known as Bcl-2. By reducing production of Bcl-2 in cancer cells, Genasense treatment seeks to restore the basic biological process whereby cancer cells can be readily killed by treatment with current methods of anticancer therapy. This process is known as programmed cell death or apoptosis. Genasense is typically administered prior to the administration of anticancer therapy with the intention of potentiating the cancer-killing effects of treatment. Bcl-2 is widely expressed in many types of cancer, and as such, Genasense is potentially applicable to the treatment of an array of tumor types, including and without limitation, melanoma, multiple myeloma, acute myeloid leukemia, chronic lymphocytic leukemia, prostate cancer, and lung cancer. It also has the potential to act synergistically with a variety of chemotherapeutic agents and therapeutic modalities in the treatment of cancer, such as chemotherapy, radiation, monoclonal antibodies and immunotherapy. Based on preclinical data, Genasense may be used in combination with paclitaxel, Irinotecan, Imatinib, rituximab, fludarabine, cyclophosphamide, docetaxel, Mylotarg® (Gemtuzumab ozogamicin; Wyeth-Ayerst, Inc.), Cytosine arabinoside, Dexamethasone, and potentially combinations thereof. Reference is made to U.S. Pat. Nos. 6,262,036, 6,060,545, 5,986,083, 5,854,410, and 5,783,683, the disclosures of which are incorporated herein by reference.

### III. Chemotherapeutic Agents

Each of the chemotherapeutic agents described below are intended to be used in combination with a CpG ODN according to the following treatment guidelines:

A CpG ODN may be administered before, simultaneously, substantially simultaneously, or after (or any combination thereof) administration of the chemotherapeutic agent or combination of chemotherapeutic agents. The CpG ODN may be administered daily during a treatment regimen or maintenance period (including one or more administrations per day), every other day, every three days, every four days, every five days, every six days, or every week, every month, every two months, every three months every four months, every five months, every six months, or every year. A single dose or multiples doses of the chemotherapeutic agent or combination of chemotherapeutic agents may be administered. Alternatively, at least one dose, or at least three, six or 12 doses may be administered. The administration of the ODN and the chemotherapeutic agent or combination of chemotherapeutic agents may alternate.

In one embodiment, the chemotherapeutic agent or combination of chemotherapeutic agents is administered first to block the inhibitory effects that would limit the efficacy of the CpG ODN. In this embodiment, the chemotherapeutic agent or combination of chemotherapeutic agents is given preferably from 1 month to 1 day prior to the CpG ODN. In a preferred embodiment, the chemotherapeutic agent or combination of chemotherapeutic agents is administered at least 1 week but no more than 3 weeks prior to the CpG ODN, and most preferably the chemotherapeutic agent or chemotherapeutic combination of agents is administered about 1 week prior to the administration of CpG ODN. In another embodiment, the CpG ODN is given first, to prime the immune system to have a better immune activation response to the chemotherapeutic agent or combination of chemotherapeutic agents or other therapy that may be given in conjunction with this (e.g., tumor vaccine, surgery, radiation, combinations thereof, etc.). In this embodiment, the CpG ODN is given preferably from 1 month to 1 day prior to the chemotherapeutic agent or combination of chemotherapeutic agents. In a preferred embodiment, the CpG ODN is administered at least 1 week but no more than 3 weeks prior to the chemotherapeutic agent or combination of chemotherapeutic agents, and most preferably the CpG ODN is administered about 1 week prior to the administration of the chemotherapeutic agent or combination of chemotherapeutic agents.

The chemotherapeutic agent or combination of chemotherapeutic agents may be administered with the CpG ODN in a multi-day or multi-week cycle or therapeutic regimen. The multi-day cycle may be a 2, 3, 4, 5, 6, 7, 8, 9, 10 or more day cycle, or a 2, 3, 4 or more week cycle. The chemotherapeutic agent or combination of chemotherapeutic agents may be administered on the first day of such a cycle, followed by administration of the CpG ODN on the first day of each week of a multiweek cycle. For example, the CpG ODN may be administered on days 1, 7 and 14 of a three week cycle. The three week cycle may be repeated once, two three times or more. The entire treatment may be preceded by administration of either the ODN or the chemotherapeutic agent or combination of chemotherapeutic agents alone, for example in order to prime the immune system or render the subject more responsive to the subsequent therapy.

(a) The compound 5-(5-fluoro-2-oxo-1,2-dihydroindol-(3Z)-ylidenemethyl)-2,4-dimethyl-1H-pyrrole-3-carboxylic acid (2-diethylaminoethyl)-amide, represented by formula 1

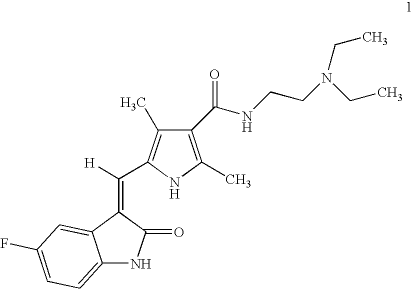

is a novel oral cancer drug shown to have efficacy in a variety of solid tumor types. Compound 1, supplied by Pfizer, Inc., La Jolla, Calif., targets multiple receptor tyrosine kinase inhibitors, including PDGFR, KIT and VEGFR, and is a potent and selective anti-angiogenesis agent. Compound 1 or its L-malate salt is also referred to as SU11248, SU011248, sunitinib malate (USAN/WHO designation) or SUTENT™ (L-malate salt).

The compound, its synthesis, and particular polymorphs are described in U.S. Pat. No. 6,573,293, U.S. Patent Publication Nos. 2003-0229229, 2003-0069298 and 2005-0059824, and in J. M. Manley, M. J. Kalman, B. G. Conway, C. C. Ball, J. L. Havens and R. Vaidyanathan, “Early Amidation Approach to 3-[(4-amido)pyrrol-2-yl]-2-indolinones,” J. Org. Chem. 68, 6447-6450 (2003). Preferred formulations of Compound 1 and its L-malate salt are described in PCT Publication No. WO 2004/024127. Preferred dosing regimens are described in U.S. patent application Ser. No. 10/991,244, filed Nov. 17, 2004, entitled “Method of Treating Abnormal Cell Growth Using Indolinone Compounds. The disclosures of these references are incorporated herein by reference in their entireties. Several references describe combinations of Compound 1 with other agents, e.g. U.S. Patent Publication No. 2003-0216410, U.S. Patent Publication No. 2004-0152759, and U.S. Provisional Application Ser. No. 60/660,624, filed Mar. 11, 2005. The disclosures of these references are incorporated herein by reference in their entireties.

As used herein, unless otherwise indicated, the term “Compound 1” refers to the compound of structural formula 1, also designated as 5-(5-fluoro-2-oxo-1,2-dihydroindol-(3Z)-ylidenemethyl)-2,4-dim ethyl-1H-pyrrole-3-carboxylic acid (2-diethylaminoethyl)-amide, in its free base form, as well as pharmaceutically acceptable salts or solvates (including hydrates) thereof. A particularly preferred salt is a malate salt, more preferably the L-malate salt. References to amounts of Compound 1 refer to free base equivalent amounts. Compound 1 is conveniently provided as an oral dosage form, such as a tablet or capsule, in dosage amounts of 12.5, 25 or 50 mg, free base equivalent. These dosage amounts permit easy dosing adjustments in 12.5 mg increments. Although other dosage amounts are possible, typical dosing ranges are from 12.5 to 75 mg per day, and more typically 25, 37.5, 50 or 62.5 mg per day, free base equivalent. The daily dose is generally taken at a frequency of once per day, without regard to food; i.e., in a fed or fasted state. Compound 1 can be administered in a continuous dosing regimen, i.e., daily for the duration of the treatment period, or in an intermittent dosing regimen, i.e., administered daily during a treatment period, followed by a rest or non-treatment period during which no Compound 1 is administered. In an intermittent dosing regimen, the treatment period is typically from 10 to 30 days, such as 2, 3 or 4 weeks, and the rest period is typically from 3 to 15 days, such as 1 or 2 weeks. The combination of any treatment period from 10 to 30 days with any rest period from 3 to 15 days is contemplated. Several intermittent regimens are presently preferred; expressed as treatment period in weeks/rest period in weeks, preferred regimens include 4/2, 4/1, 3/2, 3/1 and 2/1. It should be further appreciated that dosing regimens can be adjusted by one skilled in the art to more conveniently accommodate coordination of the dosing regimens of Compound 1 and additional therapeutic agents, if such adjustments are therapeutically acceptable.

In a particular embodiment, the invention provides a method of treating or preventing any of the above-recited cancers in a patient, such as a human, by administering to the patient Compound 1 in an amount of up to about 75 mg, e.g., about 25 to 75 mg, preferably 37.5, 50 or 62.5 mg, daily, in combination with up to about 5 mg/kg CpG ODN, preferably PF3512676, 1018 ISS, Genasense or IMOxine®, administered subcutaneously. In a preferred embodiment, Compound 1 is administered on a continuous (i.e., not intermittent) dosing schedule. In a further preferred embodiment, PF3512676 is administered at a therapeutic dose of about 0.01 to 2.5 mg/kg, more preferably, about 0.05 to 1.0 mg/kg, and most preferably about 0.2 mg/kg, and the therapeutic combination regimen described above is optionally followed by a maintenance dose of up to about 5 mg/kg PF3512676, preferably about 0.01 to 2.5 mg/kg, more preferably, about 0.05 to 1.0 mg/kg, and most preferably about 0.2 mg/kg. In a particularly preferred embodiment, the CpG ODN is PF-3512676. One skilled in the art can readily determine the optimal dosage for a particular patient based on tumor response and adverse event profile.

(b) Pemetrexed (ALIMTA®), pemetrexed for injection, which is supplied by Eli Lilli & Co., Indianapolis, Ind., is an antifolate antineoplastic agent that exerts its action by disrupting folate-dependent metabolic processes essential for cell replication. Pemetrexed disodium heptahydrate has the chemical name L-Glutamic acid, N-[4-[2-(2-amino-4,7-dihydro-4-oxo-1H-pyrrolo[2,3-d]pyrimidin-5-yl)ethyl]benzoyl]-, disodium salt, heptahydrate. Pemetrexed is an antifolate containing the pyrrolopyrimidine-based nucleus that exerts its antineoplastic activity by disrupting folate-dependent metabolic processes essential for cell replication.

For the treatment of a patient suffering from cancer as described herein, the recommended dose of Pemetrexed is up to about 1000 mg/m², preferably up to about 750 mg/m², most preferably up to about 500 mg/m², in combination with up to about 5 mg/m² CpG ODN, preferably PF3512676, 1018 ISS, Genasense, and IMOxine®, administered subcutaneously. In a preferred embodiment, PF3512676 is administered at a therapeutic dose of about 0.01 to 2.5 mg/kg, more preferably, about 0.05 to 1.0 mg/kg, and most preferably about 0.2 mg/kg, and the therapeutic combination regimen described above is optionally followed by a maintenance dose of up to about 5 mg/kg PF3512676, preferably about 0.01 to 2.5 mg/kg, more preferably, about 0.05 to 1.0 mg/kg, and most preferably about 0.2 mg/kg.

A premedication regimen may be used in order to mitigate certain side effects commonly associated with pemetrexed treatment. For example, skin rash has been reported more frequently in patients not pretreated with a corticosteroid. Therefore, pretreatment with dexamethasone (or equivalent) reduces the incidence and severity of cutaneous reaction. In clinical trials, dexamethasone 4 mg was given by mouth twice daily the day before, the day of, and the day after pemetrexed administration. Moreover, in order to reduce toxicity, patients treated with pemetrexed must be instructed to take a low-dose oral folic acid preparation or multivitamin with folic acid on a daily basis. At least 5 daily doses of folic acid may be taken during the 7-day period preceding the first dose of pemetrexed; and dosing may continue during the full course of therapy and for 21 days after the last dose of pemetrexed. Patients may also receive one (1) intramuscular injection of vitamin B12 during the week preceding the first dose of pemetrexed and every 3 cycles thereafter. Subsequent vitamin B12 injections may be given the same day as pemetrexed. In clinical trials, the dose of folic acid studied ranged from 350 to 1000 μg, and the dose of vitamin B12 was 1000 μg. The most commonly used dose of oral folic acid in clinical trials was 400 μg.

(c) Cisplatin (PLATINOL®-AQ) is supplied by Bristol Myers Squibb, Princeton, N.Y. The active ingredient, cisplatin, is a yellow to orange crystalline powder with the molecular formula PtCl₂H₆N₂, and a molecular weight of 300.1. Cisplatin is a heavy metal complex containing a central atom of platinum surrounded by two chloride atoms and two ammonia molecules in the cis position.

When used in combination with a CpG ODN, up to about 5 mg/kg CpG ODN, preferably PF3512676, 1018 ISS, Genasense, and IMOxine®, and most preferably PF-3512676, is administered at up to about 120 mg/m², preferably up to about 100 mg/m² cisplatin. As a non-limiting example of the use of this combination, for the treatment of first line NSCLC, cisplatin is administered in a treatment regimen in combination with up to about 5 mg/kg CpG ODN, preferably PF3512676, 1018 ISS, Genasense, and IMOxine®, most preferably PF-3512676, and paclitaxel, docetaxel, gemcitabine and/or vinorelbine. In such a treatment regimen, the treatment dosages are: up to about 100 mg/m², e.g., 50-100 mg/m², preferably 75 mg/m², cisplatin administered intravenously, in combination with up to about 5 mg/kg, preferably about 0.01-5.0 mg/kg CpG ODN, and optionally one or more of the following agents. (a) up to about 250 mg/m² paclitaxel, preferably up to about 225 mg/m², (b) up to about 100 mg/m², preferably up to about 75 mg/m² docetaxel, (c) up to about 1500 mg/m² gemcitabine, preferably 1250 mg/m², and/or (d) up to about 50 mg/m², and preferably up to about 30 mg/m² vinorelbine. The administration of cisplatin with one or more of the aforementioned agents may be done in a weekly treatment cycle, e.g., a 3-4 week treatment cycle, in any order, e.g., CpG ODN, followed by chemotherapeutics or vice versa, and optionally including a maintenance phase including a maintenance dose of CpG ODN following therapeutic regimen. In a preferred embodiment, PF3512676 is administered at a therapeutic dose of about 0.01 to 2.5 mg/kg, more preferably, about 0.05 to 1.0 mg/kg, and most preferably about 0.2 mg/kg, and the therapeutic combination regimen described above is optionally followed by a maintenance dose of up to about 5 mg/kg PF3512676, preferably about 0.01 to 2.5 mg/kg, more preferably, about 0.05 to 1.0 mg/kg, and most preferably about 0.2 mg/kg.

Pretreatment hydration with 1 to 2 liters of fluid infused for 8 to 12 hours prior to a cisplatin dose is recommended. The drug is then diluted in 2 liters of 5% Dextrose in ½ or ⅓ normal saline containing 37.5 g of mannitol, and infused over a 6- to 8-hour period. Adequate hydration and urinary output must be maintained during the following 24 hours. A repeat course of cisplatin should not be given until the serum creatinine is below 1.5 mg/100 mL, and/or the BUN is below 25 mg/100 mL. A repeat course should not be given until circulating blood elements are at an acceptable level (platelets ≧100,000/mm3, WBC ≧4,000/mm3). Subsequent doses of cisplatin should not be given until an audiometric analysis indicates that auditory acuity is within normal limits.

(d) Gemcitabine (Gemzar® HCl), which is supplied by Eli Lilly & Co., Indianapolis, Ind., is a nucleoside analogue that exhibits antitumor activity. Gemcitabine is 2′-deoxy-2′,2′-difluorocytidine monohydrochloride (β-isomer). Gemcitabine exhibits cell phase specificity, primarily killing cells undergoing DNA synthesis (S-phase) and also blocking the progression of cells through the G1/S-phase boundary. Gemcitabine is metabolized intracellularly by nucleoside kinases to the active diphosphate (dFdCDP) and triphosphate (dFdCTP) nucleosides. The cytotoxic effect of gemcitabine is attributed to a combination of two actions of the diphosphate and the triphosphate nucleosides, which leads to inhibition of DNA synthesis. First, gemcitabine diphosphate inhibits ribonucleotide reductase, which is responsible for catalyzing the reactions that generate the deoxynucleoside triphosphates for DNA synthesis. Inhibition of this enzyme by the diphosphate nucleoside causes a reduction in the concentrations of deoxynucleotides, including dCTP. Second, gemcitabine triphosphate competes with dCTP for incorporation into DNA. The reduction in the intracellular concentration of dCTP (by the action of the diphosphate) enhances the incorporation of gemcitabine triphosphate into DNA (self-potentiation). After the gemcitabine nucleotide is incorporated into DNA, only one additional nucleotide is added to the growing DNA strands. After this addition, there is inhibition of further DNA synthesis. DNA polymerase epsilon is unable to remove the gemcitabine nucleotide and repair the growing DNA strands (masked chain termination). In CEM T lymphoblastoid cells, gemcitabine induces internucleosomal DNA fragmentation, one of the characteristics of programmed cell death.

Gemcitabine demonstrated dose-dependent synergistic activity with cisplatin in vitro. No effect of cisplatin on gemcitabine triphosphate accumulation or DNA double-strand breaks was observed. In vivo, gemcitabine showed activity in combination with cisplatin against the LX-1 and CALU-6 human lung xenografts, but minimal activity was seen with the NCI-H460 or NCI-H520 xenografts. Gemcitabine was synergistic with cisplatin in the Lewis lung murine xenograft. Sequential exposure to gemcitabine 4 hours before cisplatin produced the greatest interaction.

In a preferred embodiment, gemcitabine is administered at a dose of up to about 1500 mg/m², preferably up to about 1250 mg/m², in combination with a CpG ODN. CpG ODN, preferably PF3512676, 1018 ISS, Genasense, and IMOxine®, most preferably PF-3512676, is administered subcutaneously in combination with gemcitabine at a dose of up to about 5 mg/kg CpG ODN. In a preferred embodiment, PF3512676 is administered at a therapeutic dose of about 0.01 to 2.5 mg/kg, more preferably, about 0.05 to 1.0 mg/kg, and most preferably about 0.2 mg/kg, and the therapeutic combination regimen described above is optionally followed by a maintenance dose of up to about 5 mg/kg PF3512676, preferably about 0.01 to 2.5 mg/kg, more preferably, about 0.05 to 1.0 mg/kg, and most preferably about 0.2 mg/kg.

In a non-limiting embodiment, for the treatment of NSCLC, gemcitabine may be administered in combination with cisplatin and a CpG ODN. This triplet therapy may be administered according to any of the therapeutic regimens described above with reference to the doublet regimen of gemcitabine and a CpG ODN. In a preferred embodiment, the gemcitabine, cisplatin and CpG ODN triplet regimen is administered according to the following non-limiting treatment regimen(s): in a 4-week schedule, Gemcitabine is administered intravenously at up to about 1250 mg/m² over about 1 hour, preferably about 30 minutes several times throughout a treatment regimen, e.g., once each week of a 3-4 week regimen, preferably on Days 1, 8, and 15 of each 28-day cycle. Cisplatin should be administered intravenously at up to about 100 mg/m² after the infusion of Gemcitabine, preferably on Day 1 after the administration of gemcitabine. CpG ODN, preferably PF3512676, 1018 ISS, Genasense, and IMOxine®, most preferably PF-3512676, is administered subcutaneously in combination with Gemcitabine and cisplatin at a dose of up to about 5 mg/kg CpG ODN. In a preferred embodiment, PF3512676 is administered at a therapeutic dose of about 0.01 to 2.5 mg/kg, more preferably, about 0.05 to 1.0 mg/kg, and most preferably about 0.2 mg/kg, and the therapeutic combination regimen described above is optionally followed by a maintenance dose of up to about 5 mg/kg PF3512676, preferably about 0.01 to 2.5 mg/kg, more preferably, about 0.05 to 1.0 mg/kg, and most preferably about 0.2 mg/kg. Alternatively, in a 3-week schedule, Gemcitabine should be administered intravenously at up to about 1250 mg/m² over up to about an hour, preferably up to about 30 minutes, several times throughout a treatment regimen, e.g., once a week of a 3-4 week regimen, preferably on Days 1 and 8 of each 21-day cycle. Cisplatin at a dose of up to about 100 mg/m² should be administered intravenously after the infusion of Gemcitabine, preferably on Day 1 after the infusion of Gemcitabine. CpG ODN, preferably PF3512676, 1018 ISS, genasense or IMOxine®, most preferably PFE-3512676, is administered subcutaneously in combination with Gemcitabine and cisplatin at a dose of up to about 5 mg/kg CpG ODN. In a preferred embodiment, PF3512676 is administered at a therapeutic dose of about 0.01 to 2.5 mg/kg, more preferably, about 0.05 to 1.0 mg/kg, and most preferably about 0.2 mg/kg, and the therapeutic combination regimen described above is optionally followed by a maintenance dose of up to about 5 mg/kg PF3512676, preferably about 0.01 to 2.5 mg/kg, more preferably, about 0.05 to 1.0 mg/kg, and most preferably about 0.2 mg/kg.

Dosage adjustments for hematologic toxicity may be required for gemcitabine and for cisplatin. Gemcitabine dosage adjustment for hematological toxicity is based on the granulocyte and platelet counts taken on the day of therapy. Patients receiving gemcitabine should be monitored prior to each dose with a complete blood count (CEC), including differential and platelet counts.

(e) Gefitinib (Iressa®), supplied by AstraZeneca, Wilmington, Del., is an anilinoquinazoline with the chemical name 4-Quinazolinamine,N-(3-chloro-4-fluorophenyl)-7-methoxy-6-[3-4-morpholin)propoxy]. The mechanism of the clinical antitumor action of gefitinib is not fully characterized. Gefitinib inhibits the intracellular phosphorylation of numerous tyrosine kinases associated with transmembrane cell surface receptors, including the tyrosine kinases associated with the epidermal growth factor receptor (EGFR-TK). EGFR is expressed on the cell surface of many normal cells and cancer cells. No clinical studies have been performed that demonstrate a correlation between EGFR receptor expression and response to gefitinib.

The recommended daily dose of Gefitinib is up to about 1000 mg, preferably up to 500 mg, most preferably up to about 250 mg, in combination with a up to about 5 mg/kg CpG ODN, preferably PF3512676, 1018 ISS, Genasense, or IMOxine®, preferably PF-3512676, administered subcutaneously. In a preferred embodiment, PF3512676 is administered at a therapeutic dose of about 0.01 to 2.5 mg/kg, more preferably, about 0.05 to 1.0 mg/kg, and most preferably about 0.2 mg/kg, and the therapeutic combination regimen described above is optionally followed by a maintenance dose of up to about 5 mg/kg PF3512676, preferably about 0.01 to 2.5 mg/kg, more preferably, about 0.05 to 1.0 mg/kg, and most preferably about 0.2 mg/kg.

(f) Paclitaxel (Taxol®) Injection is supplied by Bristol Myers Squibb, Princeton, N.J. Paclitaxel is a natural product with antitumor activity. Paclitaxel is obtained via a semi-synthetic process from Taxus baccata. The chemical name for paclitaxel is 5β,20-Epoxy-1,2α,4,7β,10β,13α-hexahydroxytax-11-en-9-one 4,10-diacetate 2-benzoate 13-ester with (2R,3S)—N-benzoyl-3-phenylisoserine. Paclitaxel is a novel antimicrotubule agent that promotes the assembly of microtubules from tubulin dimers and stabilizes microtubules by preventing depolymerization. This stability results in the inhibition of the normal dynamic reorganization of the microtubule network that is essential for vital interphase and mitotic cellular functions. In addition, paclitaxel induces abnormal arrays or “bundles” of microtubules throughout the cell cycle and multiple asters of microtubules during mitosis.

Paclitaxel may be administered in combination with up to about 5 mg/kg CpG ODN, such as PF3512676, 1018 ISS, Genasense, or IMOxine®, in combination with up to about 250 mg/m², preferably up to about 225 mg/m² paclitaxel. In a preferred embodiment, PF3512676 is administered at a therapeutic dose of about 0.01 to 2.5 mg/kg, more preferably, about 0.05 to 1.0 mg/kg, and most preferably about 0.2 mg/kg, and the therapeutic combination regimen described above is optionally followed by a maintenance dose of up to about 5 mg/kg PF3512676, preferably about 0.01 to 2.5 mg/kg, more preferably, about 0.05 to 1.0 mg/kg, and most preferably about 0.2 mg/kg.

For patients with NSCLC, the a non-limiting exemplary treatment regimen given every 3 weeks, is Paclitaxel administered intravenously over 24 hours at a dose of up to about 135 mg/m², followed by cisplatin, up to about 100 mg/m², in combination with up to about 5 mg/kg CpG ODN before, simultaneously, substantially simultaneously, or after administration of Paclitaxel, and the CpG ODN is preferably PF-3512676, 1018 ISS, Genasense, or IMOxine®, administered subcutaneously. In a preferred embodiment, PF3512676 is administered at a therapeutic dose of about 0.01 to 2.5 mg/kg, more preferably, about 0.05 to 1.0 mg/kg, and most preferably about 0.2 mg/kg, and the therapeutic combination regimen described above is optionally followed by a maintenance dose of up to about 5 mg/kg PF3512676, preferably about 0.01 to 2.5 mg/kg, more preferably, about 0.05 to 1.0 mg/kg, and most preferably about 0.2 mg/kg.

(g) Bevacizumab (Avastin™), supplied by Genentech, Inc., South San Francisco, Calif., is a recombinant humanized monoclonal IgG1 antibody that binds to and inhibits the biologic activity of human vascular endothelial growth factor (VEGF) in in vitro and in vivo assay systems. Bevacizumab contains human framework regions and the complementarity-determining regions of a murine antibody that binds to VEGF (1). Bevacizumab is produced in a Chinese Hamster Ovary mammalian cell expression system in a nutrient medium containing the antibiotic gentamicin and has a molecular weight of approximately 149 kilodaltons. Bevacizumab binds VEGF and prevents the interaction of VEGF to its receptors (Flt-1 and KDR) on the surface of endothelial cells. The interaction of VEGF with its receptors leads to endothelial cell proliferation and new blood vessel formation in in vitro models of angiogenesis. Administration of bevacizumab to xenotransplant models of colon cancer in nude (athymic) mice caused reduction of microvascular growth and inhibition of metastatic disease progression.

The recommended dose of Bevacizumab is up to about 15 mg/kg in combination with up to about 5 mg/kg CpG ODN, preferably PF3512676, 1018 ISS, Genasense, or IMOxine®, administered subcutaneously. In a preferred embodiment, PF3512676 is administered at a therapeutic dose of about 0.01 to 2.5 mg/kg, more preferably, about 0.05 to 1.0 mg/kg, and most preferably about 0.2 mg/kg, and the therapeutic combination regimen described above is optionally followed by a maintenance dose of up to about 5 mg/kg PF3512676, preferably about 0.01 to 2.5 mg/kg, more preferably, about 0.05 to 1.0 mg/kg, and most preferably about 0.2 mg/kg.

(h) Carboplatin (Paraplatin® aqueous solution) Injection is supplied by Bristol Myers Squibb, Princeton, N.J. Carboplatin is a platinum coordination compound. The chemical name for carboplatin is platinum, diammine [1,1-cyclobutane-dicarboxylato (2-)-0,0′]-,(SP-4-2). Carboplatin is a crystalline powder with the molecular formula of C₆H₁₂N₂O₄Pt and a molecular weight of 371.25. Carboplatin, like cisplatin, produces predominantly interstrand DNA crosslinks rather than DNA-protein cross-links. This effect is apparently cell-cycle nonspecific. The aquation of carboplatin, which is thought to produce the active species, occurs at a slower rate than in the case of cisplatin. Despite this difference, it appears that both carboplatin and cisplatin induce equal numbers of drug-DNA cross-links, causing equivalent lesions and biological effects. The differences in potencies for carboplatin and cisplatin appear to be directly related to the difference in aquation rates.

A simple formula for calculating dosage of carboplatin based upon a patient's glomerular filtration rate (GFR in mL/min) and carboplatin target area under the concentration versus time curve (AUC in mg/mL-min), has been proposed by Calvert. In these studies, GFR was measured by ⁵¹Cr-EDTA clearance, but calculated creatinine clearance is also commonly used in clinical practice. According to the Calvert formula, the total dose (mg) of carboplatin=(target AUC)×(GFR+25). Target AUC may be up to about 8, preferably up to about 6. This dose of carboplatin may be used in combination with up to about 5 mg/kg CpG ODN, preferably PF3512676, 1018 ISS, Genasense, or IMOxine®, administered subcutaneously. In a preferred embodiment, PF3512676 is administered at a therapeutic dose of about 0.01 to 2.5 mg/kg, more preferably, about 0.05 to 1.0 mg/kg, and most preferably about 0.2 mg/kg, and the therapeutic combination regimen described above is optionally followed by a maintenance dose of up to about 5 mg/kg PF3512676, preferably about 0.01 to 2.5 mg/kg, more preferably, about 0.06 to 1.0 mg/kg, and most preferably about 0.2 mg/kg.

In a non-limiting example, Carboplatin® (carboplatin aqueous solution) Injection, is administered at a dosage of as determined by the Calvert formula (see above), at the beginning of a treatment regimen, preferably within the first week of a treatment regimen, and most preferably on day 1 every 4 weeks, in combination with Paclitaxel and up to about 5 mg/kg CpG ODN, preferably PF-3512676, 1018 ISS, Genasense, or IMOxine®, administered subcutaneously. In a preferred embodiment, PF3512676 is administered at a therapeutic dose of about 0.01 to 2.5 mg/kg, more preferably, about 0.05 to 1.0 mg/kg, and most preferably about 0.2 mg/kg, and the therapeutic combination regimen described above is optionally followed by a maintenance dose of up to about 5 mg/kg PF3512676, preferably about 0.01 to 2.5 mg/kg, more preferably, about 0.05 to 1.0 mg/kg, and most preferably about 0.2 mg/kg.

(i) Erlotinib (Tarceva), supplied by OSI Pharmaceuticals, Melville, N.Y., is a Human Epidermal Growth Factor Receptor Type 1/Epidermal Growth Factor Receptor (HER1/EGFR) tyrosine kinase inhibitor. Erlotinib is a quinazolinamine with the chemical name N-(3-ethynylphenyl)-6,7-bis(2-methoxyethoxy)-4-quinazolinamine. Tarceva contains erlotinib as the hydrochloride salt. The mechanism of clinical antitumor action of erlotinib is not fully characterized. Erlotinib inhibits the intracellular phosphorylation of tyrosine kinase associated with the epidermal growth factor receptor (EGFR). Specificity of inhibition with regard to other tyrosine kinase receptors has not been fully characterized. EGFR is expressed on the cell surface of normal cells and cancer cells.

The recommended daily dose of erlotinib is up to about 200 mg, preferably up to 150 mg, in combination with up to about 5 mg/kg CpG ODN, preferably PF3512676, 1018 ISS, Genasense, or IMOxine®, administered subcutaneously. In a preferred embodiment, PF3512676 is administered at a therapeutic dose of about 0.01 to 2.5 mg/kg, more preferably, about 0.05 to 1.0 mg/kg, and most preferably about 0.2 mg/kg, and the therapeutic combination regimen described above is optionally followed by a maintenance dose of up to about 5 mg/kg PF3512676, preferably about 0.01 to 2.5 mg/kg, more preferably, about 0.05 to 1.0 mg/kg, and most preferably about 0.2 mg/kg.

(j) Imatinib (Gleevec™), a compound available from Novartis, East Hanover, N.J., as a tablet containing imatinib mesylate in 100 or 400 mg free base equivalent. Imatinib mesylate is designated chemically as 4-[(4-methyl-1-piperazinyl)methyl]-N-[4-methyl-3-[[4-(3-pyridinyl)-2-pyrimidinyl]amino]-phenyl]benzamide)methanesulfonate. Imatinib mesylate is a protein tyrosine kinase inhibitor that inhibits the bcr-abl tyrosine kinase, the constitutive abnormal tyrosine kinase created by the Philadelphia chromosome abnormality in chronic myeloid leukemia (CML). It inhibits proliferation and induces apoptosis in bcr-abl positive cell lines as well as fresh leukemic cells from Philadelphia chromosome positive chronic myeloid leukemia. In colony formation assays using ex vivo peripheral blood and bone marrow samples, imatinib shows inhibition of bcr-abl positive colonies from CML patients. In vivo, it inhibitors tumor growth of bcr-abl transfected murine myeloid cells as well as bcr-abl positive leukemia lines derived from CML patients in blast crisis. Imatinib is also an inhibitor of the receptor tyrosine kinases for platelet-derived growth factor (PDGF) and stem cell factor (SCF), c-kit, and inhibits PDGR- and SCF-mediated cellular events. In vitro, imatinib inhibits proliferation and induces apoptosis in gastrointestinal stromal tumor (GIST) cells, which express an activating c-kit mutation.

The recommended dosage of imatinib is up to about up to about 1000 mg, preferably up to about 800 mg, e.g., 600 mg/day, e.g., 400 mg/day or 600 mg/day, in combination with up to about 5 mg/kg CpG ODN, preferably PF3512676, 1018 ISS, Genasense, or IMOxine®, administered subcutaneously. In a preferred embodiment, PF3512676 is administered at a therapeutic dose of about 0.01 to 2.5 mg/kg, more preferably, about 0.05 to 1.0 mg/kg, and most preferably about 0.2 mg/kg, and the therapeutic combination regimen described above is optionally followed by a maintenance dose of up to about 5 mg/kg PF3512676, preferably about 0.01 to 2.5 mg/kg, more preferably, about 0.05 to 1.0 mg/kg, and most preferably about 0.2 mg/kg.

(k) Sorafenib (BAY 43-9006; N-[4-Chloro-3-(trifluoromethyl)phenyl]-N′-[4-[2-(N-methylcarbamoyl)-4-pyridyloxy]phenyl]urea), a novel investigational drug candidate from Onyx Pharmaceuticals, Emeryville, Calif., and Bayer Pharmaceuticals, West Haven, Conn., is the first oral multi-kinase inhibitor that targets serine/threonine and receptor tyrosine kinases in both the tumor cell and tumor vasculature. In preclinical models, sorafenib targeted members of two classes of kinases known to be involved in both tumor cell proliferation (tumor growth) and tumor angiogenesis (tumor blood supply)—two important cancer growth activities. These kinases included RAF kinase, VEGFR-2, VEGFR-3, PDGFR-β, KIT, FLT-3 and RET.

The recommended dose of Sorafenib is up to about 1500 mg, preferably up to about 1000 mg, and preferably 400 mg twice daily, in combination with up to about 5 mg/kg CpG ODN, preferably PF-3512676, 1018 ISS, Genasense, or IMOxine®, administered subcutaneously. In a preferred embodiment, PF3512676 is administered at a therapeutic dose of about 0.01 to 2.5 mg/kg, more preferably, about 0.05 to 1.0 mg/kg, and most preferably about 0.2 mg/kg, and the therapeutic combination regimen described above is optionally followed by a maintenance dose of up to about 5 mg/kg PF3512676, preferably about 0.01 to 2.5 mg/kg, more preferably, about 0.05 to 1.0 mg/kg, and most preferably about 0.2 mg/kg.

(l) Docetaxel (Taxotere®) is supplied by Sanofi-Aventis. Docetaxel is an antineoplastic agent belonging to the taxoid family. It is prepared by semisynthesis beginning with a precursor extracted from the renewable needle biomass of yew plants. The chemical name for docetaxel is (2R,3S)—N-carboxy-3-phenylisoserine,N-tert-butyl ester, 13-ester with 5β-20-epoxy-1,2α,4,7β,10β,13α-hexahydroxytax-11-en-9-one 4-acetate 2-benzoate, trihydrate. Docetaxel is an antineoplastic agent that acts by disrupting the microtubular network in cells that is essential for mitotic and interphase cellular functions. Docetaxel binds to free tubulin and promotes the assembly of tubulin into stable microtubules while simultaneously inhibiting their disassembly. This leads to the production of microtubule bundles without normal function and to the stabilization of microtubules, which results in the inhibition of mitosis in cells. Docetaxel's binding to microtubules does not alter the number of protofilaments in the bound microtubules, a feature which differs from most spindle poisons currently in clinical use.

For the treatment of breast cancer, the recommended dose of docetaxel is up to about 120 mg/m², preferably up to about 100 mg/m², e.g., 60-100 mg/m², in combination with up to about 5 mg/kg CpG ODN, preferably PF-3512676, 1018 ISS, Genasense, or IMOxine®, administered subcutaneously. This dose may be modified according to the treatment setting, e.g., in the adjuvant treatment of operable node-positive breast cancer, the recommended docetaxel dose is up to about 75 mg/m² administered 1-hour after doxorubicin, up to about 50 mg/m², and cyclophosphamide, up to about 500 mg/m² every 3 weeks for 6 courses, in combination with up to about 5 mg/kg CpG ODN, preferably PF-3512676, 1018 ISS, Genasense, or IMOxine®, administered subcutaneously. Prophylactic G-CSF may be used to mitigate the risk of hematological toxicities. In a preferred embodiment, PF3512676 is administered at a therapeutic dose of about 0.01 to 2.5 mg/kg, more preferably, about 0.05 to 1.0 mg/kg, and most preferably about 0.2 mg/kg, and the therapeutic combination regimen described above is optionally followed by a maintenance dose of up to about 5 mg/kg PF3512676, preferably about 0.01 to 2.5 mg/kg, more preferably, about 0.05 to 1.0 mg/kg, and most preferably about 0.2 mg/kg.

For example, in the treatment of NSCLC the recommended dose of docetaxel is up to about 100 mg/m2, preferably up to about 75 mg/m² administered intravenously over 1 hour every 3 weeks, in combination with up to about 5 mg/kg CpG ODN, preferably PF-3512676, 1018 ISS, Genasense, or IMOxine®, administered subcutaneously. For chemotherapy-naïve patients, the recommended dose of docetaxel is up to about 75 mg/m² administered intravenously over 1 hour immediately followed by cisplatin, up to about 100 mg/m2, preferably up to about 75 mg/m² over 30-60 minutes every 3 weeks, in combination with up to about 5 mg/kg CpG ODN, preferably PF-3512676, 1018 ISS, Genasense, or IMOxine®, administered subcutaneously. In a preferred embodiment, PF3512676 is administered at a therapeutic dose of about 0.01 to 2.5 mg/kg, more preferably, about 0.05 to 1.0 mg/kg, and most preferably about 0.2 mg/kg, and the therapeutic combination regimen described above is optionally followed by a maintenance dose of up to about 5 mg/kg PF3512676, preferably about 0.01 to 2.5 mg/kg, more preferably, about 0.05 to 1.0 mg/kg, and most preferably about 0.2 mg/kg.

(m) Vinorelbine tartrate (Navelbine; referred to hereinbelow as vinorelbine) is supplied by GlaxoSmithkline, Research Triangle Park, N.C. Vinorelbine tartrate is a semi-synthetic vinca alkaloid with antitumor activity. The chemical name is 3′,4′-didehydro-4′-deoxy-C′-norvincaleukoblastine[R—(R*,R*)-2,3-dihydroxybutanedioate (1:2) (salt)]. Vinorelbine is a vinca alkaloid that interferes with microtubule assembly. The vinca alkaloids are structurally similar compounds comprised of 2 multiringed units, vindoline and catharanthine. Unlike other vinca alkaloids, the catharanthine unit is the site of structural modification for vinorelbine. The antitumor activity of vinorelbine is thought to be due primarily to inhibition of mitosis at metaphase through its interaction with tubulin. Like other vinca alkaloids, vinorelbine may also interfere with: 1) amino acid, cyclic AMP, and glutathione metabolism, 2) calmodulin-dependent Ca++-transport ATPase activity, 3) cellular respiration, and 4) nucleic acid and lipid biosynthesis. In intact tectal plates from mouse embryos, vinorelbine, vincristine, and vinblastine inhibited mitotic microtubule formation at the same concentration (2 μM), inducing a blockade of cells at metaphase. Vincristine produced depolymerization of axonal microtubules at 5 μM, but vinblastine and vinorelbine did not have this effect until concentrations of 30 μM and 40 μM, respectively. These data suggest relative selectivity of vinorelbine for mitotic microtubules.

The usual initial dose of single-agent vinorelbine is up to about 50 mg/m², preferably up to about 30 mg/m² administered weekly, in combination with up to about 5 mg/kg CpG ODN, preferably PF-3512676, 1018 ISS, Genasense, or IMOxine®, administered subcutaneously. Vinorelbine may be administered by any suitable method in a single bolus or over a defined period of time. For example, vinorelbine is administered as an intravenous injection over a period of time, e.g., up to 1 hour, preferably up to 30 minutes, most preferably up to 10 minutes. In controlled trials, single-agent vinorelbine was given weekly until progression or dose-limiting toxicity. Alternatively, vinorelbine may be administered weekly at a dose of up to about 25 mg/m² in combination with cisplatin given every 4 weeks at a dose of up to about 100 mg/m², in combination with up to about 5 mg/kg CpG ODN, preferably PF-3512676, 1018 ISS, or IMOxine®, administered subcutaneously. In a preferred embodiment, PF3512676 is administered at a therapeutic dose of about 0.01 to 2.5 mg/kg, more preferably, about 0.05 to 1.0 mg/kg, and most preferably about 0.2 mg/kg, and the therapeutic combination regimen described above is optionally followed by a maintenance dose of up to about 5 mg/kg PF3512676, preferably about 0.01 to 2.5 mg/kg, more preferably, about 0.05 to 1.0 mg/kg, and most preferably about 0.2 mg/kg.

(n) Irinotecan (Camptosar®; Irinotecan hydrochloride) is supplied by Pfizer Inc., New York, N.Y. Irinotecan hydrochloride injection is an antineoplastic agent of the topoisomerase I inhibitor class. Irinotecan hydrochloride was clinically investigated as CPT-11. Irinotecan hydrochloride is a semisynthetic derivative of camptothecin, an alkaloid extract from plants such as Camptotheca acuminata. The chemical name is (S)-4,11-diethyl-3,4,12,14-tetrahydro-4-hydroxy-3,14-dioxol H-pyrano[3′,4′:6,7]-indolizino[1,2-b]quinolin-9-yl-[1,4′bipiperidine]-1′-carboxylate, monohydrochloride, trihydrate. Irinotecan is a derivative of camptothecin. Camptothecins interact specifically with the enzyme topoisomerase I which relieves torsional strain in DNA by inducing reversible single-strand breaks. Irinotecan and its active metabolite SN-38 bind to the topoisomerase I-DNA complex and prevent religation of these single-strand breaks. Current research suggests that the cytotoxicity of irinotecan is due to double-strand DNA damage produced during DNA synthesis when replication enzymes interact with the ternary complex formed by topoisomerase I, DNA, and either irinotecan or SN-38. Mammalian cells cannot efficiently repair these double-strand breaks. Irinotecan serves as a water-soluble precursor of the lipophilic metabolite SN-38. SN-38 is formed from irinotecan by carboxylesterase-mediated cleavage of the carbamate bond between the camptothecin moiety and the dipiperidino side chain. SN-38 is approximately 1000 times as potent as irinotecan as an inhibitor of topoisomerase I purified from human and rodent tumor cell lines. In vitro cytotoxicity assays show that the potency of SN-38 relative to irinotecan varies from 2- to 2000-fold. However, the plasma area under the concentration versus time curve (AUC) values for SN-38 are 2% to 8% of irinotecan and SN-38 is 95% bound to plasma proteins compared to approximately 50% bound to plasma proteins for irinotecan (see Pharmacokinetics). The precise contribution of SN-38 to the activity of Irinotecan® is thus unknown. Both irinotecan and SN-38 exist in an active lactone form and an inactive hydroxy acid anion form. A pH-dependent equilibrium exists between the two forms such that an acid pH promotes the formation of the lactone, while a more basic pH favors the hydroxy acid anion form.

Irinotecan is administered to patients at a dose of up to about 500 mg/m2, preferably up to about 375 mg/m², in combination with up to about 5 mg/kg CpG ODN, preferably PF-3512676, 1018 ISS, Genasense, or IMOxine®, administered subcutaneously. In a preferred embodiment, PF3512676 is administered at a therapeutic dose of about 0.01 to 2.5 mg/kg, more preferably, about 0.05 to 1.0 mg/kg, and most preferably about 0.2 mg/kg, and the therapeutic combination regimen described above is optionally followed by a maintenance dose of up to about 5 mg/kg PF3512676, preferably about 0.01 to 2.5 mg/kg, more preferably, about 0.05 to 1.0 mg/kg, and most preferably about 0.2 mg/kg.

(o) Etoposide (also commonly known as VP-16) is a semisynthetic derivative of podophyllotoxin used in the treatment of certain neoplastic diseases. It is 4′-Demethylepipodophyllotoxin 9-[4,6-0-(R)-ethylidene-β-D-glucopyranoside]. Etoposide is supplied by Gensia Sicor Pharmaceuticals, Inc., Irvine, Calif. Etoposide has been shown to cause metaphase arrest in chick fibroblasts. Its main effect, however, appears to be at the G2 portion of the cell cycle in mammalian cells. Two different dose-dependent responses are seen. At high concentrations (10 mcg/mL or more), lysis of cells entering mitosis is observed. At low concentrations (0.3 to 10 mcg/mL), cells are inhibited from entering prophase. It does not interfere with microtubular assembly. The predominant macromolecular effect of etoposide appears to be DNA synthesis inhibition.

The Etoposide Injection dose is up to about 250 mg/m², preferably up to about 100 mg/m², in combination with up to about 5 mg/kg CpG ODN, preferably PF-3512676, 1018 ISS, Genasense, or IMOxine®, administered subcutaneously. In a preferred embodiment, PF3512676 is administered at a therapeutic dose of about 0.01 to 2.5 mg/kg, more preferably, about 0.05 to 1.0 mg/kg, and most preferably about 0.2 mg/kg, and the therapeutic combination regimen described above is optionally followed by a maintenance dose of up to about 5 mg/kg PF3512676, preferably about 0.01 to 2.5 mg/kg, more preferably, about 0.05 to 1.0 mg/kg, and most preferably about 0.2 mg/kg.

(p) Vinblastine, Vincaleukoblastine, VLB, Velban (Sulfate) is supplied by Eton Biomedical, Inc., San Diego, Calif. Vinblastine is an alkaloid found in the Madagascar periwinkle, Catharanthus roseus. It binds to tubulin, preventing the cell from making the spindles it needs to be able to move its chromosomes around as it divides in metaphase of mitosis. It also seems to interfere with cell's ability to synthesize DNA and RNA.

Vinblastine is administered at a dose of up to about 10 mg/m², preferably up to about 5 mg/m², most preferably up to about 3 mg/m², in combination with up to about 5 mg/kg CpG ODN, preferably PF-3512676, 1018 ISS, Genasense, or IMOxine®, administered subcutaneously. In a preferred embodiment, PF3512676 is administered at a therapeutic dose of about 0.01 to 2.5 mg/kg, more preferably, about 0.05 to 1.0 mg/kg, and most preferably about 0.2 mg/kg, and the therapeutic combination regimen described above is optionally followed by a maintenance dose of up to about 5 mg/kg PF3512676, preferably about 0.01 to 2.5 mg/kg, more preferably, about 0.05 to 1.0 mg/kg, and most preferably about 0.2 mg/kg.

(q) Ifosfamide (IFEX®, Ifosfamide for injection) is supplied by Bristol-Myers Squibb, Princeton, N.J. Ifosfamide is a chemotherapeutic agent chemically related to the nitrogen mustards and a synthetic analog of cyclophosphamide. Ifosfamide is 3-(2-chloroethyl)-2-[(2-chloroethyl)amino]tetrahydro-2H-1,3,2-oxazaphosphorine 2-oxide. Ifosfamide has been shown to require metabolic activation by microsomal liver enzymes to produce biologically active metabolites. Activation occurs by hydroxylation at the ring carbon atom 4 to form the unstable intermediate 4-hydroxyifosfamide. This metabolite rapidly degrades to the stable urinary metabolite 4-ketoifosfamide. Opening of the ring results in formation of the stable urinary metabolite, 4-carboxylfosfamide. These urinary metabolites have not been found to be cytotoxic. N,N-bis(2-chloroethyl)-phosphoric acid diamide (ifosphoramide) and acrolein are also found. Enzymatic oxidation of the chloroethyl side chains and subsequent dealkylation produces the major urinary metabolites, dechloroethyl ifosfamide and dechloroethyl cyclophosphamide. The alkylated metabolites of ifosfamide have been shown to interact with DNA. In vitro incubation of DNA with activated ifosfamide has produced phosphotriesters. The treatment of intact cell nuclei may also result in the formation of DNA-DNA cross-links. DNA repair most likely occurs in G-1 and G-2 stage cells.

IFEX should be administered intravenously at a dose of up to about 3 g/m², preferably up to about 1.2 g/m², in combination with up to about CpG ODN, preferably PF-3512676, 1018 ISS, Genasense, or IMOxine®, administered subcutaneously. In a non-limiting example, IFEX is administered each day in a treatment regimen, e.g., for 3-7 and preferably 5 consecutive days, and treatment is optionally repeated every 3 weeks or after recovery from hematologic toxicity (Platelets ≧100,000/μL, WBC ≧4,000/μL). In a preferred embodiment, PF3512676 is administered at a therapeutic dose of about 0.01 to 2.5 mg/kg, more preferably, about 0.05 to 1.0 mg/kg, and most preferably about 0.2 mg/kg, and the therapeutic combination regimen described above is optionally followed by a maintenance dose of up to about 5 mg/kg PF3512676, preferably about 0.01 to 2.5 mg/kg, more preferably, about 0.05 to 1.0 mg/kg, and most preferably about 0.2 mg/kg.

(r) Topotecan (Hycamtin®; Topotecan hydrochloride) is supplied by GlaxosmithKline, Research Triangle Park, N.C. Topotecan is a semi-synthetic analogue of camptothecin, an agent derived from the Oriental yew tree, Camptothecan accuminata. The cytotoxic effects of the camptothecins are believed to be related to their activity as inhibitors of topoisomerase-I, an enzyme involved in the replication and repair of nuclear DNA. As DNA is replicated in dividing cells, topoisomerase-I acts by binding to super-coiled DNA and causing single-stranded breaks in that DNA. As a result, topoisomerase-I is able to relieve the torsional stresses that are introduced into DNA ahead of the replication complex or moving replication fork. Topotecan inhibits topoisomerase-I by stabilizing the covalent complex of enzyme and strand-cleaved DNA, which is an intermediate of the catalytic mechanism, thereby inducing breaks in the protein-associated DNA single-strands, resulting in cell death.

The recommended dose of Topotecan is up to about 5 mg/m2, preferably up to about 3 mg/m², most preferably up to about 1.5 mg/m², in combination with up to about 5 mg/kg CpG ODN, preferably PF-3512676, 1018 ISS, Genasense, or IMOxine®, administered subcutaneously. Prior to administration of the first course of topotecan, patients should have a baseline neutrophil count of >1,500 cells/mm³ and a platelet count of >100,000 cells/mm³; and the dose of topotecan is administered by intravenous infusion over a defined period of time, e.g., up to 3 hours, preferably up to 1 hour, most preferably up to about 30 minutes, daily for up to about five consecutive days, starting at the beginning, e.g., on day one, of a treatment regimen, e.g., a 21-day course In a preferred embodiment, PF3512676 is administered at a therapeutic dose of about 0.01 to 2.5 mg/kg, more preferably, about 0.05 to 1.0 mg/kg, and most preferably about 0.2 mg/kg, and the therapeutic combination regimen described above is optionally followed by a maintenance dose of up to about 5 mg/kg PF3512676, preferably about 0.01 to 2.5 mg/kg, more preferably, about 0.05 to 1.0 mg/kg, and most preferably about 0.2 mg/kg.

(s) Cyclophosphamide (Cytoxan®; Cyclophosphamide for injection of tablets) is supplied by Bristol Myers Squibb, Princeton, N.J. Cyclophosphamide is a synthetic antineoplastic drug chemically related to the nitrogen mustards. The chemical name for cyclophosphamide is 2-[bis(2-chloroethyl)amino]tetrahydro-2H-1,3,2-oxazaphosphorine 2-oxide monohydrate. Cyclophosphamide is biotransformed principally in the liver to active alkylating metabolites by a mixed function microsomal oxidase system. These metabolites interfere with the growth of susceptible rapidly proliferating malignant cells. The mechanism of action is thought to involve cross-linking of tumor cell DNA.

Cyclophosphamide is administered in intravenous regimens that include up to about 7 g/m², e.g., up to about 750 mg/m², given every 7 to 10 days, in combination with up to about 5 mg/kg CpG ODN, preferably PF-3512676, 1018 ISS, Genasense, or IMOxine®, administered subcutaneously. In a preferred embodiment, PF3512676 is administered at a therapeutic dose of about 0.01 to 2.5 mg/kg, more preferably, about 0.05 to 1.0 mg/kg, and most preferably about 0.2 mg/kg, and the therapeutic combination regimen described above is optionally followed by a maintenance dose of up to about 5 mg/kg PF3512676, preferably about 0.01 to 2.5 mg/kg, more preferably, about 0.05 to 1.0 mg/kg, and most preferably about 0.2 mg/kg.

(t) Vincristine (Oncovin®) is supplied by AM Pharm Partners. Vincristine belongs to the general group of chemotherapy drugs known as plant (vinca) alkaloids. Vincristine stops cell division, resulting in cell death.

Vincristine is given at a dose of up to about 30 mg/kg, e.g., 10-30 mg/kg or about 2 mg/m², in combination with up to about 5 mg/kg CpG ODN, preferably PF-3512676, 1018 ISS, Genasense, or IMOxine®, administered subcutaneously. In a preferred embodiment, PF3512676 is administered at a therapeutic dose of about 0.01 to 2.5 mg/kg, more preferably, about 0.05 to 1.0 mg/kg, and most preferably about 0.2 mg/kg, and the therapeutic combination regimen described above is optionally followed by a maintenance dose of up to about 5 mg/kg PF3512676, preferably about 0.01 to 2.5 mg/kg, more preferably, about 0.05 to 1.0 mg/kg, and most preferably about 0.2 mg/kg.

(u) Methotrexate (formerly Amethopterin) is an antimetabolite used in the treatment of certain neoplastic diseases, severe psoriasis, and adult rheumatoid arthritis. Methotrexate is supplied by Ben Venue Laboratories, Bedford, Ohio. Chemically methotrexate is N-[4-[[(2,4-diamino-6-pteridinyl)methyl]methylamino]benzoyl]-L-glutamic acid. Methotrexate inhibits dihydrofolic acid reductase. Dihydrofolates must be reduced to tetrahydrofolates by this enzyme before they can be utilized as carriers of one-carbon groups in the synthesis of p.r.n. nucleotides and thymidylate. Therefore, methotrexate interferes with DNA synthesis, repair, and cellular replication. Actively proliferating tissues such as malignant cells, bone marrow, fetal cells, buccal and intestinal mucosa, and cells of the urinary bladder are in general more sensitive to this effect of methotrexate. When cellular proliferation in malignant tissues is greater than in most normal tissues, methotrexate may impair malignant growth without irreversible damage to normal tissues.

Methotrexate is administered at doses of up to about 12 g/m², preferably up to about 50 mg/m², preferably up to about 30 mg/m², in combination with up to about 5 mg/kg CpG ODN, preferably PF-3512676, 1018 ISS, Genasense, or IMOxine®, administered subcutaneously. In a preferred embodiment, PF3512676 is administered at a therapeutic dose of about 0.01 to 2.5 mg/kg, more preferably, about 0.05 to 1.0 mg/kg, and most preferably about 0.2 mg/kg, and the therapeutic combination regimen described above is optionally followed by a maintenance dose of up to about 5 mg/kg PF3512676, preferably about 0.01 to 2.5 mg/kg, more preferably, about 0.05 to 1.0 mg/kg, and most preferably about 0.2 mg/kg.

(v) Fluorouracil (5-fluorouracil or 5-FU) is an antimetabolite that is available for systemic or topical administration.

Fluorouracil is administered at a dose of up to about 2600 mg/m², preferably up to about 500 mg/m², in combination with up to about 5 mg/kg CpG ODN, preferably PF-3512676, 1018 ISS, Genasense, or IMOxine®, administered subcutaneously. In a preferred embodiment, PF3512676 is administered at a therapeutic dose of about 0.01 to 2.5 mg/kg, more preferably, about 0.05 to 1.0 mg/kg, and most preferably about 0.2 mg/kg, and the therapeutic combination regimen described above is optionally followed by a maintenance dose of up to about 5 mg/kg PF3512676, preferably about 0.01 to 2.5 mg/kg, more preferably, about 0.05 to 1.0 mg/kg, and most preferably about 0.2 mg/kg.

(w) Capecitabine (Xeloda®) is supplied by Hoffman LaRoche, Nutley, N.J. Capecitabine is a fluoropyrimidine carbamate with antineoplastic activity. It is an orally administered systemic prodrug of 5′-deoxy-5-fluorouridine (5′-DFUR) which is converted to 5-fluorouracil. The chemical name for capecitabine is 5′-deoxy-5-fluoro-N-[(pentyloxy) carbonyl]-cytidine. Both normal and tumor cells metabolize 5-FU to 5-fluoro-2′-deoxyuridine monophosphate (FdUMP) and 5-fluorouridine triphosphate (FUTP). These metabolites cause cell injury by two different mechanisms. First, FdUMP and the folate cofactor, N⁵⁻¹⁰-methylenetetrahydrofolate, bind to thymidylate synthase (TS) to form a covalently bound ternary complex. This binding inhibits the formation of thymidylate from 2′-deoxyuridylate. Thymidylate is the necessary precursor of thymidine triphosphate, which is essential for the synthesis of DNA, so that a deficiency of this compound can inhibit cell division. Second, nuclear transcriptional enzymes can mistakenly incorporate FUTP in place of uridine triphosphate (UTP) during the synthesis of RNA. This metabolic error can interfere with RNA processing and protein synthesis.

The recommended dose of Capecitabine is up to about 2500 mg/m² preferably up to about 1250 mg/m² in combination with up to about 5 mg/kg CpG ODN, preferably PF-3512676, 1018 ISS, Genasense, or IMOxine®, administered subcutaneously. In a non-limiting example, capecitabine is administered orally twice daily (morning and evening; equivalent to up to about 2500 mg/m² total daily dose) for 2 weeks followed by a 1-week rest period given as 3-week cycles. In combination with docetaxel, the recommended dose of Capecitabine is up to about 1500 mg/m², preferably up to about 1250 mg/m² twice daily for 2 weeks followed by a 1-week rest period, combined with docetaxel at up to about 75 mg/m² as a 1-hour intravenous infusion every 3 weeks, in combination with up to about 5 mg/kg CpG ODN, preferably PF-3512676, 1018 ISS, or IMOxine®, administered subcutaneously. In a preferred embodiment, PF3512676 is administered at a therapeutic dose of about 0.01 to 2.5 mg/kg, more preferably, about 0.05 to 1.0 mg/kg, and most preferably about 0.2 mg/kg, and the therapeutic combination regimen described above is optionally followed by a maintenance dose of up to about 5 mg/kg PF3512676, preferably about 0.01 to 2.5 mg/kg, more preferably, about 0.05 to 1.0 mg/kg, and most preferably about 0.2 mg/kg.

(x) Doxorubicin (Adriamycin®; Hydroxyl daunorubicin) is supplied by Pharmacia & Upjohn. Daunorubicin and its 14-hydroxy derivative, doxorubicin, are anthracycline antibiotics produced by the fungus streptomyces peucetius. Doxorubicin damages DNA by intercalation of the anthracycline portion, metal ion chelation, or by generation of free radicals. Doxorubicin has also been shown to inhibit DNA topoisomerase II which is critical to DNA function. Cytotoxic activity is cell cycle phase-nonspecific.

Doxorubicin is administered up to about 75 mg/m², preferably up to about 50 mg/m², in combination with up to about 5 mg/kg CpG ODN, preferably PF-3512676, 1018 ISS, Genasense, or IMOxine®, administered subcutaneously. In a preferred embodiment, PF3512676 is administered at a therapeutic dose of about 0.01 to 2.5 mg/kg, more preferably, about 0.05 to 1.0 mg/kg, and most preferably about 0.2 mg/kg, and the therapeutic combination regimen described above is optionally followed by a maintenance dose of up to about 5 mg/kg PF3512676, preferably about 0.01 to 2.5 mg/kg, more preferably, about 0.05 to 1.0 mg/kg, and most preferably about 0.2 mg/kg.

(y) Epirubicin (Ellence®; Epirubicin hydrochloride injection) is supplied by Pharmacia & Upjohn. Epirubicin hydrochloride is the 4-epimer of doxbrubicin and is a semi-synthetic derivative of daunorubicin. The chemical name is (8S-cis)-10-[(3-amino-2,3,6-trideoxy-a-L-arabino-hexopyranosyl)oxy]-7,8,9,10-tetrahydro-6,8,11-trihydroxy-8-(hydroxyacetyl)-1-methoxy-5,12-naphthacenedione hydrochloride.

Epirubicin is administered at a dose of up to about 150 mg/m2, preferably up to about 135 mg/m2, or up to about 120 mg/m2, in combination with up to about 5 mg/kg CpG ODN, preferably PF-3512676, 1018 ISS, Genasense, or IMOxine®, administered subcutaneously. Epirubicin is optionally administered to patients by intravenous infusion in repeated 3- to 4-week cycles. The total dose of Epirubicin may be given on Day 1 of each cycle or divided equally and given on Days 1 and 8 of each cycle. In a preferred embodiment, PF3512676 is administered at a therapeutic dose of about 0.01 to 2.5 mg/kg, more preferably, about 0.05 to 1.0 mg/kg, and most preferably about 0.2 mg/kg, and the therapeutic combination regimen described above is optionally followed by a maintenance dose of up to about 5 mg/kg PF3512676, preferably about 0.01 to 2.5 mg/kg, more preferably, about 0.05 to 1.0 mg/kg, and most preferably about 0.2 mg/kg.

(z) Trastuzumab (Herceptin) is supplied by Genentech, Inc. Trastuzumab (Trastuzumab) is a recombinant DNA-derived humanized monoclonal antibody that selectively binds with high affinity in a cell-based assay (Kd=5 nM) to the extracellular domain of the human epidermal growth factor receptor 2 protein, HER2. The antibody is an IgG₁ kappa that contains human framework regions with the complementarity-determining regions of a murine antibody (4D5) that binds to HER2. The HER2 (or c-erbB2) protooncogene encodes a transmembrane receptor protein of 185 kDa, which is structurally related to the epidermal growth factor receptor. HER2 protein overexpression is observed in 25%-30% of primary breast cancers.

The recommended dose is up to about 8 mg/kg Trastuzumab, in combination with up to about 5 mg/kg CpG ODN, preferably PF-3512676, 1018 ISS, Genasense, or IMOxine®, administered subcutaneously. In a preferred embodiment, PF3512676 is administered at a therapeutic dose of about 0.01 to 2.5 mg/kg, more preferably, about 0.05 to 1.0 mg/kg, and most preferably about 0.2 mg/kg, and the therapeutic combination regimen described above is optionally followed by a maintenance dose of up to about 5 mg/kg PF3512676, preferably about 0.01 to 2.5 mg/kg, more preferably, about 0.05 to 1.0 mg/kg, and most preferably about 0.2 mg/kg.

(aa) Tamoxifen (Nolvadex®; tamoxifen citrate tablets) is supplied by AstraZeneca Pharmaceuticals, LP, Wilmington, Del. Tamoxifen is a nonsteroidal antiestrogen. The chemical name is (Z)2-[4-(1,2-diphenyl-1-butenyl)phenoxy]-N, Ndimethylethanamine 2-hydroxy-1,2,3-propanetricarboxylate (1:1). Nolvadex® is a nonsteroidal agent that has demonstrated potent antiestrogenic properties in animal test systems. The antiestrogenic effects may be related to its ability to compete with estrogen for binding sites in target tissues such as breast. Tamoxifen inhibits the induction of rat mammary carcinoma induced by dimethylbenzanthracene (DMBA) and causes the regression of already established DMBA-induced tumors. In this rat model, tamoxifen appears to exert its antitumor effects by binding the estrogen receptors. In cytosols derived from human breast adenocarcinomas, tamoxifen competes with estradiol for estrogen receptor protein.

For patients with breast cancer, the recommended daily dose is up to about 80 mg, e.g., 20-40 mg. Dosages greater than 20 mg per day should be given in divided doses (morning and evening). Tamoxifen may be administered in combination with up to about 5 mg/kg CpG ODN, preferably PF-3512676, 1018 ISS, Genasense, or IMOxine®, administered subcutaneously. In a preferred embodiment, PF3512676 is administered at a therapeutic dose of about 0.01 to 2.5 mg/kg, more preferably, about 0.05 to 1.0 mg/kg, and most preferably about 0.2 mg/kg, and the therapeutic combination regimen described above is optionally followed by a maintenance dose of up to about 5 mg/kg PF3512676, preferably about 0.01 to 2.5 mg/kg, more preferably, about 0.05 to 1.0 mg/kg, and most preferably about 0.2 mg/kg.

(bb) Letrozole (Femara®; Letrozole tablets) is supplied by Novartis, East Hanover, N.J. Letrozole is a nonsteroidal aromatase inhibitor, chemically described as 4,4′-(1H-1,2,4-Triazol-1-ylmethylene)dibenzonitrile.

Letrozole is administered up to 5 mg, without regard to meals, in combination with up to about 5 mg/kg CpG ODN, preferably PF-3512676, 1018 ISS, Genasense, or IMOxine®, administered subcutaneously. In a preferred embodiment, PF3512676 is administered at a therapeutic dose of about 0.01 to 2.5 mg/kg, more preferably, about 0.05 to 1.0 mg/kg, and most preferably about 0.2 mg/kg, and the therapeutic combination regimen described above is optionally followed by a maintenance dose of up to about 5 mg/kg PF3512676, preferably about 0.01 to 2.5 mg/kg, more preferably, about 0.05 to 1.0 mg/kg, and most preferably about 0.2 mg/kg.

(cc) Anastrozole (Arimidex®; Anastrozole tablets) is supplied by AstraZeneca Pharmaceuticals, LP, Wilmington, Del. Anastrozole is a non-steroidal aromatase inhibitor, chemically known as 1,3-Benzenediacetonitrile, α,α,α′,α′-tetramethyl-5-(1H-1,2,4-triazol-1-ylmethyl).

The dose of Anastrozole is up to 2 mg taken once a day. Anastrozole may be used in combination with up to about 5 mg/kg CpG ODN, preferably PF-3512676, 1018 ISS, Genasense, or IMOxine®, administered subcutaneously. In a preferred embodiment, PF3512676 is administered at a therapeutic dose of about 0.01 to 2.5 mg/kg, more preferably, about 0.05 to 1.0 mg/kg, and most preferably about 0.2 mg/kg, and the therapeutic combination regimen described above is optionally followed by a maintenance dose of up to about 5 mg/kg PF3512676, preferably about 0.01 to 2.5 mg/kg, more preferably, about 0.05 to 1.0 mg/kg, and most preferably about 0.2 mg/kg.

(dd) Exemestane (Aromasin®; Exemestane tablets) is supplied by Pfizer Inc, New York, N.Y. Exemestane is an irreversible steroidal aromatase inactivator, chemically described as 6-methylenandrosta-1,4-diene-3,17-dione. Exemestane is an irreversible, steroidal aromatase inactivator, structurally related to the natural substrate androstenedione. It acts as a false substrate for the aromatase enzyme, and is processed to an intermediate that binds irreversibly to the active site of the enzyme causing its inactivation, an effect also known as “suicide inhibition.” Exemestane significantly lowers circulating estrogen concentrations in postmenopausal women, but has no detectable effect on adrenal biosynthesis of corticosteroids or aldosterone. Exemestane has no effect on other enzymes involved in the steroidogenic pathway up to a concentration at least 600 times higher than that inhibiting the aromatase enzyme.

The recommended dose of exemestane is up to about 50 mg, in combination with up to about 5 mg/kg CpG ODN, preferably PF-3512676, 1018 ISS, Genasense, or IMOxine®, administered subcutaneously. In a preferred embodiment, PF3512676 is administered at a therapeutic dose of about 0.01 to 2.5 mg/kg, more preferably, about 0.05 to 1.0 mg/kg, and most preferably about 0.2 mg/kg, and the therapeutic combination regimen described above is optionally followed by a maintenance dose of up to about 5 mg/kg PF3512676, preferably about 0.01 to 2.5 mg/kg, more preferably, about 0.05 to 1.0 mg/kg, and most preferably about 0.2 mg/kg.

(dd) BiCNU® (Carmustine for injection) is supplied by Bristol Myers Squibb, Princeton, N.J. It is 1,3-bis(2-chloroethyl)-1-nitrosourea. Although it is generally agreed that carmustine alkylates DNA and RNA, it is not cross resistant with other alkylators. As with other nitrosoureas, it may also inhibit several key enzymatic processes by carbamoylation of amino acids in proteins.

The recommended dose of BiCNU® is up to about 200 mg/m², e.g., 150 to 200 mg/m². BiCNU® may be administered in combination with up to about 5 mg/kg CpG ODN, preferably PF-3512676, 1018 ISS, Genasense, or IMOxine®, administered subcutaneously. In a preferred embodiment, PF3512676 is administered at a therapeutic dose of about 0.01 to 2.5 mg/kg, more preferably, about 0.05 to 1.0 mg/kg, and most preferably about 0.2 mg/kg, and the therapeutic combination regimen described above is optionally followed by a maintenance dose of up to about 5 mg/kg PF3512676, preferably about 0.01 to 2.5 mg/kg, more preferably, about 0.05 to 1.0 mg/kg, and most preferably about 0.2 mg/kg.

(ee) Temozolomide (Temodar®) is supplied by Schering Corporation, Kenilworth, N.J. It is 3,4-dihydro-3-methyl-4-oxoimidazo[5,1-d]-as-tetrazine-8-carboxamide. Temozolomide is not directly active but undergoes rapid nonenzymatic conversion at physiologic pH to the reactive compound MTIC. The cytotoxicity of MTIC is thought to be primarily due to alkylation of DNA. Alkylation (methylation) occurs mainly at the O6 and N7 positions of guanine.

Temozolomide is administered at a dose of up to about 200 mg/m², alone or in combination with alpha-interferon, 5 MU/m², administered subcutaneously, in combination with up to about 5 mg/kg CpG ODN, preferably PF-3512676, 1018 ISS, Genasense, or IMOxine®, administered subcutaneously. In a preferred embodiment, PF3512676 is administered at a therapeutic dose of about 0.01 to 2.5 mg/kg, more preferably, about 0.05 to 1.0 mg/kg, and most preferably about 0.2 mg/kg, and the therapeutic combination regimen described above is optionally followed by a maintenance dose of up to about 5 mg/kg PF3512676, preferably about 0.01 to 2.5 mg/kg, more preferably, about 0.05 to 1.0 mg/kg, and most preferably about 0.2 mg/kg.

(ff) Leukeran (Chlorambucil) is supplied by GlaxoSmithKline, Research Triangle Park, N.C. It is a bifunctional alkylating agent of the nitrogen mustard type that has been found active against selected human neoplastic diseases. Chlorambucil is known chemically as 4-[bis(2-chloroethyl)amino]benzenebutanoic acid.

The usual oral dosage of chlorambucil is up to about 0.2 mg/kg, e.g., 0.1 to 0.2 mg/kg. This usually amounts to about 4 to 10 mg per day for the average patient. The entire daily dose may be given at one time. These dosages are for initiation of therapy or for short courses of treatment. The dosage must be carefully adjusted according to the response of the patient and must be reduced as soon as there is an abrupt fall in the white blood cell count. Patients with Hodgkin's disease usually require about 0.2 mg/kg daily, whereas patients with other lymphomas or chronic lymphocytic leukemia usually require only 0.1 mg/kg daily. Chlorambucil may be used in combination with up to about 5 mg/kg CpG ODN, preferably PF-3512676, 1018 ISS, Genasense, or IMOxine®, administered subcutaneously. In a preferred embodiment, PF3512676 is administered at a therapeutic dose of about 0.01 to 2.5 mg/kg, more preferably, about 0.05 to 1.0 mg/kg, and most preferably about 0.2 mg/kg, and the therapeutic combination regimen described above is optionally followed by a maintenance dose of up to about 5 mg/kg PF3512676, preferably about 0.01 to 2.5 mg/kg, more preferably, about 0.05 to 1.0 mg/kg, and most preferably about 0.2 mg/kg.

(gg) Fludarabine (Fludara; Fludarabine phosphate) is available from Berlex Laboratories, Inc. Fludarabine is a fluorinated nucleoside analog of the antiviral agent, vidarabine. The chemical name for fludarabine phosphate is 9H-Purin-6-amine, 2-fluoro-9-(5-0-phosphono-_-D-arabinofuranosyl). Fludarabine phosphate is rapidly dephosphorylated to 2-fluoro-ara-A and then phosphorylated intracellularly by deoxycytidine kinase to the active triphosphate, 2-fluoro-ara-ATP. This metabolite appears to act by inhibiting DNA polymerase alpha, ribonucleotide reductase and DNA primase, thus inhibiting DNA synthesis. The mechanism of action of this antimetabolite is not completely characterized and may be multi-faceted.

The recommended adult dose of Fludarabine is up to about 25 mg/m² in combination with up to about 5 mg/kg CpG ODN, preferably PF-3512676, 1018 ISS, Genasense, or IMOxine®, administered subcutaneously. In a preferred embodiment, PF3512676 is administered at a therapeutic dose of about 0.01 to 2.5 mg/kg, more preferably, about 0.05 to 1.0 mg/kg, and most preferably about 0.2 mg/kg, and the therapeutic combination regimen described above is optionally followed by a maintenance dose of up to about 5 mg/kg PF3512676, preferably about 0.01 to 2.5 mg/kg, more preferably, about 0.05 to 1.0 mg/kg, and most preferably about 0.2 mg/kg.

(hh) Rituximab (Rituxan) is available from Genentech, Inc, South San Francisco, Calif. Rituxan is a monoclonal antibody that targets B cells. Within the first 3 doses, B cells are rapidly depleted, with a general return to pre-treatment levels within 5 to 12 months after completion of therapy.

Rituximab may be used in combination with up to about 5 mg/kg CpG ODN, preferably PF-3512676, 1018 ISS, Genasense, or IMOxine®, administered subcutaneously. In a preferred embodiment, PF3612676 is administered at a therapeutic dose of about 0.01 to 2.5 mg/kg, more preferably, about 0.05 to 1.0 mg/kg, and most preferably about 0.2 mg/kg, and the therapeutic combination regimen described above is optionally followed by a maintenance dose of up to about 5 mg/kg PF3512676, preferably about 0.01 to 2.5 mg/kg, more preferably, about 0.05 to 1.0 mg/kg, and most preferably about 0.2 mg/kg.

(ii) Getuximab (Erbitux®) is supplied by Bristol Myers Squibb, Princeton, N.J. Cetuximab is a recombinant, human/mouse chimeric monoclonal antibody that binds specifically to the extracellular domain of the human epidermal growth factor receptor (EGFR). Cetuximab is composed of the Fv regions of a murine anti-EGFR antibody with human IgG1 heavy and kappa light chain constant regions and has an approximate molecular weight of 152 kDa. Cetuximab is produced in mammalian (murine myeloma) cell culture. Cetuximab binds specifically to the epidermal growth factor receptor (EGFR, HER1, c-ErbB-1) on both normal and tumor cells, and competitively inhibits the binding of epidermal growth factor (EGF) and other ligands, such as transforming growth factor-alpha. Binding of cetuximab to the EGFR blocks phosphorylation and activation of receptor-associated kinases, resulting in inhibition of cell growth, induction of apoptosis, and decreased matrix metalloproteinase and vascular endothelial growth factor production. The EGFR is a transmembrane glycoprotein that is a member of a subfamily of type I receptor tyrosine kinases including EGFR (HER1), HER2, HER3, and HER4. The EGFR is constitutively expressed in many normal epithelial tissues, including the skin and hair follicle. Over-expression of EGFR is also detected in many human cancers including those of the colon and rectum. In vitro assays and in vivo animal studies have shown that cetuximab inhibits the growth and survival of tumor cells that over-express the EGFR. No anti-tumor effects of cetuximab were observed in human tumor xenografts lacking EGFR expression. The addition of cetuximab to irinotecan or irinotecan plus 5-fluorouracil in animal studies resulted in an increase in antitumor effects compared to chemotherapy alone.

Cetuximab may be used alone or in combination with irinotecan, wherein the dose of irinotecan is as described hereinabove, and the dose of cetuximab is up to about 400 mg/m². Up to about 5 mg/kg CpG ODN is added to cetuximab monotherapy or combination therapy with irinotecan, preferably PF-3512676, 1018 ISS, Genasense, or IMOxine®, administered subcutaneously. In a preferred embodiment, PF3512676 is administered at a therapeutic dose of about 0.01 to 2.5 mg/kg, more preferably, about 0.05 to 1.0 mg/kg, and most preferably about 0.2 mg/kg, and the therapeutic combination regimen described above is optionally followed by a maintenance dose of up to about 5 mg/kg PF3512676, preferably about 0.01 to 2.5 mg/kg, more preferably, about 0.05 to 1.0 mg/kg, and most preferably about 0.2 mg/kg.

For the treatment of cancer, and in a preferred embodiment, for the treatment of SCLC, a CpG ODN, preferably PF-3512676, 1018 ISS, oblimersen (Genasense®) or IMOxine®, may be administered alone or as part of a treatment regimen in combination any of the following chemotherapeutics administered alone or as part of a chemotherapy combination regimen:

TABLE 1

| Agent | Dose |
|---|---|
| CpG ODN | Up to about 5.0 mg/kg |
| Cisplatin | Up to about 120 mg/m² |
| Carboplatin | Dosed according to Calvert formula |
| | with AUC up to about 8 (see above) |
| Etoposide | Up to about 250 mg/m² |
| Cyclophosphamide | Up to about 7grams/m2, preferably |
| | up to about 750 mg/m² or 500 mg/m² |
| Doxorubicin | Up to about 100 mg/m², preferably |
| | up to about 75 mg/m² |
| Vincristine | Up to about 30 mg/kg, e.g., 10-30 mg/kg |
| | or about 2 mg/m² |
| Irinotecan | Up to about 500 mg/m², and |
| | preferably up to about 350 mg/m² |

For the treatment of cancer, and in a preferred embodiment, for the treatment of melanoma, a CpG ODN, preferably PF-3512676, 1018 ISS, oblimersen (Genasense®) or IMOxine®, may be administered alone or as part of a treatment regimen in combination any of the following chemotherapeutics administered alone or as part of a chemotherapy combination regimen:

TABLE 2

| Agent | Dose |
|---|---|
| CpG ODN | Up to about 5.0 mg/kg |
| Dacarbazine | Up to about 500 mg/m², preferably up |
| | to about 250 mg/m² |
| Carmustine | Up to about 200 mg/m² |
| Cisplatin | Up to about 120 mg/m² |
| Vinblastine | Up to about 50 mg/m², preferably up |
| | to about 30 mg/m² |
| Tamoxifen | Up to about 80 mg, preferably up to |
| | about 40 mg |

For the treatment of cancer and in a preferred embodiment, for the treatment of NSCLC, a CpG ODN, preferably PF-3512676, 1018 ISS, oblimersen (Genasense®) or IMOxine®, may be administered alone or as part of a treatment regimen in combination any of the following chemotherapeutics administered alone or as part of a chemotherapy combination regimen:

TABLE 3

| Agent | Dose |
|---|---|
| CpG ODN | Up to about 5.0 mg/kg |
| Cisplatin | Up to about 120 mg/m² |
| Gemcitabine | Up to about 2500 mg/m², preferably up to |
| | about 1250 mg/m² |
| Carboplatin | Dosed according to Calvert formula with |
| | AUC up to about 8 (see above) |
| Paclitaxel | Up to about 250 mg/m², preferably up to |
| | about 225 mg/m² |
| Bevacizumab | Up to about 15 mg/kg |
| (Avastin ®) | |
| Docetaxel | Up to about 120 mg/m², preferably up to 100 mg/m² |
| Pemetrexed | Up to about 1000 mg/m², preferably up to |
| | 750 mg/m², most preferably 500 mg/m² |
| Vinorelbine | Up to about 50 mg/m2, preferably up to |
| | about 30 mg/m² |

For the treatment of cancer, and in a preferred embodiment, the treatment of breast cancer, a CpG ODN, preferably PF-3512676, 1018 ISS, oblimersen (Genasense®) or IMOxine®, may be administered alone or as part of a treatment regimen in combination any of the following chemotherapeutics administered alone or as part of a chemotherapy combination regimen:

TABLE 4

| Agent | Dose |
|---|---|
| CpG ODN | Up to about 5.0 mg/kg |
| Doxorubicin | UP to about 100 mg/m², preferably up to |
| | about 75 mg/m² |
| Epirubicin | Up to about 150 mg/m², preferably up to |
| | about 135 mg/m² or 120 mg/m² |
| Cyclophosphamide | Up to about 7 grams/m², preferably about |
| | 750 mg/m² to about 500 mg/m² |
| Methotrexate | Up to about 20 g/m² |
| 5-FU | Up to about 2600 mg/m², preferably up to |
| | about 600 mg/m² |
| Paclitaxel | Up to about 250 mg/m², preferably up to |
| | about 225 mg/m² |
| Docetaxel | Up to about 120 mg/m2, preferably up to |
| | 100 mg/m² |
| Gemcitabine | Up to about 2500 mg/m², preferably up to |
| | about 1250 mg/m² |
| Cisplatin | Up to about 120 mg/m² |
| Carboplatin | Dosed according to Calvert formula with |
| | AUC up to about 8 (see above) |
| Vinorelbine | Up to about 50 mg/m2, preferably up to |
| | about 30 mg/m² |
| Bevacizumab | Up to about 15 mg/kg |
| (Avastin ®) | |

For the treatment of cancer and preferably, colorectal cancer, a CpG ODN preferably PF-3512676, 1018 ISS, oblimersen (Genasense®) or IMOxine®, may be administered alone or as part of a treatment regimen in combination any of the following chemotherapeutics administered alone or as part of a chemotherapy combination regimen:

TABLE 5

| Agent | Dose |
|---|---|
| CpG ODN | Up to about 5.0 mg/kg |
| Irinotecan | Up to about 500 mg/m²preferably up to |
| | about 350 mg/m² |
| Oxaliplatin | Up to about 130 mg/m², preferably up to 85 mg/m² |
| 5-FU | Up to about 2600 mg/m², preferably up to |
| | about 600 mg/m² |
| Cetuximab | Up to about 400 mg/m² |
| (Erbitux ®) | |
| Bevacizumab | Up to about 15 mg/kg |
| (Avastin ®) | |

For the treatment of cancer, and preferably NHL, a CpG ODN, preferably PF-3512676, 1018 ISS, oblimersen (Genasense®) or IMOxine®, may be administered alone or as part of a treatment regimen in combination any of the following chemotherapeutics administered alone or as part of a chemotherapy combination regimen:

TABLE 6

| Agent | Dose |
|---|---|
| CpG ODN | Up to about 5.0 mg/kg |
| Rituximab | Up to about 500 mg/m² and preferably up to |
| | about 375 mg/m² |
| Cyolophosphamide | Up to about 7 grams/m² preferably about |
| | 750 mg/m² to about 500 mg/m² |
| Prednisolone | Up to about 5 mg/kg, and preferably up to |
| | about 1 mg/kg |
| Vincristine | Up to about 30 mg/kg, e.g., 10-30 mg/kg or |
| | about 2 mg/m² |

Other Chemotherapeutic Agents:

In general, the CpG ODNs of the present invention, and particularly, PF3512676, 1018 ISS, or IMOxine®, may be used in combination with any number of chemotherapeutic agents for the treatment of a variety of cancers. Any of the agents described above may be administered in combination with CpG ODNs, and optionally one or more additional chemotherapeutic agents. A non-limiting list of chemotherapeutic agents, organized according to approximate mechanism of action is provided below:

(1) Anti-angiogenesis agents, include but are not limited to the following agents, such as EGF inhibitor, EGFR inhibitors, VEGF inhibitors, VEGFR inhibitors, TIE2 inhibitors, IGF1R inhibitors, COX-II (cyclooxygenase II) inhibitors, MMP-2 (matrix-metalloproteinase 2) inhibitors, and MMP-9 (matrix-metalloproteinase 9) inhibitors. Anti-angiogenesis agents, such as MMP-2 (matrix-metalloproteinase 2) inhibitors, MMP-9 (matrix-metalloproteinase 9) inhibitors, and COX-II (cyclooxygenase II) inhibitors, can be used in conjunction with an immunostimulatory ODN in the methods and pharmaceutical compositions described herein. Examples of useful COX-II inhibitors include CELEBREX™ (celecoxib), Bextra (valdecoxib), paracoxib, Vioxx (rofecoxib), and Arcoxia (etoricoxib). Examples of useful matrix metalloproteinase inhibitors are described in WO 96/33172 (published Oct. 24, 1996), WO 96/27583 (published Mar. 7, 1996), European Patent Application No. 97304971.1 (filed Jul. 8, 1997), European Patent Application No. 99308617.2 (filed Oct. 29, 1999), WO 98/07697 (published Feb. 26, 1998), WO 98/03516 (published Jan. 29, 1998), WO 98/34918 (published Aug. 13, 1998), WO 98/34915 (published Aug. 13, 1998), WO 98/33768 (published Aug. 6, 1998), WO 98/30566 (published Jul. 16, 1998), European Patent Publication 606,046 (published Jul. 13, 1994), European Patent Publication 931,788 (published Jul. 28, 1999), WO 90/05719 (published May 331, 1990), WO 99/52910 (published Oct. 21, 1999), WO 99/52889 (published Oct. 21, 1999), WO 99/29667 (published Jun. 17, 1999), PCT International Application No. PCT/IB98/01113 (filed Jul. 21, 1998), European Patent Application No. 99302232.1 (filed Mar. 25, 1999), Great Britain patent application number 9912961.1 (filed Jun. 3, 1999), U.S. Provisional Application No. 60/148,464 (filed Aug. 12, 1999), U.S. Pat. No. 5,863,949 (issued Jan. 26, 1999), U.S. Pat. No. 5,861,510 (issued Jan. 19, 1999), and European Patent Publication 780,386 (published Jun. 25, 1997), all of which are herein incorporated by reference in their entirety. Preferred MMP-2 and MMP-9 inhibitors are those that have little or no activity inhibiting MMP-1. More preferred, are those that selectively inhibit MMP-2 and/or MMP-9 relative to the other matrix-metalloproteinases (i.e. MMP-1, MMP-3, MMP-4, MMP-5, MMP-6, MMP-7, MMP-8, MMP-10, MMP-11, MMP-12, and MMP-13). Some specific examples of MMP inhibitors useful in combination with the CpG ODN of the present invention are AG-3340, RO 32-3555, RS 13-0830, and the compounds recited in the following list: 3-[[4-(4-fluoro-phenoxy)-benzenesulfonyl]-(1-hydroxycarbamoyl-cyclopentyl)-amino]-propionic acid; 3-exo-3-[4-(4-fluoro-phenoxy)-benzenesulfonylamino]-8-oxa-bicyclo[3.2.1]octane-3-carboxylic acid hydroxyamide; (2R,3R) 1-[4-(2-chloro-4-fluoro-benzyloxy)-benzenesulfonyl]-3-hydroxy-3-methyl-piperidine-2-carboxylic acid hydroxyamide; 4-[4-(4-fluoro-phenoxy)-benzenesulfonylamino]-tetrahydro-pyran-4-carboxylic acid hydroxyamide; 3-[[4-(4-fluoro-phenoxy)-benzenesulfonyl]-(1-hydroxycarbamoyl-cyclobutyl)-amino]-propionic acid; 4-[4-(4-chloro-phenoxy)-benzenesulfonylamino]-tetrahydro-pyran-4-carboxylic acid hydroxyamide; 3-[4-(4-chloro-phenoxy)-benzenesulfonylamino]-tetrahydro-pyran-3-carboxylic acid hydroxyamide; (2R,3R) 1-[4-(4-fluoro-2-methyl-benzyloxy)-benzenesulfonyl]-3-hydroxy-3-methyl-piperidine-2-carboxylic acid hydroxyamide; 3-[[4-(4-fluoro-phenoxy)-benzenesulfonyl]-(1-hydroxycarbamoyl-1-methyl-ethyl)-amino]-propionic acid; 3-[[4-(4-fluoro-phenoxy)-benzenesulfonyl]-(4-hydroxycarbamoyl-tetrahydro-pyran-4-yl)-amino]-propionic acid; 3-exo-3-[4-(4-chloro-phenoxy)-benzenesulfonylamino]-8-oxa-bicyclo[3.2.1]octane-3-carboxylic acid hydroxyamide; 3-endo-3-[4-(4-fluoro-phenoxy)-benzenesulfonylamino]-8-oxa-bicyclo[3.2.1]octane-3-carboxylic acid hydroxyamide; and 3-[4-(4-fluoro-phenoxy)-benzenesulfonylamino]-tetrahydro-furan-3-carboxylic acid hydroxyamide; and pharmaceutically acceptable salts, solvates and prodrugs of said compounds.

Additional VEGF inhibitors include CP-547,632 (Pfizer Inc., NY, USA), AG13736 (Pfizer Inc.), ZD-6474 (AstraZeneca), AEE788 (Novartis), AZD-2171), VEGF Trap (Regeneron/Aventis), Vatalanib (also known as PTK-787, ZK-222584: Novartis & Schering AG), Macugen (pegaptanib octasodium, NX-1838, EYE-001, Pfizer Inc./Gilead/Eyetech), IM862 (Cytran Inc. of Kirkland, Wash., USA); and angiozyme, a synthetic ribozyme from Ribozyme (Boulder, Colo.) and Chiron (Emeryville, Calif.) and combinations thereof. VEGF inhibitors useful in the practice of the present invention are disclosed in U.S. Pat. Nos. 6,534,524 and 6,235,764, both of which are incorporated in their entirety for all purposed. Particularly preferred VEGF inhibitors include CP-547,632, AG13736, Vatalanib, Macugen and combinations thereof. Additional VEGF inhibitors are described in, for example in WO 99/24440 (published May 20, 1999), PCT International Application PCT/IB99/00797 (filed May 3, 1999), in WO 95/21613 (published Aug. 17, 1995), WO 99/61422 (published Dec. 2, 1999), U.S. Pat. No. 6,534,524 (discloses AG13736), U.S. Pat. No. 5,834,504 (issued Nov. 10, 1998), WO 98/50356 (published Nov. 12, 1998), U.S. Pat. No. 5,883,113 (issued Mar. 16, 1999), U.S. Pat. No. 5,886,020 (issued Mar. 23, 1999), U.S. Pat. No. 5,792,783 (issued Aug. 11, 1998), U.S. Pat. No. 6,653,308 (issued Nov. 25, 2003), WO 99/10349 (published Mar. 4, 1999), WO 97/32856 (published Sep. 12, 1997), WO 97/22596 (published Jun. 26, 1997), WO 98/54093 (published Dec. 3, 1998), WO 98/02438 (published Jan. 22, 1998), WO 99/16755 (published Apr. 8, 1999), and WO 98/02437 (published Jan. 22, 1998), all of which are herein incorporated by reference in their entirety.

Other antiproliferative agents that may be used with the compounds of the present invention include inhibitors of the enzyme farnesyl protein transferase and inhibitors of the receptor tyrosine kinase PDGFr, including the compounds disclosed and claimed in the following U.S. patent application Ser Nos. 09/221,946 (filed Dec. 28, 1998); 09/454,058 (filed Dec. 2, 1999); 09/501,163 (filed Feb. 9, 2000); 09/539,930 (filed Mar. 31, 2000); 09/202,796 (filed May 22, 1997); 09/384,339 (filed Aug. 26, 1999); and 09/383,755 (filed Aug. 26, 1999); and the compounds disclosed and claimed in the following U.S. provisional patent applications: 60/168,207 (filed Nov. 30, 1999); 60/170,119 (filed Dec. 10, 1999); 60/177,718 (filed Jan. 21, 2000); 60/168,217 (filed Nov. 30, 1999), and 60/200,834 (filed May 1, 2000). Each of the foregoing patent applications and provisional patent applications is herein incorporated by reference in their entirety.

PDGRr inhibitors include but not limited to those disclosed international patent application publication number WO01/40217, published Jul. 7, 2001 and international patent application publication number WO2004/020431, published Mar. 11, 2004, the contents of which are incorporated in their entirety for all purposes. Preferred PDGFr inhibitors include Pfizer's CP-673,451 and CP-868,596 and its pharmaceutically acceptable salts. Preferred GARF inhibitors include Pfizer's AG-2037 (pelitrexol and its pharmaceutically acceptable salts. GARF inhibitors useful in the practice of the present invention are disclosed in U.S. Pat. No. 5,608,082 which is incorporated in its entirety for all purposed.

(2) Tyrosine kinase inhibitors, include targeted agents used in conjunction with a CpG ODN and pharmaceutical compositions described herein include EGFr inhibitors such as Iressa® (gefitinib, AstraZeneca), Tarceva (erlotinib or OSI-774, OSI Pharmaceuticals Inc.), Erbitux (cetuximab, Imclone Pharmaceuticals, Inc.), EMD-7200 (Merck AG), ABX-EGF (Amgen Inc. and Abgenix Inc.), HR3 (Cuban Government), IgA antibodies (University of Erlangen-Nuremberg), TP-38 (IVAX), EGFR fusion protein, EGF-vaccine, anti-EGFr immunoliposomes (Hermes Biosciences Inc.) and combinations thereof. Preferred EGFr inhibitors include gefitinib, cetuximab, erlotinib and combinations thereof.

The present invention also relates to combinations including anti-tumor agents selected from pan erb receptor inhibitors or ErbB2 receptor inhibitors, such as CP-724,714 (Pfizer, Inc.), CI-1033 (canertinib, Pfizer, Inc.), Herceptin (trastuzumab, Genentech Inc.), Omitarg (2C4, pertuzumab, Genentech Inc.), TAK-165 (Takeda), GW-572016 (Ionafarnib, GlaxoSmithKline), GW-282974 (GlaxoSmithKline), EKB-569 (Wyeth), PKI-166 (Novartis), dHER2 (HER2Vaccine, Corixa and GlaxoSmithKline), APC8024 (HER2Vaccine, Dendreon), anti-HER2/neu bispecific antibody (Decof Cancer Center), B7.her2.IgG3 (Agensys), AS HER2 (Research Institute for Rad Biology & Medicine), trifuntional bispecific antibodies (University of Munich) and mAB AR-209 (Aronex Pharmaceuticals Inc) and mAB 2B-1 (Chiron) and combinations thereof. Preferred erb selective anti-tumor agents include Trastuzumab, TAK-165, CP-724,714, ABX-EGF, HER3 and combinations thereof. Preferred pan erbb receptor inhibitors include GW572016, CI-1033, PF-299804, EKB-569, and Omitarg and combinations thereof. Additional erbB2 inhibitors include those described in WO 98/02434 (published Jan. 22, 1998), WO 99/35146 (published Jul. 15, 1999), WO 99/35132 (published Jul. 15, 1999), WO 98/02437 (published Jan. 22, 1998), WO 97/13760 (published Apr. 17, 1997), WO 95/19970 (published Jul. 27, 1995), U.S. Pat. No. 5,587,458 (issued Dec. 24, 1996), and U.S. Pat. No. 5,877,305 (issued Mar. 2, 1999), each of which is herein incorporated by reference in its entirety. ErbB2 receptor inhibitors useful in the present invention are also described in U.S. Pat. Nos. 6,465,449, and 6,284,764, and International Application No. WO 2001/98277 each of which are herein incorporated by reference in their entirety.

Also included in the category of tyrosine kinase inhibitors are ErbB2 receptor inhibitors, such as GW-282974 (Glaxo Wellcome plc), and the monoclonal antibodies AR-209 (Aronex Pharmaceuticals Inc. of The Woodlands, Tex., USA) and 2B-1 (Chiron), which may be administered in combination with a CpG ODN. Such erbB2 inhibitors include 2C4 and pertuzumab. Such erbB2 inhibitors include those described in WO 98/02434 (published Jan. 22, 1998), WO 99/35146 (published Jul. 15, 1999), WO 99/35132 (published Jul. 15, 1999), WO 98/02437 (published Jan. 22, 1998), WO 97/13760 (published Apr. 17, 1997), WO 95/19970 (published Jul. 27, 1995), U.S. Pat. No. 5,587,458 (issued Dec. 24, 1996), and U.S. Pat. No. 5,877,305 (issued Mar. 2, 1999), each of which is herein incorporated by reference in its entirety. ErbB2 receptor inhibitors useful in the present invention are also described in U.S. Provisional Application No. 60/117,341, filed Jan. 27, 1999, and in U.S. Provisional Application No. 60/117,346, filed Jan. 27, 1999, both of which are herein incorporated by reference in their entirety. Other erbb2 receptor inhibitors include TAK-165 (Takeda) and GW-572016 (Glaxo-Wellcome).

Various other compounds, such as styrene derivatives, have also been shown to possess tyrosine kinase inhibitory properties, and some of tyrosine kinase inhibitors have been identified as erbB2 receptor inhibitors. More recently, five European patent publications, namely EP 0 566 226 A1 (published Oct. 20, 1993), EP 0 602 851 A1 (published Jun. 22, 1994), EP 0 635 507 A1 (published Jan. 25, 1995), EP 0 635 498 A1 (published Jan. 25, 1995), and EP 0 520 722 A1 (published Dec. 30, 1992), refer to certain bicyclic derivatives, in particular quinazoline derivatives, as possessing anti-cancer properties that result from their tyrosine kinase inhibitory properties. Also, World Patent Application WO 92/20642 (published Nov. 26, 1992), refers to certain bis-mono and bicyclic aryl and heteroaryl compounds as tyrosine kinase inhibitors that are useful in inhibiting abnormal cell proliferation. World Patent Applications WO96/16960 (published Jun. 6, 1996), WO 96/09294 (published Mar. 6, 1996), WO 97/30034 (published Aug. 21, 1997), WO 98/02434 (published Jan. 22, 1998), WO 98/02437 (published Jan. 22, 1998), and WO 98/02438 (published Jan. 22, 1998), also refer to substituted bicyclic heteroaromatic derivatives as tyrosine kinase inhibitors that are useful for the same purpose. Other patent applications that refer to anti-cancer compounds are World Patent Application WO00/44728 (published Aug. 3, 2000), EP 1029853A1 (published Aug. 23, 2000), and WO01/98277 (published Dec. 12, 2001) all of which are incorporated herein by reference in their entirety.

Other antiproliferative agents that may be used with the CpG ODN of the present invention include inhibitors of the enzyme farnesyl protein transferase and inhibitors of the receptor tyrosine kinase PDGFr, including the compounds disclosed and claimed in the following U.S. patent application Ser. Nos. 09/221,946 (filed Dec. 28, 1998); 09/454,058 (filed Dec. 2, 1999); 09/501163 (filed Feb. 9, 2000); 09/539,930 (filed Mar. 31, 2000); 09/202,796 (filed May 22, 1997); 09/384,339 (filed Aug. 26, 1999); and 09/383,755 (filed Aug. 26, 1999); and the compounds disclosed and claimed in the following United States provisional patent applications: 60/168,207 (filed Nov. 30, 1999); 60/170,119 (filed Dec. 10, 1999); 60/177718 (filed Jan. 21, 2000); 60/168,217 (filed Nov. 30, 1999), and 60/200,834 (filed May 1, 2000). Each of the foregoing patent applications and provisional patent applications is herein incorporated by reference in their entirety.

CpG ODNs may also be used in combination with cytotoxic agents, e.g., one or more selected from the group consisting of a camptothecin, irinotecan HCl (Camptosar), edotecarin, epirubicin (Ellence), docetaxel (Docetaxel), rituximab (Rituxan), Cetuximab, and combinations thereof.

(3) Alkylating agents include, but are not limited to, nitrogen mustard N-oxide, cyclophosphamide, ifosfamide, melphalan, busulfan, mitobronitol, carboquone, thiotepa, ranimustine, nimustine, temozolomide, AMD-473, altretamine, AP-5280, apaziquone, brostallicin, bendamustine, carmustine, estramustine, fotemustine, glufosfamide, ifosfamide, KW-2170, mafosfamide, and mitolactol; platinum-coordinated alkylating compounds include but are not limited to, cisplatin, carboplatin, eptaplatin, lobaplatin, nedaplatin, oxaliplatin or satrplatin;

(4) Antimetabolites include but are not limited to, methotrexate, 6-mercaptopurine riboside, mercaptopurine, 5-fluorouracil (5-FU) alone or in combination with leucovorin, tegafur, UFT, doxifluridine, carmofur, cytarabine, cytarabine ocfosfate, enocitabine, S-1, Gemcitabine, fludarabin, 5-azacitidine, capecitabine, cladribine, clofarabine, decitabine, eflornithine, ethynylcytidine, cytosine arabinoside, hydroxyurea, TS-1, melphalan, nelarabine, nolatrexed, ocfosfate, disodium premetrexed, pentostatin, pelitrexol, raltitrexed, triapine, trimetrexate, vidarabine, vincristine, vinorelbine; or for example, one of the preferred anti-metabolites disclosed in European Patent Application No. 239362 such as N-(5-[N-(3,4-dihydro-2-methyl-4-oxoquinazolin-6-ylmethyl)-N-methylamino]-2-thenoyl)-L-glutamic acid;

(5) Antibiotics include but are not limited to: aclarubicin, actinomycin D, amrubicin, annamycin, bleomycin, daunorubicin, doxorubicin, elsamitrucin, epirubicin, galarubicin, idarubicin, mitomycin C, nemorubicin, neocarzinostatin, peplomycin, pirarubicin, rebeccamycin, stimalamer, streptozocin, valrubicin or zinostatin;

(6) Hormonal therapy agents, e.g., exemestane (Aromasin), Lupron, anastrozole (Arimidex), doxercalciferol, fadrozole, formestane, anti-estrogens such as tamoxifen citrate (Nolvadex) and fulvestrant, Trelstar, toremifene, raloxifene, lasofoxifene, letrozole (Femara), or anti-androgens such as bicalutamide, flutamide, mifepristone, nilutamide, Casodex® (4′-cyano-3-(4-fluorophenylsulphonyl)-2-hydroxy-2-methyl-3′-(trifluoromethyl)propionanilide), lueoprorelin (Lupron), gosrelin, doxercalciferol, Abraelix, Treistar, and combinations thereof;

(7) Plant derived anti-tumor substances include for example those selected from mitotic inhibitors, for example vinblastine, docetaxel (Docetaxel) and Paclitaxel®;

(8) Cytotoxic topoisomerase inhibiting agents include one or more agents selected from the group consisting of aclarubicin, amonafide, belotecan, camptothecin, 10-hydroxycamptothecin, 9-aminocamptothecin, diflomotecan, irinotecan HCl (Camptosar), edotecarin, epirubicin (Ellence), etoposide, exatecan, gimatecan, lurtotecan, mitoxantrone, pirarubicin, pixantrone, rubitecan, sobuzoxane, SN-38, tafluposide, and topotecan, orathecin (Supergen), exatecan (Daiichi), BN-80915 (Roche) and combinations thereof.

(9) Immunologicals include interferons and numerous other immune enhancing agents. Interferons include interferon alpha, interferon alpha-2a, interferon, alpha-2b, interferon beta, interferon gamma-1a or interferon gamma-n1. Other agents include filgrastim, lentinan, sizofilan, TheraCys, ubenimex, WF-10, aldesleukin, alemtuzumab, BAM-002, dacarbazine, daclizumab, denileukin, gemtuzumab ozogamicin, ibritumomab, imiquimod, lenograstim, lentinan, melanoma vaccine (Corixa), molgramostim, OncoVAX-CL, sargramostim, tasonermin, tecleukin, thymalasin, tositumomab, Virulizin, Z-100, epratuzumab, mitumomab, oregovomab, pemtumomab, Provenge, and combinations thereof;

(10) Biological response modifiers are agents that modify defense mechanisms of living organisms or biological responses, such as survival, growth, or differentiation of tissue cells to direct them to have anti-tumor activity. Such agents include krestin, lentinan, sizofuran, picibanil, or ubenimex.

(11) Other antitumor agents include alitretinoin, ampligen, atrasentan bexarotene, bortezomib. Bosentan, calcitriol, exisulind, finasteride, fotemustine, ibandronic acid, miltefosine, mitoxantrone, 1-asparaginase, procarbazine, dacarbazine, hydroxycarbamide, pegaspargase, pentostatin, tazarotene, TLK-286, Velcade, or tretinoin. Additionally, other anti-tumor agents may be selected from the following agents, BAY-43-9006 (Onyx Pharmaceuticals Inc.), Genasense (augmerosen, Genta), Panitumumab (Abgenix/Amgen), Zevalin (Schering), Bexxar (Corixa/GlaxoSmithKline), Abarelix, Pemetrexed, EPO 906 (Novartis), discodermolide (XAA-296), ABT-510 (Abbott), Neovastat (Aeterna), enzastaurin (Eli Lilly), Combrestatin A4P (Oxigene), ZD-6126 (AstraZeneca), flavopiridol (Aventis), CYC-202 (Cyclacel), AVE-8062 (Aventis), DMXAA (Roche/Antisoma), Thymitaq (Eximias), Temodar (temozolomide, Schering Plough) and Revilimd (Celegene) and combinations thereof.

Other anti-tumor agents may be selected from the following agents, CyPat (cyproterone acetate), Histerelin (histrelin acetate), Plenaixis (abarelix depot), Atrasentan (ABT-627), Satraplatin (JM-216), thalomid (Thalidomide), Theratope, Temilifene (DPPE), ABI-007 (Paclitaxel®), Evista (raloxifene), Operia (lasofaxifene), Atamestane (Biomed-777), Xyotax (polyglutamate Paclitaxel®), Targetin (bexarotine) and combinations thereof. Additionally, other anti-tumor agents may be selected from the following agents, Trizaone (tirapazamine), Aposyn (exisulind), Nevastat (AE-941), Ceplene (histamine dihydrochloride), Orathecin (rubitecan), Virulizin, Gastrimmune (G17DT), DX-8951f (exatecan mesylate), Onconase (ranpirnase), BEC2 (mitumoab), Xcytrin (motexafin gadolinium) and combinations thereof. Further anti-tumor agents may selected from the following agents, CeaVac (CEA), NeuTrexin (trimetresate glucuronate) and combinations thereof. Additional anti-tumor agents may selected from the following agents, OvaRex (oregovomab), Osidem (IDM-1), and combinations thereof. Additional anti-tumor agents may selected from the following agents, Advexin (ING 201), Tirazone (tirapazamine), and combinations thereof. Additional anti-tumor agents may selected from the following agents, RSR13 (efaproxiral), Cotara (131I chTNT 1/b), NBI-3001 (IL-4) and combinations thereof. Additional anti-tumor agents may selected from the following agents, Canvaxin, GMK vaccine, Oncophage (HSPPC-96), PEG Interon A, Taxoprexin (DHA/paciltaxel) and combinations thereof. Other preferred anti-tumor agents include Pfizer's MEK1/2 inhibitor PD325901, Array Biopharm's MEK inhibitor ARRY-142886, Bristol Myers' CDK2 inhibitor BMS-387,032, Pfizer's CDK inhibitor PD0332991 and AstraZeneca's AXD-5438 and combinations thereof. Additionally, mTOR inhibitors may also be utilized such as CCI-779 (Wyeth) and rapamycin derivatives RAD001 (Novartis) and AP-23573 (Ariad), HDAC inhibitors SAHA (Merck Inc./Aton Pharmaceuticals) and combinations thereof. Additional anti-tumor agents include aurora 2 inhibitor VX-680 (Vertex), Chk1/2 inhibitor XL844 (Exilixis).

(12) Other anti-angiogenic compounds include acitretin, fenretinide, thalidomide, zoledronic acid, angiostatin, aplidine, cilengtide, combretastatin A-4, endostatin, halofuginone, rebimastat, removab, Revlimid, squalamine, ukrain and Vitaxin;

(13) Platinum-coordinated compounds include but are not limited to, cisplatin, carboplatin, nedaplatin, or oxaliplatin;

(14) Camptothecin derivatives include but are not limited to camptothecin, 10-hydroxycamptothecin, 9-aminocamptothecin, irinotecan, SN-38, edotecarin, and topotecan;

(15) Antibodies include Trastuzumab, Cetuximab, Bevacizumab™, Pertuzumab or Rituximab. Specific IGF1R antibodies that can be used in the present invention include those described in International Patent Application No. WO 2002/053596, which is herein incorporated by reference in its entirety. Specific CD40 antibodies that can be used in the present invention include those described in International Patent Application No. WO 2003/040170 which is herein incorporated by reference in its entirety.

(16) Gene therapy agents may also be employed as anti-tumor agents such as TNFerade (GeneVec), which express TNFalpha in response to radiotherapy.

(17) Other antitumor agents include mitoxantrone, I-asparaginase, procarbazine, dacarbazine, hydroxycarbamide, pentostatin, or tretinoin;

(18) Palliative agents: The present invention also encompasses the administration of other therapeutic agents in addition to the first and second components, either concurrently with one or more of those components, or sequentially. Such therapeutic agents include analgesics, cancer vaccines, anti-vascular agents, anti-proliferative agents, anti-emetic agents, and anti-diarrhea agents. Preferred anti-emetic agents include ondansetron hydrochloride, granisetron hydrochloride, and metoclopramide. Preferred anti-diarrhea agents include diphenoxylate and atropine (LOMOTIL), loperamide (IMODIUM), and octreotide (SANDOSTATIN). Further, the invention provides a CpG ODN of the present invention alone or in combination with one or more supportive care products, e.g., a product selected from the group consisting of Filgrastim (Neupogen), ondansetron (Zofran), Fragmin, Procrit, Aloxi, Emend, or combinations thereof. Such conjoint treatment may be achieved by way of the simultaneous, sequential or separate dosing of the individual components of the treatment.

One or more of the aforementioned chemotherapeutic agents may be used in combination with a CpG ODN, such as PF3512676, 1018 ISS, or IMOxine®, in combination with one or more additional therapeutic regimens, such as radiotherapy or stem cell-based therapy.

Radiation therapy can be co-administered with CpG ODN/chemotherapeutic agent combination therapy. The dose and regimen for radiotherapy can be readily determined by one skilled in the art and is based on the stage of the disease, and other factors well-known in the art. Radiation may be administered in a variety of fashions. For example, radiation may be electromagnetic or particulate in nature. Electromagnetic radiation useful in the practice of this invention includes, but is not limited, to x-rays and gamma rays. In a preferable embodiment, supervoltage x-rays x-rays>=4 MeV) may be used in the practice of this invention. Particulate radiation useful in the practice of this invention includes, but is not limited to, electron beams, protons beams, neutron beams, alpha particles, and negative pi mesons. The radiation may be delivered using conventional radiological treatment apparatus and methods, and by intraoperative and stereotactic methods. Additional discussion regarding radiation treatments suitable for use in the practice of this invention may be found throughout Steven A, Leibel et al., Textbook of Radiation Oncology (1998) (publ. W. B. Saunders Company), and particularly in Chapters 13 and 14. Radiation may also be delivered by other methods such as targeted delivery, for example by radioactive “seeds,” or by systemic delivery of targeted radioactive conjugates. J. Padawer et al., Combined Treatment with Radioestradiol lucanthone in Mouse C3HBA Mammary Adenocarcinoma and with Estradiol lucanthone in an Estrogen Bioassay, Int. J. Radiat. Oncol. Biol. Phys. 7:347-357 (1981). Other radiation delivery methods may be used in the practice of this invention.

The amount of radiation delivered to the desired treatment volume may be variable. In a preferable embodiment, radiation may be administered in amount effective to cause the arrest or regression of the cancer, in combination with a compound of formula 1 and pharmaceutical compositions described herein. In a more preferable embodiment, radiation is administered in at least about 1 Gray (Gy) fractions at least once every other day to a treatment volume, still more preferably radiation is administered in at least about 2 Gray (Gy) fractions at least once per day to a treatment volume, even more preferably radiation is administered in at least about 2 Gray (Gy) fractions at least once per day to a treatment volume for five consecutive days per week. In a more preferable embodiment, radiation is administered in 3 Gy fractions every other day, three times per week to a treatment volume. In yet another more preferable embodiment, a total of at least about 20 Gy, still more preferably at least about 30 Gy, most preferably at least about 60 Gy of radiation is administered to a host in need thereof. In one more preferred embodiment of the present invention 14 GY radiation is administered. In another more preferred embodiment of the present invention 10 GY radiation is administered. In another more preferred embodiment of the present invention 7 GY radiation is administered.

The chemotherapeutic agent-CpG ODN therapy combination disclosed herein can be combined with stem cell transplantation to provide a therapeutic benefit to a patient afflicted with cancer. Stem cell transplantation may be performed according to the methods known in the art. Some such methods are described in Appelbaum in Harrison's Principles of Internal Medicine, Chapter 14, Braunwald et al., Eds., 15^{th} ed., McGraw-Hill Professional (2001), which is hereby incorporated herein by reference. Thus, the methods of the present invention relate to the treatment of cancer in a mammal who has undergone stem cell transplantation, which methods comprise administering to the mammal an amount of a human chemotherapeutic agent in combination with CpG ODN, which chemotherapeutic agent-CpG ODN therapy combination is effective in treating the cancer in further combination with stem cell transplantation.

Where the method comprises stem cell transplant, the first dose of the chemotherapeutic agent-CpG ODN therapy agent combination can be administered after the immune system of the mammal has recovered from transplantation, for example, in the period of from one to 12 months post transplantation. In certain embodiments, the first dose is administered in the period of from one to three, or one to four months post transplantation. The patient may undergo stem cell transplantation and preparatory treatment(s).

The invention also relates to a method for the treatment of cancer in a mammal comprising the steps of (i) performing stem cell transplantation in the mammal, and (ii) administering an effective amount of a human chemotherapeutic agent in combination with an effective amount of CpG ODN. Preferably, the mammal is a human. Stem cell transplantation may be allogeneic or autologous stem cell transplantation. Further, cell transplantation encompasses adoptive transfer of lymphocytes, either from the same patient and/or from a HLA-matched donor.

Further, the methods of the invention can be combined with radiation therapy and stem cell transplant, and any combination of any of the treatments described herein, known in the art, or to be developed in the future.

Cancer Types

Combination of chemotherapeutic agent and CpG ODN is useful for treatment of primary and secondary (i.e., metastatic) cancers. More specifically, among many potential treatment options, CpG ODN and chemotherapeutic agent combination therapy can be used to treat renal cell carcinoma, breast cancer, colorectal cancer, ovarian cancer, non-small cell lung cancer, melanoma, cutaneous T-cell lymphoma, and NHL (including indolent and aggressive), among many others. While these cancers are preferred, the present invention relates to treatment of a wide variety of malignant cell proliferative disorders, including, but not limited to carcinomas and sarcomas. Further examples include Kaposi's sarcoma, erythroblastoma, mesothelioma, hepatobiliary (hepatic and biliary duct), a primary or secondary brain tumor, lung cancer (NSCLC and SCLC), bone cancer, skin cancer, cancer of the head or neck, cutaneous or intraocular melanoma, bone cancers, cancer of the anal region, stomach cancer, gastrointestinal (gastric, colorectal, and duodenal) cancer, colon cancers, uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, prostate cancers, cancer of the penis, testicular cancer, chronic or acute myeloid leukemia, chronic or acute lymphocytic leukemia, lymphocytic lymphomas, cancer of the bladder, cancer of the kidney or ureter, carcinoma of the renal pelvis, pancreatic cancers, neoplasms of the central nervous system (CNS) including primary or secondary CNS tumor, primary CNS lymphoma, spinal axis tumors, brain stem glioma, glioblastoma, meningioma, myoblastoma, astrocytoma, pituitary adenoma, adrenocortical cancer, gall bladder cancer, multiple myeloma, cholangiocarcinoma, fibrosarcoma, neuroblastoma, retinoblastoma, or a combination of one or more of the foregoing cancers.

In a preferred embodiment, the combination therapies of the invention are used in the treatment of a cancer selected from NSCLC, SCLC, melanoma, NHL, CTCL, and breast cancer.

In a further preferred embodiment, the invention provides a method of treating or preventing cancer, including but not limited to NSCLC, SCLC, melanoma, NHL, CTCL, and breast cancer, in a patient in need of such treatment, wherein the method comprises administering to the patient simultaneously, semi-simultaneously, separately or sequentially a therapeutically effective amount of a CpG ODN in combination with a therapeutically effective amount of a chemotherapeutic agent selected from the group consisting of 5-(5-fluoro-2-oxo-1,2-dihydroindol-(3Z)-ylidenemethyl)-2,4-dimethyl-1H-pyrrole-3-carboxylic acid (2-diethylaminoethyl)-amide, pemetrexed, gem citabine, paclitaxel, bevacizumab, carboplatin, Erlotinib, and combinations thereof and optionally (b) administering to the patient a maintenance regimen comprising a maintenance dose of a CpG ODN. In a preferred embodiment the CpG ODN is selected from the group consisting of PF3512676, 1018 ISS, and IMOxine®, and most preferably the CpG ODN is PF3512676. Moreover, the invention also provides a method of treating or preventing cancer as described herein wherein the chemotherapeutic agent is selected from the group consisting of 5-(5-fluoro-2-oxo-1,2-dihydroindol-(3Z)-ylidenemethyl)-2,4-dimethyl-1H-pyrrole-3-carboxylic acid (2-diethylaminoethyl)-amide, Pemetrexed, Gemcitabine, Bevacizumab™, carboplatin, Erlotinib, and combinations thereof, and said therapeutic regimen further comprises administering cisplatin. The invention also specifically contemplates a method of treatment that involves chemotherapy and immunotherapy using a CpG ODN as described above, wherein the method of treatment also includes one or more of surgery, radiation therapy, or a combination thereof.

Specifically, the invention provides a method of treating or preventing non-small cell lung cancer (NSCLC) in a patient in need of such treatment, wherein the method comprises (a) a therapeutic regimen comprising administering to the patient simultaneously, semi-simultaneously, separately or sequentially a therapeutically effective amount of a CpG ODN in combination with a therapeutically effective amount of a chemotherapeutic agent selected from the group consisting of 5-(5-fluoro-2-oxo-1,2-dihydroindol-(3Z)-ylidenemethyl)-2,4-dimethyl-1H-pyrrole-3-carboxylic acid (2-diethylaminoethyl)-amide, paclitaxel, docetaxel, gemcitabine, vinorelbine, irinotecan, pemetrexed, mitomycin, vincristine, vinblastine, vindesine, cisplatin, carboplatin, oxaliplatin, gefitinib, erlotinib, TLK-286, cetuximab, bevacizumab, Etoposide, Bleomycin, 5-FU, Melphalan, ZD 6474, ZD 2171, UFT, S1, Ifosfamide, thiotepa, temozolomide, talabostat, Interferon, and combinations thereof; and optionally (b) administering to the patient a maintenance regimen comprising a maintenance dose of a CpG ODN. In a preferred embodiment, the CpG ODN is selected from the group consisting of PF3512676, 1018 ISS, and IMOxine®, and most preferably the CpG ODN is PF3512676.

Preferably, the invention provides a method of treating NSCLC wherein the chemotherapeutic agent is selected from the group consisting of 5-(5-fluoro-2-oxo-1,2-dihydroindol-(3Z)-ylidenemethyl)-2,4-dimethyl-1H-pyrrole-3-carboxylic acid (2-diethylaminoethyl)-amide, Pemetrexed, Gemcitabine, Bevacizumab™, carboplatin, Erlotinib, and combinations thereof, and said therapeutic regimen further comprises administering cisplatin. The invention also specifically contemplates a method of treatment that involves chemotherapy and immunotherapy using a CpG ODN as described above, wherein the method of treatment also includes one or more of surgery, radiation therapy, or a combination thereof.

Still further, the invention provides a method of treating or preventing breast cancer in a patient in need of such treatment, wherein the method comprises (a) a therapeutic regimen comprising administering to the patient simultaneously, semi-simultaneously, separately or sequentially a therapeutically effective amount of a CpG ODN in combination with a therapeutically effective amount of a chemotherapeutic agent selected from the group consisting of 5-(5-fluoro-2-oxo-1,2-dihydroindol-(3Z)-ylidenemethyl)-2,4-dimethyl-1H-pyrrole-3-carboxylic acid (2-diethylaminoethyl)-amide, epirubicin, doxorubicin, paclitaxel, docetaxel, capecitabine, vincristine, Gemcitabine, 5-FU, cyclophosphamide, methotrexate, leucovorin, trastuzumab, bevacizumab, lapatinib, erlotinib, Gefitinib, pemetrexed, tamoxifen, raloxifene, exemestane, anastrozole, zoladex, letrozole, megace, abraxane, bisphosphonate, temozolomide, fragmin, faslodex, irinotecan, cisplatin, carboplatin, oxaliplatin, and combinations thereof; and optionally (b) administering to the patient a maintenance regimen comprising a maintenance dose of a CpG ODN, Preferably, the CpG ODN is selected from the group consisting of PF3512676, 1018 ISS, and IMOxine®, and most preferably the CpG ODN is PF3512676. The invention also specifically contemplates a method of treatment that involves chemotherapy and immunotherapy using a CpG ODN as described above, wherein the method of treatment also includes one or more of surgery, radiation therapy, or a combination thereof.

Moreover, the invention provides a method of treating or preventing melanoma in a patient in need of such treatment, wherein the method comprises (a) a therapeutic regimen comprising administering to the patient simultaneously, semi-simultaneously, separately or sequentially a therapeutically effective amount of a CpG ODN in combination with a therapeutically effective amount of a chemotherapeutic agent selected from the group consisting of DTIC, binblastine, bincristine, vindesine, temozolomide, interferon, interleukin, and combinations thereof; and optionally (b) administering to the patient a maintenance regimen comprising a maintenance dose of a CpG ODN. Preferably, the CpG ODN is selected from the group consisting of PF3512676, 1018 ISS, and IMOxine®, and most preferably, the CpG ODN is PF3512676. The invention also specifically contemplates a method of treatment that involves chemotherapy and immunotherapy using a CpG ODN as described above, wherein the method of treatment also includes one or more of surgery, radiation therapy, or a combination thereof.

Finally, the invention provides a method of treating or preventing renal cell carcinoma in a patient in need of such treatment, wherein the method comprises (a) a therapeutic regimen comprising administering to the patient simultaneously, semi-simultaneously, separately or sequentially a therapeutically effective amount of a CpG ODN in combination with a therapeutically effective amount of a chemotherapeutic agent selected from the group consisting of 5-(5-fluoro-2-oxo-1,2-dihydroindol-(3Z)-ylidenemethyl)-2,4-dimethyl-1H-pyrrole-3-carboxylic acid (2-diethylaminoethyl)-amide, sorafenib, IL-2, Imatinib, Bevacizumab, Gemcitabine, cisplatin, carboplatin, paclitaxel, docetaxel, and combinations thereof; and optionally (b) administering to the patient a maintenance regimen comprising a maintenance dose of a CpG ON. Preferably, the CpG ODN is selected from the group consisting of PF3512676, 1018 ISS, and IMOxine®, and most preferably the CpG ODN is PF3512676. The invention also specifically contemplates a method of treatment that involves chemotherapy and immunotherapy using a CpG ODN as described above, wherein the method of treatment also includes one or more of surgery, radiation therapy, or a combination thereof.

The cancers to be treated may be refractory cancers. A refractory cancer as used herein is a cancer that is resistant to the ordinary standard of care prescribed. These cancers may appear initially responsive to a treatment (and then recur), or they may be completely non-responsive to the treatment. The ordinary standard of care will vary depending upon the cancer type, and the degree of progression in the subject. It may be a chemotherapy, an immunotherapy, surgery, or radiation, or a combination thereof. Those of ordinary skill in the art are aware of such standards of care. Subjects being treated according to the invention for a refractory cancer therefore may have already been exposed to another treatment for their cancer. Alternatively, if the cancer is likely to be refractory (e.g., given an analysis of the cancer cells or history of the subject), then the subject may not have already been exposed to another treatment.

Examples of refractory cancers include but are not limited to leukemias, melanomas, renal cell carcinomas, colon cancer, liver (hepatic) cancers, pancreatic cancer, Non-Hodgkin's lymphoma, and lung cancer.

Therapy Type

The skilled artisan would appreciate, once provided the teachings disclosed herein, that the invention encompasses CpG ODN therapy combined with an chemotherapeutic agent with, or sequentially (preceding or following) with surgery, radiotherapy, or both, to treat cancer. That is, various treatments can be combined with chemotherapeutic agent-CpG ON combination therapy, as would be understood by one skilled in the art once armed with the teachings provided herein.

The methods of the invention in certain instances may be useful for replacing existing surgical procedures or drug therapies, although in other instances the present invention is useful in improving the efficacy of existing therapies for treating such conditions. Accordingly combination therapy may be used to treat the subjects that are undergoing or that will undergo a treatment for inter alia cancer. For example, the agents may be administered to a subject in combination with another anti-proliferative (e.g., an anti-cancer) therapy. Suitable anti-cancer therapies include surgical procedures to remove the tumor mass, chemotherapy or localized radiation. The other anti-proliferative therapy may be administered before, concurrent with, or after treatment with the GpG ODN/chemotherapeutic agent combination of the invention. There may also be a delay of several hours, days and in some instances weeks between the administration of the different treatments, such that the CpG ODN/chemotherapeutic agent combination may be administered before or after the other treatment. The invention further contemplates the use of the CpG ODN/chemotherapeutic agent combination in cancer subjects prior to and following surgery, radiation or chemotherapy.

Thus the invention encompasses use of an chemotherapeutic agent in combination with CpG ODN as a neoadjuvant, adjuvant, first line treatment, second-line and/or third-line therapy, in remission induction or maintenance therapy for cancer. That is, in one embodiment, the chemotherapeutic agent-CpG ODN combination can be co-administered as neoadjuvant therapy prior to, for instance, surgical resection of a tumor (e.g., prostate, breast and lung cancer). In another embodiment, the chemotherapeutic agent-CpG ODN combination can be administered both as a neoadjuvant therapy (i.e., prior to surgery) and also following surgery as an adjuvant therapy. The combination can be used as a first-line treatment instead of another agent (e.g., interferon-alpha).

The methods and compositions of the invention are useful not only in untreated patients but are also useful in the treatment of patients partially or completely unresponsive to other anti-cancer therapies such as but not limited to CpG ODN administered alone or chemotherapeutic agent administered alone. In various embodiments, the invention provides methods and compositions useful for the treatment of diseases or disorders in patients that have been shown to be or may be refractory or non-responsive to therapies comprising the administration of either or both chemotherapeutic agent and/or CpG ODN, and wherein treatment is improved by an enhanced immune response. In one embodiment, the method comprises combining an CpG ODN and an chemotherapeutic agent.

CpG ODN may be used together with an chemotherapeutic agent (as described above) for remission induction, followed by CpG ODN alone for maintenance therapy. Thus, remission induction therapy may require one or more repeated cycles of combination CpG ODN/chemotherapeutic agent therapy. However, once a remission is observed (as is apparent to a medical practitioner), the subject may be placed on maintenance therapy. Such maintenance therapy may involve monotherapy with CpG ODN. For the purpose of maintenance therapy, CpG ODN may be administered once or twice weekly or biweekly, preferably subcutaneously.

While the present invention is exemplified by methods relating to adjuvant, first-line, second-line and/or third-line therapy comprising administering a combination comprising co-administration of an CpG ODN and an chemotherapeutic agent, the skilled artisan, armed with the teachings provided herein, would understand that the invention is not limited to any particular therapy. Rather, methods comprising combined CpG ODN and chemotherapeutic agent therapy encompass use of the combination along the entire disease and treatment continuum.

Thus, the present invention is not limited to use of the combinations of the invention solely for neoadjuvant therapy; instead, the invention includes the entire treatment spectrum, including, but not limited to, adjuvant, first-line, second-line and/or third-line therapy for cancer. This is because the data disclosed herein suggest that immunotherapy comprising an chemotherapeutic agent can provide a therapeutic benefit either alone or combined with at least one additional agent, at any point during treatment. That is, the efficacy of a method that mediates release of tumor-specific antigens, such as cytotoxic therapies (e.g., radiation, chemotherapeutics, and the like), where such antigens are exposed to the immune system, can be enhanced by administration of an chemotherapeutic agent of the invention. Indeed, the data disclosed herein further suggest that a synergistic effect is mediated by combined administration of the chemotherapeutic agent with CpG ODN for treatment of cancer, more particularly, prostate, breast, CRC, melanoma, pancreatic, lung, NSCLC, NHL, RCC, among many cancers. Therefore, the present invention provides important novel therapeutics for treatment of cancer whereby the patient's immune system is enhanced to provide an anti-tumor effect.

In another embodiment, CpG ODN and an chemotherapeutic agent combination is co-administered to enhance and/or prolong an immune response to a tumor. This is because there may be an interaction between the anti-tumor effect of CpG ODN as, inter alia, a TLR9 agonist and the chemotherapeutic agent-mediated blockade of cell signaling of the invention that leads to more effective anti-tumor effect than either agent alone. Therefore, the combination of CpG ODN with an chemotherapeutic agent can provide a potential additive or synergistic effect thereby providing an important novel therapeutic treatment for cancer.

In one embodiment, the invention provides a compositions and methods of producing or increasing an anti-tumor response using an chemotherapeutic agent-CpG ODN combination, wherein CpG ODN enhances an anti-tumor response by an amount of chemotherapeutic agent which is otherwise sub-optimal for inducing the same level of anti-tumor response when used alone. In certain embodiments, when the CpG ODN is not used in conjunction with an chemotherapeutic agent to elicit an anti-tumor response, administering CpG ODN alone does not produce or increase the anti-tumor response. In alternate embodiments, both the CpG ODN and the chemotherapeutic agent can elicit an antitumor response alone and/or when administered in combination.

In certain embodiments, the CpG ODN may enhance the effects of the chemotherapeutic agent (or vice-versa) in an additive manner. In a preferred embodiment, the CpG ODN enhances the effects of the chemotherapeutic agent (or vice versa) in a synergistic manner. In another embodiment, the chemotherapeutic agent enhances the effect of an CpG ODN in an additive manner. Preferably, the effects are enhanced in a synergistic manner. Thus, in certain embodiments, the invention encompasses methods of disease treatment or prevention that provide better therapeutic profiles than administration of CpG ODN alone and chemotherapeutic agent alone.

Encompassed by the invention are combination therapies that have additive potency or an additive therapeutic effect while reducing or avoiding unwanted or adverse effects. The invention also encompasses synergistic combinations where the therapeutic efficacy is greater than additive, while unwanted or adverse effects are reduced or avoided. In certain embodiments, the methods of the invention permit treatment or prevention of diseases and disorders wherein treatment is improved by an enhanced anti-tumor response using lower and/or less frequent doses of chemotherapeutic agent and/or CpG ODN to reduce the incidence of unwanted or adverse effects caused by the administration of chemotherapeutic agent and/or CpG ODN alone, while maintaining or enhancing efficacy of treatment, preferably increasing patient compliance, improving therapy and/or reducing unwanted or adverse effects.

Dosage Regimens

Dosage regimens can be adjusted to provide the optimum desired response. For example, a single bolus can be administered, several divided doses can be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the mammalian subjects to be treated; each unit containing a predetermined quantity of active compound calculated to produce the desired therapeutic effect in association with the required pharmaceutical carrier. The specification for the dosage unit forms of the invention are dictated by and directly dependent on (a) the unique characteristics of the chemotherapeutic agent and the particular therapeutic or prophylactic effect to be achieved, and (b) the limitations inherent in the art of compounding such an active compound for the treatment of sensitivity in individuals.

Thus, the skilled artisan would appreciate, based upon the disclosure provided herein, that the dose and dosing regimen is adjusted in accordance with methods well-known in the therapeutic arts. That is, the maximum tolerable dose can be readily established, and the effective amount providing a detectable therapeutic benefit to a patient can also be determined, as can the temporal requirements for administering each agent to provide a detectable therapeutic benefit to the patient. Accordingly, while certain dose and administration regimens are exemplified herein, these examples in no way limit the dose and administration regimen that can be provided to a patient in practicing the present invention. Further, one skilled in the art would understand, once armed with the teachings provided herein, that a therapeutic benefit, such as, but not limited to, detectable decrease in tumor size and/or metastasis, and increased time to recurrence, among many other parameters, can be assessed by a wide variety of methods known in the art for assessing the efficacy of treatment of cancer, and these methods are encompassed herein, as well as methods to be developed in the future.

It is to be noted that dosage values may vary with the type and severity of the condition to be alleviated, and may include single or multiple doses. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition. For example, doses may be adjusted based on pharmacokinetic or pharmacodynamic parameters, which may include clinical effects such as toxic effects and/or laboratory values. Thus, the present invention encompasses intra-patient dose-escalation as determined by the skilled artisan. Determining appropriate dosages and regiments for administration of the chemotherapeutic agent are well-known in the relevant art and would be understood to be encompassed by the skilled artisan once provided the teachings disclosed herein.

CpG ODN may be administered in one of two dosage types referred to herein as a therapeutic dose and a maintenance dose. Therapeutic dose refers to the dose of CpG ODN administered to a patient during a cycle of therapy in which one or more additional chemotherapeutic agents are administered during the cycle of therapy. For example, if a patient begins 6 cycles of chemotherapy (referred to as a “treatment regimen”), in which one cycle comprises several weekly administrations of CpG ODN, chemotherapeutic agents, or combinations thereof, the doses of GpG ODN administered to the patient in the course of this therapeutic regimen is referred to as a therapeutic dose. In contrast, maintenance dose refers to the dose of CpG ODN administered to a patient after the completion of a therapeutic regimen. The maintenance dose is administered during a “maintenance phase” which is separate in time and distinct in objectives from the treatment regimen.

The therapeutic dose and the maintenance dose may be the same or different at any point in the therapeutic continuum. For example, the patient's initial maintenance dose may be the same as the therapeutic dose, but after a certain interval of time and depending on a variety of factors, including but not limited to stage of disease before and after the completion of a therapeutic regimen, overall well-being of the patient after the completion of a therapeutic regimen, concomitant disease conditions, etc., the maintenance dose may be increased or decreased relative to the therapeutic dose.

CpG ODN can be administered according to standard dosing regimens well known in the art. Subject therapeutic or maintenance doses of CpG ODN for mucosal or local delivery typically range from about 1 μg to 100 mg per administration, which depending on the application could be given daily, weekly, or monthly and any other amount of time therebetween. More typically mucosal or local therapeutic or maintenance doses range from about 100 μg to 50 mg per administration, and most typically from about 1 to 10 mg, with 2-4 administrations being spaced days or weeks apart.

Therapeutic and maintenance doses of the compounds described herein for parenteral delivery for the purpose of inducing a systemic immune response may be typically 2 to 1,000 times higher than the effective mucosal dose, and more typically 2 to 100 times higher, and most typically 5 to 50 times higher.

In a preferred embodiment, therapeutic and maintenance doses of CpG ODN for parenteral (including subcutaneous) delivery for inducing an immune response when CpG ODN is administered in combination with other therapeutic agents, such as the chemotherapeutic agents of the invention, or in specialized delivery vehicles typically range from about 10 μg to 1000 mg per administration, which depending on the application could be given daily, weekly, or monthly and any other amount of time therebetween. More typically parenteral doses for these purposes range from about 1 to 500 mg per administration, and most typically from about 5 to 100 mg, and preferably from about 5 to 50 mg, in 2-4 administrations, spaced days or weeks apart. In some embodiments, however, parenteral doses for these purposes may be used in a range of 5 to 10,000 times higher than the typical doses described above.

In some embodiments, the CpG ODN is administered once weekly in amounts ranging from 10-40 mg total, as either a therapeutic or maintenance dose. In general, CpG ODN may be administered in doses of 5 or 10 mg each, thereby resulting in multiple boll or injections depending on the total amount to be administered. For example, if the total amount to be administered is 10 mg, this may be administered by for example 2×5 mg injection doses, at the same or different injection sites. As another example, if the total amount to be administered is 40 mg, this may be administered by for example 4×10 mg injection doses.

In addition, it will be appreciated that the maintenance dose may administered alone or in combination with one or more additional agents, including but not limited to chemotherapeutic agents and/or palliative agents, depending on a number of factors, including but not limited to the health of the patient, stage of disease, and concomitant disease states. During the maintenance phase, the maintenance dose of CpG ODN may be administered substantially simultaneously or sequentially with one or more additional agents. For substantially simultaneous or sequential administration, the CpG ODN and the one or more additional agents may be in the same or separate formulations or kits.

CpG ODN may be administered substantially simultaneously or sequentially with chemotherapeutic agent(s) of the invention during the therapeutic regimen or maintenance phase. Regardless of the stage of the treatment continuum, when administration is simultaneous, the ODN and the chemotherapeutic agent may be in the same or separate formulations although they are administered at the same time. The term “substantially simultaneously” means that the compounds are administered within minutes of each other (e.g., within 10 minutes of each other) and intends to embrace joint administration as well as consecutive administration, but if the administration is consecutive it is separated in time for only a short period (e.g., the time it would take a medical practitioner to administer two compounds separately). As used herein, concurrent administration and substantially simultaneous administration are used interchangeably. Sequential administration refers to temporally separated administration of the ODN and the chemotherapeutic agent. The separation in time between the administration of these compounds are deliberately longer than the time it takes to administer two medicaments separately, one after the other, without intended delay. Co-administration thus encompasses any temporal combination of administration of the chemotherapeutic agent and the CpG ODN such that administration of the two mediates a therapeutic benefit to the patient that is detectably greater than administration of either agent in the absence of the other.

The CpG ODN may be administered before, concurrently with, or after (or any combination thereof) administration of the chemotherapeutic agent, and vice versa. The CpG ODN may be administered daily (including one or more administrations per day), every other day, every three days, every four days, every five days, every six days, or every week, every month, every two months, every three months, every four months, every five months, every six months, or every year. The chemotherapeutic agent may be administered daily, every other day, every three days, every four days, every five days, every six days, every week, every two weeks, monthly, or every twenty days, every 25 days, every 28 days, every 30 days, every 40 days, every 50 days, every two months, every 70 days, every 80 days, every three months, every six months or yearly. A single dose or multiples doses of the chemotherapeutic agent may be administered. Alternatively, at least one dose, or at least three, six or 12 doses may be administered. The doses may be administered, for example. The administration of the ODN and chemotherapeutic agent may alternate.

In one embodiment, CpG ODN and the chemotherapeutic agent are co-administered in that CpG ODN is administered at the doses recited herein, preferably parenterally (e.g., by subcutaneous or IV route). In another embodiment, the chemotherapeutic agent is administered first to block the inhibitory effects that would limit the efficacy of the CpG ODN. In this embodiment, the chemotherapeutic agent is given preferably from 1 month to 1 day prior to the CpG ODN. In a preferred embodiment, the chemotherapeutic agent is administered at least 1 week but no more than 3 weeks prior to the CpG ODN, and most preferably the chemotherapeutic agent is administered about 1 week prior to the administration of CpG ODN.

In another embodiment, the CpG ODN is given first, to prime the immune system to have a better immune activation response to the chemotherapeutic agent and any other immunotherapies or other therapy that may be given in conjunction with this (e.g., tumor vaccine or etc.). In this embodiment, the CpG ODN is given preferably from 1 month to 1 day prior to the chemotherapeutic agent. In a preferred embodiment, the CpG ODN is administered at least 1 week but no more than 3 weeks prior to the chemotherapeutic agent, and most preferably the CpG ODN is administered about 1 week prior to the administration of chemotherapeutic agent.

While any suitable resting period can be used between administration of CpG ODN and chemotherapeutic agent, the present invention does not require a waiting period and the chemotherapeutic agent and CpG ODN can be co-administered substantially simultaneously. Thus, in one embodiment, the chemotherapeutic agent is administered as a single injection and CpG ODN is administered about 1-7 days either before or after the chemotherapeutic agent.

The chemotherapeutic agent may be administered with the CpG ODN in a multi-day or multi-week cycle or therapeutic regimen. The multi-day cycle may be a 2, 3, 4, 5, 6, 7, 8, 9, or more day cycle, or a 2, 3, 4 or more week cycle. The chemotherapeutic agent may be administered on the first day of such a cycle, followed by administration of the GpG ODN on the first day of each week of a multiweek cycle. For example, the CpG ODN may be administered on days 1, 7 and 14 of a three week cycle. The three week cycle may be repeated once, two three times or more. The entire treatment may be preceded by administration of either the ODN or the chemotherapeutic agent alone, for example in order to prime the immune system or render the subject more responsive to the subsequent therapy.

Additional cycles of chemotherapeutic agent and CpG ODN can be provided as determined by art-recognized methods. However, the present invention is not limited to these or any particular dosage or administration regimens for administering CpG ODN in combination with an chemotherapeutic agent. Rather, the optimal dose, route and regimen for administration of the chemotherapeutic agent and CpG ODN can be readily determined by one of ordinary skill in the relevant art using well-known methods.

The chemotherapeutic agent-CpG ODN combination can be administered as a neoadjuvant therapy prior to surgery, radiation therapy, or any other treatment, in order to sensitize the tumor cells or to otherwise confer a therapeutic benefit to the patient. Additionally, the combination can be co-administered as neoadjuvant therapy following localized treatment (e.g., surgery, radiation, or both).

Further, the combination can be administered as a second line therapy, such as, but not limited to, once any first line therapy has failed. Alternatively, the combination can be administered concurrently with first line therapy, and or at any point during first line therapy, which can be administered following initial treatment. This is because a combination of an chemotherapeutic agent and CpG ODN can provide a therapeutic benefit once first line therapy has failed, once systemic adjuvant therapy has failed, and the like. Thus, the invention encompasses administration of a chemotherapeutic agent and CpG ODN in combination, with or without additional therapy, including, but not limited to, hormonal (e.g., anti-androgen, aromatase inhibitor, and the like), radiotherapy, and any additional therapeutic agent (chemotherapy, signal inhibition therapy, among others), and the like, as would be appreciated by one skilled in the art based upon the disclosure provided herein.

Pharmaceutical Compositions

The invention also relates to an article of manufacture (e.g., dosage form adapted for i.v. administration) comprising a human chemotherapeutic agent in the amount effective to treat cancer (e.g., at least 1 mg/kg, at least 3 mg/kg, at least 5 mg/kg, at least 10 mg/kg, at least 15 mg/kg, or at least 20 mg/kg) and a therapeutically effective amount of CpG ODN. In certain embodiments, the article of manufacture comprises a container or containers comprising a human chemotherapeutic agent, CpG ODN, and a label and/or instructions for use to treat cancer.

The invention encompasses the preparation and use of pharmaceutical compositions comprising a human chemotherapeutic agent of the invention as an active ingredient in combination with and without CpG ODN. Such a pharmaceutical composition may consist of each active ingredient alone, as a combination of at least one active ingredient (e.g., an effective dose of a chemotherapeutic agent, an effective dose of CpG ODN) in a form suitable for administration to a subject, or the pharmaceutical composition may comprise the active ingredient and one or more pharmaceutically acceptable carriers, one or more additional (active and/or inactive) ingredients, or some combination of these.

CpG ODN may be directly administered to the subject or may be administered in conjunction with a nucleic acid delivery complex. A nucleic acid delivery complex shall mean a nucleic acid molecule associated with (e.g. ionically or covalently bound to; or encapsulated within) a targeting means (e.g. a molecule that results in higher affinity binding to target cell. Examples of nucleic acid delivery complexes include oligonucleotides associated with a sterol (e.g. cholesterol), a lipid (e.g. a cationic lipid, virosome or liposome), or a target cell specific binding agent (e.g. a ligand recognized by target cell specific receptor). Preferred complexes may be sufficiently stable in vivo to prevent significant uncoupling prior to internalization by the target cell. However, the complex can be cleavable under appropriate conditions within the cell so that the nucleic acid is released in a functional form.

Delivery vehicles or delivery devices for delivering antigen and oligonucleotides to surfaces have been described. The CpG ODN and/or the antigen and/or other therapeutics may be administered alone (e.g., in saline or buffer) or using any delivery vehicles known in the art. For instance the following delivery vehicles have been described: Cochleates; Emulsomes, ISCOMs; Liposomes; Live bacterial vectors (e.g., Salmonella, Escherichia coli, Bacillus calmatte-guerin, Shigella, Lactobacillus); Live viral vectors (e.g., Vaccinia, adenovirus, Herpes Simplex); Microspheres; Oligonucleotide vaccines; Polymers; Polymer rings; Proteosomes; Sodium Fluoride; Transgenic plants; Virosomes; Virus-like particles, and cationic lipids, peptides, or other carriers that have a charge interaction with the polyanionic oligonucleotide. Other delivery vehicles are known in the art and some additional examples are provided below in the discussion of vectors.

In one embodiment, the chemotherapeutic agent is administered parenterally (e.g., intravenously) in an aqueous solution while the CpG ODN is administered by subcutaneous injection. Preferred formulations and dosage forms of the CpG ODN are described in U.S. Patent Application Publication No. US2004/0198680, the disclosure of which is incorporated herein by reference in its entirety. However, the skilled artisan would understand, based upon the disclosure provided herein, that the invention is not limited to these, or any other, formulations, doses, routes of administration, and the like. Rather, the invention encompasses any formulation or method of administering an chemotherapeutic agent in combination with a CpG ODN, including, but not limited to, administering each agent separately in a different formulation via a different route of administration (e.g., administering an chemotherapeutic agent i.v., while co-administering an CpG ODN subcutaneously, among many others. Thus, the following discussion describes various formulations for practicing the methods of the invention comprising administration of any chemotherapeutic agent in combination with an CpG ODN, but the invention is not limited to these formulations, but comprises any formulation as can be readily determined by one skilled in the art once armed with the teachings provided herein for use in the methods of the invention.

The antibodies employed in the invention can be incorporated into pharmaceutical compositions suitable for administration to a subject. Typically, the pharmaceutical composition comprises the chemotherapeutic agent and a pharmaceutically acceptable carrier. As used herein, “pharmaceutically acceptable carrier” includes any and all solvents, dispersion media, coatings, antibacterial and antifungal agents, isotonic and absorption delaying agents, and the like that are physiologically compatible. Examples of pharmaceutically acceptable carriers include one or more of water, saline, phosphate buffered saline, dextrose, trehalose, glycerol, ethanol and the like, as well as combinations thereof. In many cases, it is preferable to include isotonic agents, for example, sugars, polyalcohols such as mannitol, sorbitol, or sodium chloride in the composition. Pharmaceutically acceptable substances such as wetting or minor amounts of auxiliary substances such as wetting or emulsifying agents, preservatives or buffers, which enhance the shelf life or effectiveness of the chemotherapeutic agent.

The antibodies may be in a variety of forms. These include, for example, liquid, semi solid and solid dosage forms, such as liquid solutions (e.g., injectable and infusible solutions), dispersions or suspensions, tablets, pills, powders, liposomes and suppositories. The preferred form depends on the intended mode of administration and therapeutic application. Typical preferred compositions are in the form of injectable or infusible solutions, such as compositions similar to those used for passive immunization of humans with other antibodies. The preferred mode of administration is parenteral (e.g., intravenous, subcutaneous, intraperitoneal, intramuscular). In a preferred embodiment, the chemotherapeutic agent is administered by intravenous infusion or injection. In another preferred embodiment, the chemotherapeutic agent is administered by intramuscular or subcutaneous injection.

Therapeutic compositions typically must be sterile and stable under the conditions of manufacture and storage. The composition can be formulated as a solution, microemulsion, dispersion, liposome, or other ordered structure suitable to high drug concentration. Sterile injectable solutions can be prepared by incorporating the chemotherapeutic agent in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze drying that yields a powder of the active ingredient plus any additional desired ingredient from a previously sterile filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition an agent that delays absorption, for example, monostearate salts and gelatin.

The chemotherapeutic agents and/or CpG ODN can be administered by a variety of methods known in the art, including, without limitation, oral, parenteral, mucosal, by—inhalation, topical, buccal, nasal, and rectal. For many therapeutic applications, the preferred route/mode of administration is subcutaneous, intramuscular, intravenous or infusion. Non-needle injection may be employed, if desired. As is appreciated by the skilled artisan, the route and/or mode of administration will vary depending upon the desired results.

Dosage regimens may be adjusted to provide the optimum desired response. For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the mammalian subjects to be treated; each unit containing a predetermined quantity of active compound calculated to produce the desired therapeutic effect in association with the required pharmaceutical carrier. The specification for the dosage unit forms of the invention are dictated by and directly dependent on (a) the unique characteristics of the chemotherapeutic agent and the particular therapeutic or prophylactic effect to be achieved, and (b) the limitations inherent in the art of compounding such an active compound for the treatment of sensitivity in individuals.

It is to be noted that dosage values may vary with the type and severity of the condition to be alleviated, and may include single or multiple doses. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition.

In one embodiment, the chemotherapeutic agent is administered in an intravenous formulation as a sterile aqueous solution containing 5 or 10 mg/ml of chemotherapeutic agent, with sodium acetate, polysorbate 80, and sodium chloride at a pH ranging from about 5 to 6. Preferably, the intravenous formulation is a sterile aqueous solution containing 5 or 10 mg/ml of chemotherapeutic agent, with 20 mM sodium acetate, 0.2 mg/ml polysorbate 80, and 140 mM sodium chloride at pH 5.5.

In one embodiment, part of the dose is administered by an intravenous bolus and the rest by infusion of the chemotherapeutic agent formulation. For example, a 0.01 mg/kg intravenous injection of the chemotherapeutic agent may be given as a bolus, and the rest of a predetermined chemotherapeutic agent dose may be administered by intravenous injection. A predetermined dose of the chemotherapeutic agent may be administered, for example, over a period of an hour and a half to two hours to five hours.

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

A pharmaceutical composition of the invention may be prepared, packaged, or sold in bulk, as a single unit dose, or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

The relative amounts of the active ingredient, the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise between 0.1% and 100% (w/w) active ingredient.

In addition to the active ingredient, a pharmaceutical composition of the invention may further comprise one or more additional pharmaceutically active agents. Particularly contemplated additional agents include anti-emetics, anti-diarrheals, chemotherapeutic agents, cytokines, and the like.

Controlled- or sustained-release formulations of a pharmaceutical composition of the invention may be made using conventional technology.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intraperitoneal, intramuscular, intrasternal injection, and kidney dialytic infusion techniques.

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi-dose containers containing a preservative. Formulations for parenteral administration include, but are not limited to, suspensions, solutions, emulsions in oily or aqueous vehicles, pastes, and implantable sustained-release or biodegradable formulations as discussed below. Such formulations may further comprise one or more additional ingredients including, but not limited to, suspending, stabilizing, or dispersing agents. In one embodiment of a formulation for parenteral administration, the active ingredient is provided in dry (i.e. powder or granular) form for reconstitution with a suitable vehicle (e.g. sterile pyrogen-free water) prior to parenteral administration of the reconstituted composition.

A composition of the present invention can be administered by a variety of methods known in the art. The route and/or mode of administration vary depending upon the desired results. The active compounds can be prepared with carriers that protect the compound against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are described by e.g., Sustained and Controlled Release Drug Delivery Systems, J. R. Robinson, ed., Marcel Dekker, Inc., New York, (1978). Pharmaceutical compositions are preferably manufactured under GMP conditions.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a nontoxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides. Other parentally-administrable formulations which are useful include those which comprise the active ingredient in microcrystalline form, in a liposomal preparation, or as a component of a biodegradable polymer systems. Compositions for sustained release or implantation may comprise pharmaceutically acceptable polymeric or hydrophobic materials such as an emulsion, an ion exchange resin, a sparingly soluble polymer, or a sparingly soluble salt.

The chemotherapeutic agent/CpG ODN active ingredient combination of the invention can be administered to an animal, preferably a human. While the precise dosage administered of each active ingredient will vary depending upon any number of factors, including but not limited to, the type of animal and type of disease state being treated, the age of the animal and the route(s) of administration.

An chemotherapeutic agent-CpG ODN combination of the invention may be co-administered with numerous other compounds (antihormonal therapy agents, cytokines, chemotherapeutic and/or antiviral drugs, among many others). Alternatively, the compound(s) may be administered an hour, a day, a week, a month, or even more, in advance of the chemotherapeutic agent-CpG ODN combination, or any permutation thereof. Further, the compound(s) may be administered an hour, a day, a week, or even more, after administration of radiation, stem cell transplant, or administration of any therapeutic agent (e.g., cytokine, chemotherapeutic compound, and the like), or any permutation thereof. The frequency and administration regimen is readily apparent to the skilled artisan and will depend upon any number of factors such as, but not limited to, the type and severity of the disease being treated, the age and health status of the animal, the identity of the compound or compounds being administered, the route of administration of the various compounds, and the like. Several instructive examples demonstrating methods of co-administering an chemotherapeutic agent-CpG ODN to treat cancer are provided, but the invention is not limited in any way to these examples, which merely serve to illustrate methods encompassed by the invention.

Kits

The invention includes various kits for treatment of cancer, The kits comprise a therapeutically effective amount of a human chemotherapeutic agent of the invention and a therapeutically effective amount of CpG ODN, along with an applicator and instructional materials which describe use of the combination to perform the methods of the invention. Although exemplary kits are described below, the contents of other useful kits is apparent to the skilled artisan in light of the present disclosure. Each of these kits is included within the invention.

The invention includes a kit for treatment of renal cell carcinoma in a patient in need thereof. The kit includes a human chemotherapeutic agent of the invention and CpG ODN. The kit further comprises an applicator, including, but not limited to, a syringe, for administration of the components of the kit to a patient. Further, the kit comprises an instructional material setting forth the pertinent information for the use of the kit to treat breast cancer in the patient.

The invention encompasses a kit comprising any combination of an chemotherapeutic agent and CpG ODN. While such kit is preferred, the invention is not limited to this particular combination. Further, the kit can comprise a wide plethora of additional agents for treatment of cancer. Such agents are set forth previously and include chemotherapeutic compounds, cancer vaccines, TLR agonists other than an CpG ODN, other CpG ODNs, receptor tyrosine kinase inhibitors (such as, but not limited to, SU11248), agents useful in treating abnormal cell growth or cancer, antibodies or other ligands that inhibit tumor growth by binding to IGF-1R, a chemotherapeutic agent (taxane, vinca alkaloid, platinum compound, intercalating antibiotics, among many others), and cytokines, among many others, as well as palliative agents to treat, e.g., any toxicities that arise during treatment such as, but not limited to, an anti-diarrheal, an anti-emetic, and the like.

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

### EXAMPLES

### Example 1

### Paclitaxel®/Carboplatin/PF3512676

A clinical trial will be conducted in order to assess the efficacy and safety of PF-3512676 administered in combination with Paclitaxel®/carboplatin chemotherapy as first-line treatment of patients with locally advanced (Stage IIIB with pleural effusion) or metastatic (Stage IV) NSCLC and to compare it to the efficacy and safety of Paclitaxel®/carboplatin chemotherapy alone. The primary objective of this clinical trial will be to compare overall survival (OS) in patients randomized to Paclitaxel®/carboplatin+PF-3512676 (Investigational Treatment Arm) versus that in patients randomized to Paclitaxel®/carboplatin alone (Control Treatment Arm). The secondary objective of the clinical trial will be to compare additional measures of efficacy, safety and health-related quality of life and disease/treatment-related symptoms in patients randomized to Paclitaxel®/carboplatin+PF-3512676 versus patients randomized to Paclitaxel®/carboplatin alone. The trial will also (a) evaluate the effect of PF-3512676 on the pharmacokinetics of paclitaxel and carboplatin; and (b) evaluate the pharmacokinetics of PF-3512676 when administered in combination with paclitaxel/carboplatin.

The primary endpoint of the trial will be overall survival defined as the time from randomization to the date of death due to any cause. The secondary endpoints will include overall confirmed objective response rate (ORR), defined as the proportion of patients with a confirmed best response characterized as either a complete response (CR) or partial response (PR) (target lesions and tumor response defined according to RECIST guidelines). Confirmed responses are those that persist on a follow-up imaging assessment ≧4 weeks after the initial objective documentation of response. Other secondary endpoints will include: (a) Duration of response (DR) defined as the time from first documentation of response to the date of progression or death due to progressive disease, whichever occurs first; (b) Time to tumor progression (TTP) defined as the time from randomization to the date of progression or death due to progressive disease, whichever occurs first; (c) Overall safety profile characterized by type, frequency, severity (as graded using NCI (National Cancer Institute) Common Terminology Criteria for Adverse Events (CTCAE), v3.0 and relationship to study therapy of adverse events and laboratory abnormalities; (d) Patient Reported Outcome (PRO) changes in health-related quality of life and disease/treatment-related symptoms according to EORTC QLQ-C30 and QLQ-LC-13; (e) Pharmacokinetic parameters will include but will not necessarily be limited to Cₘₐₓ and AUC for paclitaxel, carboplatin, and PF-3512676.

Patients enrolled in the trial will be randomized (1:1) to either the investigational treatment arm (Arm A) or to the control treatment arm (Arm B). Patients randomized to Arm A will receive. Standard platinum-based doublet chemotherapy consisting of paclitaxel and carboplatin plus PF-3512676 administered in 3-week cycles. Chemotherapy treatment will be continued for a maximum of 6 cycles. After completion or discontinuation of chemotherapy, for reasons other than disease progression, patients will continue to receive weekly single agent PF-3512676 maintenance. Patients randomized to Arm B will receive: Standard platinum-based doublet chemotherapy consisting of paclitaxel and carboplatin administered in 3-week cycles. Chemotherapy treatment will be continued for a maximum of 6 cycles. Chemotherapy and/or PF-3512676 will be discontinued upon disease progression, unacceptable treatment-related toxicity or patient refusal to continue study treatment. Patients on each arm receive up to 6 cycles of chemotherapy. For each treatment cycle, doses of study treatment are based on patient's actual weight (used in conjunction with baseline height to calculate Body Surface Area) and renal function (using unmodified Cockcroft & Gault formula to estimate GFR from serum creatinine).

Arm A (Investigational Treatment Arm): Paclitaxel+Carboplatin+PF-3512676

Study treatment is administered in 3-week cycles. On Day 1 of each cycle, patients receive: Paclitaxel—200 mg/m², intravenously over 3 hours, Day 1; Carboplatin—AUC 6, intravenously over 15-30 minutes, Day 1 following completion of the paclitaxel infusion. For each treatment cycle, the dose of carboplatin is calculated based on the Calvert formula with a target AUC of 6 mg/ml×min.

Premedication: (a) Hypersensitivity prophylaxis: to minimize the risk of cremophor-related hypersensitivity, standard corticosteroid-based premedication will be administered prior to each dose of paclitaxel, according to the following recommended pretreatment regimen. Dexamethasone 20 mg will be administered orally approximately 12 and 6 hours prior to paclitaxel dosing; if the oral medication is not taken, patients may be treated with IV dexamethasone, at a dose of 20 mg<1 hour prior to paclitaxel dosing; Diphenhydramine 50 mg (or equivalent antihistamine) administered intravenously 30-60 minutes prior to paclitaxel dosing; Cimetidine 300 mg or ranitidine 50 mg (or equivalent H2 blocker) administered intravenously 30-60 minutes prior to paclitaxel dosing; (b) Antiemetic prophylaxis: It is recommended that prophylactic treatment with a 5-HT3 antagonist (eg, granisetron, ondansetron, palonosetron) be administered prior to and for up to 4 days after chemotherapy administration or according to the needs of the patient. The use of concomitant metoclopramide or aprepitant may be considered as alternative following chemotherapy. If the use of corticosteroids for nausea and vomiting is required, it is administered according to the local institutional practices but it is recommended to limit steroid use to 3 days after administration of chemotherapy.

On Day 8 and Day 15 of each cycle, patients will receive: PF-3512676-0.20 mg/kg, subcutaneously. The dose of PF-3512676 will be based on patient's actual weight at the start of each treatment cycle. The site of injection will be on the arm, thigh, hip, back, or the abdominal wall, but upper body sites are preferred. Injection sites should be changed at each administration to limit the impact of local injection reactions. If the total dose exceeds 1.0 mL (15 mg), the injection is divided between 2 sites (eg, half the dose injected in each arm). The maximum dose will be 30 mg (2.0 mL) divided over 2 injection sites. Injection needle size 27 gauge or thinner is recommended for PF-3512676 subcutaneous injection. Injection needle length should be adjusted according to patient's body mass and site of injection to avoid accidental intradermal or intramuscular injection. Premedication: If flu-like symptoms occur after administration of PF-3512676, prophylactic administration of acetaminophen/paracetamol will be considered prior to and following subsequent doses of PF-3512676 to minimize PF-3512676-related flu-like symptoms.

Single Agent PF-3512676 Maintenance: Patients who complete 6 cycles of chemotherapy or who discontinue chemotherapy early for reasons other than disease progression continue to receive weekly single agent PF-351267 maintenance. Single agent PF-3512676 maintenance will be administered in 4-week cycles and continued until disease progression, unacceptable PF-3512676-related toxicity or patient refusal to continue study treatment. The dose of PF-3512676 will be based on patient's actual weight at the start of each treatment cycle.

Arm B (Control Treatment Arm): Paclitaxel+Carboplatin

Study treatment will be administered in 3-week cycles. On Day 1 of each cycle, patients will receive: Paclitaxel—200 mg/m², intravenously over 3 hours, Day 1 Carboplatin—AUC 6, intravenously over 15-30 minutes, Day 1 following completion of the paclitaxel infusion. For each treatment cycle, the dose of carboplatin will be calculated based on the Calvert formula with a target AUC of 6 mg/ml×min. Standard corticosteroid-based premedication is administered prior to each dose of paclitaxel as described above for Arm A.

### Example 2

### Gemcitabine®/Cisplatin/PF3512676

A clinical trial will be run in order to assess the efficacy and safety of PF-3512676 administered in combination with Gemcitabine®/cisplatin chemotherapy as first-line treatment of patients with locally advanced (Stage 111B with pleural effusion) or metastatic (Stage 1V) NSCLC and to compare it to efficacy and safety of Gemcitabine®/cisplatin chemotherapy alone. The primary objective of this clinical trial will be to compare overall survival (OS) in patients randomized to Gemcitabine®/cisplatin+PF-3512676 (Investigational Treatment Arm)) versus that in patients randomized to Gemcitabine®/cisplatin alone (Control Treatment Arm). The secondary objectives for the trials will be to compare additional measures of efficacy, safety and health-related quality of life and disease/treatment-related symptoms in patients randomized to Gemcitabine®/cisplatin+PF-3512676 versus patients randomized to Gemcitabine®/cisplatin alone. Other secondary objectives include: (a) to evaluate the effect of PF-3512676 on the pharmacokinetics of Gemcitabine® and cisplatin; (b) to evaluate the pharmacokinetics of PF-3512676 when administered in combination with Gemcitabine® and cisplatin.

The primary endpoint for this trial will be overall survival, defined as the time from randomization to the date of death due to any cause. The secondary endpoints includes (a) Overall confirmed objective response rate (ORR), defined as the proportion of patients with a confirmed best response characterized as either a complete response (CR) or partial response (PR) (target lesions and tumor response defined according to RECIST guidelines). Confirmed responses are those that persist on a follow-up imaging assessment ≧4 weeks after the initial objective documentation of response. (b) Duration of response (DR), defined as the time from first documentation of response to the date of progression or death due to progressive disease, whichever occurs first. (c) Time to tumor progression (TTP), defined as the time from randomization to the date of progression or death due to progressive disease, whichever occurs first. (d) Overall safety profile characterized by type, frequency, severity [as graded using NCl (National Cancer Institute) Common Terminology Criteria for Adverse Events (CTCAE), v3.0] and relationship to study therapy of adverse events and laboratory abnormalities. (e) Patient Reported Outcome (PRO) changes in health-related quality of life and disease/treatment-related symptoms according to EORTC QLQ-C30 and QLQ-LC-13. (f) Pharmacokinetic parameters to include but not necessarily limited to Cmax and AUC for Gemcitabine®, cisplatin, and PF-3512676.

Patients enrolled in the trial will be randomized (1:1) to either the investigational treatment arm (Arm A) or to the control treatment arm (Arm B). Patients randomized to Arm A will receive standard platinum-based doublet chemotherapy consisting of Gemcitabine® and cisplatin plus PF-3512676 administered in 3-week cycles. Chemotherapy treatment will be continued for a maximum of 6 cycles. After completion or discontinuation of chemotherapy, for reasons other than disease progression, patients will continue to receive weekly single agent PF-3512676 maintenance. Patients randomized to Arm B will receive standard platinum-based doublet chemotherapy consisting of Gemcitabine® and cisplatin administered in 3-week cycles. Chemotherapy treatment is continued for a maximum of 6 cycles. Chemotherapy and/or PF-3512676 will be discontinued upon disease progression, unacceptable treatment-related toxicity, physician decision or patient refusal to continue study treatment.

Arm A (Investigational Treatment Arm): Gemcitabine®+Cisplatin+PF-3512676

Study treatment will be administered in 3-week cycles, On Day 1 of each cycle, premedications will be administered as needed, and study treatment comprising Gemcitabine®, 1250 mg/m2, intravenously over approximately 30 minutes, Cisplatin: 75 mg/m2 intravenously over 1-2 hours, following completion of the Gemcitabine® infusion. On Day 8 of each cycle, premedications will be administered as needed, followed by study treatment, comprising Gemcitabine®: 1250 mg/m2, intravenously over approximately 30 minutes, followed by PF-3512676: 0.20 mg/kg, subcutaneously. The site of injection may be on the arm, thigh, hip, back, or the abdominal wall, but upper body sites are preferred. Injection sites should be changed at each administration to limit the impact of local injection reactions. If the total dose exceeds 1.0 mL (15 mg), the injection is divided between 2 sites (e.g., half the dose injected in each arm). The maximum dose is 30 mg (2.0 mL) divided over 2 injection sites. Injection needle size 27 gauge or thinner is recommended for PF-3512676 subcutaneous injection. Injection needle length should be adjusted according to patients body mass and site of injection to avoid accidental intradermal or intramuscular injection. If flu-like symptoms occur after administration of PF-3512676, prophylactic administration of acetaminophen/paracetamol should be considered prior to and following subsequent doses of PF-3512676 to minimize PF-3512676-related flu-like symptoms. Patients will be observed at the clinical study site for at least 1-hour after PF-3512676 administration. On Day 15 of each cycle, premedications will be administered as needed, followed by study treatment comprising PF-3512676: 0.20 mg/kg, subcutaneously.

Single Agent PF-3512676 Maintenance

Patients who complete chemotherapy or who discontinue chemotherapy early for reasons other than disease progression will continue to receive weekly single agent PF-3512676 maintenance; the first cycle beginning 3 weeks after Day 1 of the final cycle of chemotherapy. Single agent PF-3512676 maintenance will be administered in 4-week cycles and will be continued until disease progression, unacceptable PF-3512676-related toxicity or patient refusal to continue study treatment. The dose of PF-3512676 will be based on patients actual weight at the start of each treatment cycle.

Arm B (Control Treatment Arm): Gemcitabine®+Cisplatin

Study treatment is administered in 3-week cycles. On Day 1 of each cycle, premedications as needed, followed by study treatment comprising Gemcitabine®: 1250 mg/m2, intravenously over approximately 30 min., cisplatin: 75 mg/m2 intravenously over 1-2 hours, following completion of the Gemcitabine® infusion. On Day 8 of each cycle, premedications will be administered as needed, followed by study treatment comprising Gemcitabine®: 1250 mg/m2, intravenously over approximately 30 minutes. Premedications include but are not limited to antiemetic prophylaxis of cisplatin-related emesis will be treated by the following treatment schedules: Day 1 (Acute emesis): a 5HT3 antagonist (see below list of 5HT3 antagonists), plus dexamethasone 20 mg (oral or IV over 5 to 15 minutes) will be given once within 1 hour of chemotherapy.

Dolasetron 100 mg oral or IV

Granisetron 1 or 2 mg oral, or 0.1 mg/kg or 1 mg IV

Ondansetron 24 mg oral or 0.15 mg/kg IV or 8 mg IV

Aprepitant 125 mg oral may also be used

On day 2-4 (Delayed emesis): delayed emesis medications should be commenced the morning of Day 2 and continued twice daily (bid) for 3 days. Dexamethasone 8 mg oral bid plus metoclopramide 30 to 40 mg oral bid for 3 days is recommended. A 5HT3 antagonist or Aprepitant 80 mg oral may be substituted for metoclopramide. On day 8: Antiemetic prophylaxis will be at investigator discretion and may be carried out according to standard local practice. If flu-like symptoms occur after administration of PF-3512676, prophylactic administration of acetaminophen/paracetamol should be considered prior to and following subsequent doses of PF-3512676 to minimize PF-3512676-related flu-like symptoms. Pre and post cisplatin hydration is mandatory to minimize cisplatin renal toxicity. Hydration should be performed according to standard local practice or the following schedule: Pre cisplatin hydration: ≧2 L of any fluid taken orally on the day prior to chemotherapy and ≧1 L orally on the morning of chemotherapy plus a minimum of 500 mL of intravenous fluid given on the morning of cisplatin treatment (the 500 mL of IV fluid may be part of the Gemcitabine® infusion). Post cisplatin hydration: immediately after cisplatin, an IV infusion of 200-250 mL/hr of 5% dextrose in ½ normal saline (D51/2NS), 5% dextrose in normal saline (D5NS), or normal saline (NS) and 30 meq KCL/L is given for 4-6 hours.

Examples 3-4 and 5-6 relate to the use of 1018 ISS and IMOxine, respectively, as the CpG ODN in combination studies as described above in Examples 1 and 2. Therefore, the studies are conducted in much the same way as described above in Examples 1 and 2 and as summarized in the following table.

TABLE 9
Examples 3-6: Use of CpG ODNs 1018 ISS and
IMOxine in Combination Therapies

| | CpG | | |
|---|---|---|---|
| Example | ODN | Arm A | Arm B |
| 3 | 1018 ISS | Paclitaxel/Carboplatin/1018 ISS | Paclitaxel/Carboplatin |
| | | Study treatment is administered in | Study treatment will be |
| | | 3-week cycles. On Day 1 of each | administered in 3-week |
| | | cycle, patients receive: Paclitaxel - | cycles. On Day 1 of each |
| | | 200 mg/m², intravenously over 3 | cycle, patients will receive: |
| | | hours, Day 1; Carboplatin - AUC 6, | Paclitaxel - 200 mg/m², |
| | | intravenously over 15-30 minutes, | intravenously over 3 hours, |
| | | Day 1 following completion of the | Day 1 Carboplatin - AUC 6, |
| | | paclitaxel infusion. For each | intravenously over 15-30 |
| | | treatment cycle, the dose of | minutes, Day 1 following |
| | | carboplatin is calculated based on the | completion of the paclitaxel |
| | | Calvert formula with a target AUC of | infusion. For each treatment |
| | | 6 mg/ml × min. Premedication as | cycle, the dose of |
| | | described in Arm A of Example 1 | carboplatin will be |
| | | above. | calculated based on the |
| | | On Day 8 and Day 15 of each cycle, | Calvert formula with a target |
| | | patients will receive: 1018 ISS - 0.20 mg/kg, | AUC of 6 mg/ml × min. |
| | | subcutaneously. The dose of | Standard corticosteroid- |
| | | 1018 ISS will be based on patient's | based premedication is |
| | | actual weight at the start of each | administered prior to each |
| | | treatment cycle. Choice of injection | dose of paclitaxel as |
| | | site and administration at injection site | described above for Arm A. |
| | | as described above in Example 1 | |
| | | above. Premedication for 1018 ISS | |
| | | administration: As described above in | |
| | | Example 1. | |
| | | Single Agent 1018 ISS Maintenance: | |
| | | Patients that complete 6 cycles of | |
| | | chemotherapy or who discontinue | |
| | | chemotherapy early for reasons other | |
| | | than disease progression continue to | |
| | | receive weekly single agent | |
| | | PF-351267 maintenance. Single | |
| | | agent 1018 ISS maintenance will be | |
| | | administered in 4-week cycles and | |
| | | continued until disease progression, | |
| | | unacceptable 1018 ISS-related | |
| | | toxicity or patient refusal to continue | |
| | | study treatment. The dose of 1018 | |
| | | ISS will be based on patient's actual | |
| | | weight at the start of each treatment | |
| | | cycle. | |
| 4 | IMOxine | Gemcitabine ®/Cisplatin/1018 ISS | Gemcitabine ®/cisplatin |
| | | Study treatment will be administered | Study treatment is |
| | | in 3-week cycles. On Day 1 of each | administered in 3-week |
| | | cycle, premedications will be | cycles. On Day 1 of each |
| | | administered as needed, and study | cycle, premedications as |
| | | treatment comprising Gemcitabine ®, | needed, followed by study |
| | | 1250 mg/m2, intravenously over | treatment comprising |
| | | approximately 30 minutes, Cisplatin: | Gemcitabine ®: 1250 mg/m2, |
| | | 75 mg/m2 intravenously over 1-2 | intravenously over |
| | | hours, following completion of the | approximately 30 minutes, |
| | | Gemcitabine ® infusion. On Day 8 of | cisplatin: 75 mg/m2 |
| | | each cycle, premedications will be | Intravenously over 1-2 |
| | | administered as needed, followed by | hours, following completion |
| | | study treatment, comprising | of the Gemcitabine ® |
| | | Gemcitabine ®: 1250 mg/m2, | infusion. On Day 8 of each |
| | | intravenously over approximately 30 | cycle, premedications will |
| | | minutes, followed by 1018 ISS: 0.20 mg/kg, | be administered as needed, |
| | | subcutaneously. The choice | followed by study treatment |
| | | of injection site and manner of | comprising Gemcitabine ®: |
| | | injection may be selected according to | 1250 mg/m2, Intravenously |
| | | the methods outlined in Example 2 | over approximately 30 |
| | | above. On Day 15 of each cycle, | minutes. Premedications |
| | | premedications will be administered | administered as described |
| | | as needed, followed by study | above in Example 2. |
| | | treatment comprising 1018 ISS: 0.20 mg/kg, | |
| | | subcutaneously. | |
| | | Single Agent 1018 ISS Maintenance | |
| | | Patients who complete chemotherapy | |
| | | or who discontinue chemotherapy | |
| | | early for reasons other than disease | |
| | | progression will continue to receive | |
| | | weekly single agent 1018 ISS | |
| | | maintenance; the first cycle beginning | |
| | | 3 weeks after Day 1 of the final cycle | |
| | | of chemotherapy. Single agent 1018 | |
| | | ISS maintenance will be administered | |
| | | in 4-week cycles and will be continued | |
| | | until disease progression, | |
| | | unacceptable 1018 ISS-related | |
| | | toxicity or patient refusal to continue | |
| | | study treatment. The dose of 1018 | |
| | | ISS will be based on patient's actual | |
| | | weight at the start of each treatment | |
| | | cycle. | |
| 5 | IMOxine | Paclitaxel/Carboplatin/IMOxine ® | Paclitaxel/Carboplatin |
| | | Study treatment is administered in | Study treatment will be |
| | | 3-week cycles. On Day 1 of each | administered in 3-week |
| | | cycle, patients receive: Paclitaxel - | cycles. On Day 1 of each |
| | | 200 mg/m², intravenously over 3 | cycle, patients will receive: |
| | | hours, Day 1; Carboplatin - AUC 6, | Paclitaxel - 200 mg/m², |
| | | intravenously over 15-30 minutes, | intravenously over 3 hours, |
| | | Day 1 following completion of the | Day 1 Carboplatin - AUC 6, |
| | | paclitaxel infusion. For each | intravenously over 15-30 |
| | | treatment cycle, the dose of | minutes, Day 1 following |
| | | carboplatin is calculated based on the | completion of the paclitaxel |
| | | Calvert formula with a target AUC of | infusion. For each treatment |
| | | 6 mg/ml × min. Premedication is | cycle, the dose of |
| | | administered as described above in | carboplatin will be |
| | | Example 1. | calculated based on the |
| | | On Day 8 and Day 15 of each cycle, | Calvert formula with a target |
| | | patients will receive: IMOxine ® - 0.20 mg/kg, | AUC of 6 mg/ml × min. |
| | | subcutaneously. The dose of | Premedication administered |
| | | IMOxine ® will be based on patient's | as described above in |
| | | actual weight at the start of each | Example 1. |
| | | treatment cycle. The site of injection | |
| | | and manner of injection is determined | |
| | | as described above in Example 1. | |
| | | Single Agent IMOxine ® Maintenance: | |
| | | Patients who complete 6 cycles of | |
| | | chemotherapy or who discontinue | |
| | | chemotherapy early for reasons other | |
| | | than disease progression continue to | |
| | | receive weekly single agent | |
| | | IMOxine ® maintenance. Single agent | |
| | | IMOxine ® maintenance will be | |
| | | administered in 4-week cycles and | |
| | | continued until disease progression, | |
| | | unacceptable IMOxine ®-related | |
| | | toxicity or patient refusal to continue | |
| | | study treatment. The dose of | |
| | | IMOxine ® will be based on patient's | |
| | | actual weight at the start of each | |
| | | treatment cycle. | |
| 6 | IMOxine | Gemcitabine ®/Cisplatin/IMOxine ® | Gemcitabine ®/cisplatin |
| | | Study treatment will be administered | Study treatment is |
| | | in 3-week cycles. On Day 1 of each | administered in 3-week |
| | | cycle, premedications will be | cycles. On Day 1 of each |
| | | administered as needed, and study | cycle, premedications as |
| | | treatment comprising Gemcitabine ®, | needed, followed by study |
| | | 1250 mg/m2, intravenously over | treatment comprising |
| | | approximately 30 minutes, Cisplatin: | Gemcitabine ®: 1250 mg/m2, |
| | | 75 mg/m2 intravenously over 1-2 | intravenously over |
| | | hours, following completion of the | approximately 30 min., |
| | | Gemcitabine ® infusion. On Day 8 of | cisplatin: 75 mg/m2 |
| | | each cycle, premedications will be | intravenously over 1-2 |
| | | administered as needed, followed by | hours, following completion |
| | | study treatment, comprising | of the Gemcitabine ® |
| | | Gemcitabine ®: 1250 mg/m2, | infusion. On Day 8 of each |
| | | intravenously over approximately 30 | cycle, premedications will |
| | | minutes, followed by IMOxine ®: 0.20 mg/kg, | be administered as needed, |
| | | subcutaneously. The site of | followed by study treatment |
| | | injection and manner of injection may | comprising Gemcitabine ®: |
| | | be determined as described above in | 1250 mg/m2, intravenously |
| | | Example 2. On Day 15 of each cycle, | over approximately 30 |
| | | premedications will be administered | minutes. Premedications |
| | | as needed, followed by study | administered as described |
| | | treatment comprising IMOxine ®: 0.20 mg/kg, | above in Example 2. |
| | | subcutaneously. | |
| | | Single Agent IMOxine ® Maintenance | |
| | | Patients who complete chemotherapy | |
| | | or who discontinue chemotherapy | |
| | | early for reasons other than disease | |
| | | progression will continue to receive | |
| | | weekly single agent IMOxine ® | |
| | | maintenance; the first cycle beginning | |
| | | 3 weeks after Day 1 of the final cycle | |
| | | of chemotherapy. Single agent | |
| | | IMOxine ® maintenance will be | |
| | | administered in 4-week cycles and will | |
| | | be continued until disease | |
| | | progression, unacceptable IMOxine ®- | |
| | | related toxicity or patient refusal to | |
| | | continue study treatment. The dose | |
| | | of IMOxine ® will be based on | |
| | | patient's actual weight at the start of | |
| | | each treatment cycle. | |

### REFERENCES

1. Jemal A., Murray T., Ward E., et al., Cancer Statistics, 2005. CA 55: 10-30, 2005.
2. Non-small Cell Lung Cancer Collaborative Group. BMJ 311: 899-909, 1995.
3. Kelly K., Crowley J., Bunn P., et al., J. Clin. Oncol., 19: 3210-8, 2001.
4. Schiller J. H., Harrington D., Belani C. P., et al., N. Engl, J. Med., 346: 92-98, 2002.
5. Scagliotti G., De Marinis F., Rinaldi M., et al., J. Clin. Oncol., 20: 4285-91, 2002.
6. Smit E. F., van Meerbeeck J. P., Lianes P., et al. J. Clin. Oncol., 21(21): 3909-17, Nov. 1, 2003.
7. Alberola V., Camps C., Provencia M., et al., J. Clin. Oncol., 21: 3207-13, 2003.
8. Delbaldo C., Michiels S., Syz N., et al., JAMA, 292 (4): 470-484, Jul. 28, 2004.
9. Argiris A., Schiller J., JAMA, 292 (4): 499-500, Jul. 28, 2004.
10. Herbst R. S., Prager D., Hermann R., et al., J. Clin. Oncol., 22:14s, 2004.
11. Shepherd F. A., Pereira J., Ciuleanu T. E., et al., J. Clin. Oncol., 22:622s, 2004.
12. Cohen M. H., Williams G. A., Sridhara R., et al., Clin. Cancer Res., 10:1212-1218, 2004.
13. Fukuoka M., Yano S., Giaccone G., et al., J. Clin. Oncol. 21:2237-2246, 2003.
14. Kris M. G., Natale R. B., Herbst R. S., et al., JAMA 290:2149-2158, 2003.
15. Herbst R., Giaccone G., Schiller J., et al., J. Clin. Oncol., Vol 22(5): 785-794, Mar. 1, 2004.
16. Giaccone G., Herbst R., Manegold C., et al., J. Clin. Oncol., Vol 22 (5): 777-784, Mar. 1, 2004.
17. Sandier A. R., Gray R. J., Brahmer J., et al., A. Proc. Am. Soc. Clin. Oncol., 23:LBA4, 2005.
18. Butts, et al., 3INa. ESMO Meeting, 2004. Ann. Oncol., Vol, 15(S3), 2004.
19. Krieg A. M., Yi A. K., Matson S., et al., Nature, 6:374(6522):546-9, April 1995.
20. Krieg A. M., Annu Rev Immunol., 2002; 20:709-60.
21. American Society of Clinical Oncology, JCO 1996; 14 (6); 1957-1960.
22. Reece P A, Stafford I, Abbott R L, et al., J. Clin. Oncol., 7:270-275, 1989.
23. Dy G. K., Suri A., Reid J. M., et al., Cancer Chemother Pharmacol., 55:522-530, 2005.
24, Crockcroft D W, Gault H, Nephron 16:31-41, 1976.
25. Calvert A H, Newell D R, Gumbrell L A, et al. J Clin Oncol 7:1748-1756, 1989.

The present invention is not to be limited in scope by the specific embodiments described herein. Indeed, various modifications of the invention in addition to those described herein will become apparent to those skilled in the art from the foregoing description and the accompanying FIGURE. Such modifications are intended to fall within the scope of the appended claims.

All patents, applications, publications, test methods, literature, and other materials cited herein are hereby incorporated herein by reference in their entireties.

1. A method of treating or preventing NSCLC in a patient in need of such treatment, wherein the method comprises (a) a therapeutic regimen comprising administering to the patient simultaneously, semi-simultaneously, separately or sequentially a therapeutically effective amount of a CpG ODN in combination with a therapeutically effective amount of (a) a first chemotherapeutic agent selected from the group consisting of 5-(5-fluoro-2-oxo-1,2-dihydroindol-(3Z)-ylidenemethyl)-2,4-dimethyl-1H-pyrrole-3-carboxylic acid (2-diethylaminoethyl)-amide, paclitaxel, docetaxel, gemcitabine, vinorelbine, irinotecan, pemetrexed, mitomycin, vincristine, vinblastine, vindesine, cisplatin, carboplatin, oxaliplatin, gefitinib, erlotinib, TLK-286, cetuximab, bevacizumab, etoposide, bleomycin, 5-FU, melphalan, ZD 6474, ZD 2171, UFT, S1, ifosfamide, thiotepa, temozolomide, talabostat, interferon; and (b) a second chemotherapeutic agent selected from the group consisting of 5-(5-fluoro-2-oxo-1,2-dihydroindol-(3Z)-ylidenemethyl)-2,4-dimethyl-1H-pyrrole-3-carboxylic acid (2-diethylaminoethyl)-amide, paclitaxel, docetaxel, gemcitabine, vinorelbine, irinotecan, pemetrexed, mitomycin, vincristine, vinblastine, vindesine, cisplatin, carboplatin, oxaliplatin, gefitinib, erlotinib, TLK-286, cetuximab, bevacizumab, etoposide, bleomycin, 5-FU, melphalan, ZD 6474, ZD 2171, UFT, S1, ifosfamide, thiotepa, temozolomide, talabostat, interferon; wherein said first and second chemotherapeutic agents are different; and optionally (c) administering to the patient a maintenance regimen comprising a maintenance dose of a CpG ODN; with the proviso that if said first chemotherapeutic agent is selected from cisplatin or carboplatin then the second chemotherapeutic agent is not paclitaxel or docetaxel.

2. The method according to claim 1 wherein said CpG ODN is selected from the group consisting of PF3512676, 1018 ISS, Genazense, and IMOxine®.

3. The method according to claim 2 wherein the CpG ODN is PF3512676.

4. The method according to claim 1 wherein the therapeutically effective amount of a CpG ODN is a therapeutic dose of about 0.01 to 5.0 mg/kg.

5. The method according to claim 4 wherein the therapeutically effective amount of a CpG ODN is a therapeutic dose of about 0.01 to 2.5 mg/kg.

6. The method according to claim 5 wherein the therapeutically effective amount of a GpG ODN is a therapeutic dose of about 0.05 to 1.0 mg/kg.

7. The method according to claim 6 wherein the therapeutically effective amount of a CpG ODN is a therapeutic dose of about 0.2 mg/kg.

8. The method according to claim 1 wherein the therapeutic dose is administered before administration of the chemotherapeutic agent.

9. The method according to claim 1 wherein the therapeutic dose is administered after administration of the chemotherapeutic agent.

10. The method according to claim 1 wherein the therapeutic dose is administered to the patient about 1 week to 3 weeks before the administration of the chemotherapeutic agent.

11. The method according to claim 10 wherein the therapeutic dose is administered to the patient about 1 week before the administration of the chemotherapeutic agent.

12. The method according to claim 1 wherein the therapeutic dose is administered to the patient about 1 week to 3 weeks after administration of the chemotherapeutic agent.

13. The method according to claim 12 wherein the therapeutic dose is administered to the patient about 1 week after administration of the chemotherapeutic agent.

14. The method according to claim 1 wherein the method further comprises a treatment regimen comprising a therapy selected from the group consisting of surgery, radiation therapy, or a combination thereof.

15. The method according to claim 1 wherein the maintenance dose of a CpG ODN is about 0.01 to 5.0 mg/kg.

16. The method according to claim 15 wherein the maintenance dose of a CpG ODN is about 0.01 to 2.5 mg/kg.

17. The method according to claim 16 wherein the maintenance dose of a CpG ODN is about 0.05 to 1.0 mg/kg.

18. The method according to claim 17 wherein the maintenance dose of a CpG ODN is about 0.2 mg/kg.
